(19) United States
(12) Patent Application Publication
(10) Pub. No.: US 2014/0296266 A1
(43) Pub. Date: Oct. 2, 2014

(54) THERAPEUTIC COMPOUNDS

(71) Applicant: Gilead Sciences, Inc., Foster City, CA (US)

(72) Inventors: Yunfeng Eric HU, San Mateo, CA (US); John O. LINK, San Francisco, CA (US); Qi LIU, Union City, CA (US); Winston C. TSE, Redwood City, CA (US); Jennifer R. ZHANG, Union City, CA (US)

(21) Appl. No.: 14/194,611

(22) Filed: Feb. 28, 2014

Related U.S. Application Data
(60) Provisional application No. 61/771,655, filed on Mar. 1, 2013.

(51) Int. Cl. C07D 471/04; C07D 401/14; A61K 45/06; A61K 31/4439; C07D 401/12; A61K 31/437; A61K 31/517

(52) U.S. Cl. CPC C07D 471/04; A61K 31/437; C07D 401/14; A61K 31/517; A61K 31/4439; C07D 401/12; A61K 45/06
USPC 514/266.21; 546/113; 514/300; 544/284; 546/275.7; 514/338; 546/256; 514/333

(57) ABSTRACT

Compounds of formula I:

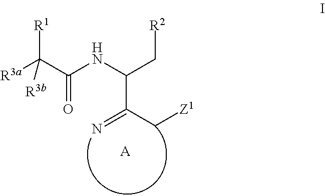

or salts thereof are disclosed. Also disclosed are pharmaceutical compositions comprising a compound of formula I, processes for preparing compounds of formula I, intermediates useful for preparing compounds of formula I and therapeutic methods for treating a Retroviridae viral infection including an infection caused by the HIV virus.

### CROSS-REFERENCE TO RELATED APPLICATIONS

This application claims the benefit of and priority to U.S. Provisional Patent Application Ser. No. 61/771,655, filed Mar. 1, 2013, the disclosure of which is hereby incorporated herein by reference in its entirety.

### BACKGROUND

Positive-single stranded RNA viruses comprising the Retroviridae family include those of the subfamily Orthoretrovirinae and genera Alpharetrovirus, Betaretrovirus, Gamaretrovirus, Deltaretrovirus, Epsilonretrovirus, Lentivirus, and Spumavirus which cause many human and animal diseases. Among the Lentivirus, HIV-1 infection in humans leads to depletion of T helper cells and immune dysfunction, producing immunodeficiency and vulnerability to opportunistic infections. Treating HIV-1 infections with highly active antiretroviral therapies (HAART) has proven to be effective at reducing viral load and significantly delaying disease progression (Hammer, S. M., et al.; JAMA 2008, 300: 555-570). However, these treatments do lead to the emergence of HIV strains that are resistant to current therapies (Taiwo, B., International Journal of Infectious Diseases 2009, 13:552-559; Smith, R. J., et al., Science 2010, 327:697-701). Therefore, there is a pressing need to discover new antiretroviral agents including agents that are active against emerging drug-resistant HIV variants.

### SUMMARY

Provided herein are compounds and methods for the treatment of a viral infection.

One embodiment provides a compound of formula I

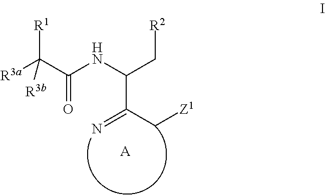

wherein:

A is a 6-membered monocyclic-heteroaryl with one or two nitrogen atoms, wherein the 6-membered monocyclic-heteroaryl is substituted with one Z¹ group at the position shown, one Z² group, and optionally substituted with one or more (e.g., 1 or 2) Z³ groups;

R¹ is 6-12 membered aryl, 5-12 membered heteroaryl or 3-12 membered heterocycle, wherein any 6-12 membered aryl, 5-12 membered heteroaryl or 3-12 membered heterocycle of R¹ is optionally substituted with one or more (e.g., 1, 2, 3, 4 or 5) Z⁴ groups;

R² is phenyl, 5-membered monocyclic-heteroaryl, 6-membered monocyclic-heteroaryl or (C₃-C₇)carbocycle, wherein any phenyl, 5-membered monocyclic-heteroaryl, 6-membered monocyclic-heteroaryl or (C₃-C₇)carbocycle of R² is optionally substituted with one or more (e.g., 1, 2, 3, 4 or 5) Z⁵ groups;

each R^{3a} and R^{3b} is independently selected from H, halogen, (C₁-C₃)alkyl and (C₁-C₃)haloalkyl, or R^{3a} is selected from H, (C₁-C₃)alkyl and (C₁-C₃)haloalkyl and R^{3b} is selected from —OH and —CN;

Z¹ is selected from 6-12 membered aryl, 5-14 membered heteroaryl and 3-14 membered heterocycle, wherein any 6-12 membered aryl, 5-14 membered heteroaryl and 3-14 membered heterocycle of Z¹ is optionally substituted with one or more (e.g., 1, 2, 3, 4 or 5) Z^{1a} or Z^{1b};

each Z^{1a} is independently selected from (C₃-C₇)carbocycle, 6-12 membered aryl, 5-12 membered heteroaryl, 3-12 membered heterocycle, halogen, —CN, —ORⁿ¹, —OC(O)R^{p1}, —OC(O)NR^{q1}R^{r1}, —SRⁿ¹, —S(O)R^{p1}, —S(O)₂OH, —S(O)₂R^{p1}, —S(O)₂NR^{q1}R^{r1}, —NR^{q1}R^{r1}, —NRⁿ¹COR^{p1}, —NRⁿ¹CO₂R^{p1}, —NRⁿ¹CONR^{q1}R^{r1}, —NRⁿ¹S(O)₂R^{p1}, —NRⁿ¹S(O)₂OR^{p1}, —NRⁿ¹S(O)₂NR^{q1}R^{r1}, NO₂, —C(O)Rⁿ¹, —C(O)ORⁿ¹, —C(O)NR^{q1}R^{r1} and —S(O)₂NRⁿ¹COR^{p1}, wherein any (C₃-C₇)carbocycle, 6-12 membered aryl, 5-12 membered heteroaryl and 3-12 membered heterocycle of Z^{1a} is optionally substituted with one or more (e.g., 1, 2, 3, 4 or 5) Z^{1c} or Z^{1d} groups;

each Z^{1b} is independently selected from (C₁-C₈)alkyl, (C₂-C₈)alkenyl and (C₂-C₈)alkynyl, wherein any (C₁-C₈)alkyl, (C₂-C₈)alkenyl and (C₂-C₈)alkynyl of Z^{1b} is optionally substituted with one or more (e.g., 1, 2, 3, 4 or 5) Z^{1c} groups;

each Z^{1c} is independently selected from (C₃-C₇)carbocycle, phenyl, 5-6 membered monocyclic-heteroaryl, 3-7 membered heterocycle, halogen, —CN, —ORⁿ², —OC(O)R^{p2}, —OC(O)NR^{q2}R^{r2}, —SRⁿ², —S(O)R^{p2}, —S(O)₂OH, —S(O)₂R^{p2}, —S(O)₂NR^{q2}R^{r2}, —NR^{q2}R^{r2}, —NRⁿ²COR^{p2}, —NRⁿ²CO₂R^{p2}, —NRⁿ²CONR^{q2}R^{r2}, —NRⁿ²S(O)₂R^{p2}, —NRⁿ²S(O)₂OR^{p2}, —NRⁿ²S(O₂NR^{q2}R^{r2}, NO₂, —C(O)Rⁿ², —C(O)ORⁿ², —C(O)NR^{q2}R^{r2}, halophenyl, 5-6 membered haloheteroaryl, 3-7 membered haloheterocycle and (C₁-C₈)heteroalkyl;

each Z^{1d} is independently selected from (C₁-C₈)alkyl, (C₂-C₈)alkenyl, (C₂-C₈)alkynyl and (C₁-C₈)haloalkyl;

each Rⁿ¹ is independently selected from H, (C₁-C₈)alkyl, (C₂-C₈)alkenyl, (C₂-C₈)alkynyl, (C₃-C₇)carbocycle, 3-7 membered heterocycle, 5-6 membered monocyclic-heteroaryl and phenyl, wherein any (C₃-C₇)carbocycle, 3-7 membered heterocycle, 5-6 membered monocyclic-heteroaryl and phenyl of Rⁿ¹ is optionally substituted with one or more (e.g., 1, 2, 3, 4 or 5) Z^{1c} or Z^{1d} groups, and wherein any (C₁-C₈)alkyl, (C₂-C₈)alkenyl and (C₂-C₈)alkynyl of Rⁿ¹ is optionally substituted with one or more (e.g., 1, 2, 3, 4 or 5) Z^{1c} groups;

each R^{p1} is independently selected from (C₁-C₈)alkyl, (C₂-C₈)alkenyl, (C₂-C₈)alkynyl, (C₃-C₇)carbocycle, 3-7 membered heterocycle, 5-6 membered monocyclic-heteroaryl and phenyl, wherein any (C₃-C₇)carbocycle, 3-7 membered heterocycle, 5-6 membered monocyclic-heteroaryl and phenyl of R^{p1} is optionally substituted with one or more (e.g., 1, 2, 3, 4 or 5) Z^{1c} or Z^{1d} groups, and wherein any (C₁-C₈)alkyl, (C₂-C₈)alkenyl and (C₂-C₈)alkynyl of R^{p1} is optionally substituted with one or more (e.g., 1, 2, 3, 4 or 5) Z^{1c} groups;

R^{q1} and R^{r1} are each independently selected from H, (C₁-C₈)alkyl, (C₂-C₈)alkenyl, (C₂-C₈)alkynyl, (C₃-C₇)carbocycle, 3-7 membered heterocycle, 5-6 membered monocyclic-heteroaryl and phenyl, wherein any (C₃-C₇)carbocycle, 3-7 membered heterocycle, 5-6 membered monocyclic-heteroaryl and phenyl of R^{q1} or R^{r1} is optionally substituted with one or more (e.g., 1, 2, 3, 4 or 5) Z^{1c} or Z^{1d} groups, and wherein any (C₁-C₈)alkyl, (C₂-C₈)alkenyl and (C₂-C₈)alkynyl of R^{q1} or R^{r1} is optionally substituted with one or more (e.g., 1, 2, 3, 4 or 5) Z^{1c} groups, or R^{q1} and R^{r1} together with the nitrogen to which they are attached form a 5, 6 or 7-membered heterocycle, wherein the 5, 6 or 7-membered heterocycle is optionally substituted with one or more (e.g., 1, 2, 3, 4 or 5) Z^{1c} or Z^{1d} groups;

each Rⁿ² is independently selected from H, (C₁-C₈)alkyl, (C₂-C₈)alkenyl, (C₂-C₈)alkynyl, (C₃-C₇)carbocycle, 3-7 membered heterocycle, 5-6 membered monocyclic-heteroaryl, phenyl, halophenyl, 5-6 membered monocyclic-haloheteroaryl, 3-7 membered haloheterocycle, (C₁-C₈)haloalkyl and (C₁-C₈)heteroalkyl;

each R^{p2} is independently selected from (C₁-C₈)alkyl, (C₂-C₈)alkenyl, (C₂-C₈)alkynyl, (C₃-C₇)carbocycle, 3-7 membered heterocycle, 5-6 membered monocyclic-heteroaryl, phenyl, halophenyl, 5-6 membered monocyclic-haloheteroaryl, 3-7 membered haloheterocycle, (C₁-C₈)haloalkyl and (C₁-C₈)heteroalkyl;

R^{q2} and R^{r2} are each independently selected from H, (C₁-C₈)alkyl, (C₂-C₈)alkenyl, (C₂-C₈)alkynyl, (C₃-C₇)carbocycle, 3-7 membered heterocycle, 5-6 membered monocyclic-heteroaryl, phenyl, halophenyl, 5-6 membered monocyclic-haloheteroaryl, 3-7 membered haloheterocycle, (C₁-C₈)haloalkyl and (C₁-C₈)heteroalkyl, or and R^{r2} together with the nitrogen to which they are attached form a 5, 6 or 7-membered heterocycle;

Z² is selected from (C₂-C₈)alkenyl, (C₂-C₈)alkynyl, 6-12 membered aryl, 5-12 membered C-linked-heteroaryl, 3-12 membered C-linked-heterocycle, —C(O)Rⁿ³ and —C(O)NR^{q3}R^{r3}, wherein any 6-12 membered aryl, 5-12 membered C-linked-heteroaryl and 3-12 membered C-linked-heterocycle of Z² is optionally substituted with one or more (e.g., 1, 2, 3, 4 or 5) Z^{2b} or Z^{2c} groups, and wherein any (C₂-C₈)alkenyl and (C₂-C₈)alkynyl of Z² is optionally substituted with one or more (e.g., 1, 2, 3, 4, or 5) Z^{2c} groups;

each Z^{2a} is independently selected from (C₃-C₇)carbocycle, 6-12 membered aryl, 5-12 membered heteroaryl, 3-12 membered heterocycle, halogen, —CN, —ORⁿ⁴, —OC(O)R^{p4}, —OC(O)NR^{q4}R^{r4}, —SRⁿ⁴, —S(O)R^{p4}, —S(O)₂OH, —S(O)₂R^{p4}, —S(O)₂NR^{q4}R^{r4}, —NR^{q4}R^{r4}, —NRⁿ⁴COR^{p4}, —NRⁿ⁴CO₂R^{p4}, —NRⁿ⁴CONR^{q4}R^{r4}, —NRⁿ⁴S(O)₂R^{p4}, —NRⁿ⁴S(O)₂OR^{p4}, —NRⁿ⁴S(O)₂NR^{q4}R^{r4}, NO₂, —C(O)Rⁿ⁴, —C(O)ORⁿ⁴ and —C(O)NR^{q4}R^{r4}, wherein any (C₃-C₇)carbocycle, 6-12 membered aryl, 5-12 membered heteroaryl and 3-12 membered heterocycle of Z^{2a} is optionally substituted with one or more (e.g., 1, 2, 3, 4 or 5) Z^{2b} or Z^{2c} groups;

each Z^{2b} is independently selected from (C₁-C₄)alkyl, (C₁-C₄)heteroalkyl and (C₁-C₄)haloalkyl;

each Z^{2c} is independently selected from halogen, —CN, —ORⁿ⁴, —OC(O)R^{p4}, —OC(O)NR^{q4}R^{r4}, —SRⁿ⁴, —S(O)R^{p4}, —S(O)₂OH, —S(O)₂R^{p4}, —S(O)₂NR^{q4}R^{r4}, —NR^{q4}R^{r4}, —NRⁿ⁴COR^{p4}, —NRⁿ⁴CO₂R^{p4}, —NRⁿ⁴CONR^{q4}R^{r4}, —NRⁿ⁴S(O)₂R^{p4}, —NRⁿ⁴S(O)₂OR^{p4}, —NRⁿ⁴S(O)₂NR^{q4}R^{r4}, NO₂, —C(O)Rⁿ⁴, —C(O)ORⁿ⁴ and —C(O)NR^{q4}R^{r4};

each R^{r3} is independently selected from H, (C₁-C₄)alkyl, (C₂-C₄)alkenyl, (C₃-C₇)carbocycle, 3-12 membered heterocycle, 5-12 membered heteroaryl and 6-12 membered aryl, wherein any (C₃-C₇)carbocycle, 3-12 membered heterocycle, 5-12 membered heteroaryl and 6-12 membered aryl of Rⁿ³ is optionally substituted with one or more (e.g., 1, 2, 3, 4 or 5) Z^{2b} or Z^{2c} groups, and wherein any (C₁-C₄)alkyl, (C₂-C₄)alkenyl and (C₂-C₄)alkynyl of Rⁿ³ is optionally substituted with one or more (e.g., 1, 2, 3, 4 or 5) Z^{2a} groups;

R^{q3} and R^{r3} are each independently selected from H, (C₁-C₄)alkyl, (C₂-C₄)alkenyl, (C₃-C₇)carbocycle, 3-12 membered heterocycle, 5-12 membered heteroaryl and 6-12 membered aryl, wherein any (C₃-C₇)carbocycle, 3-12 membered heterocycle, 5-12 membered heteroaryl and 6-12 membered aryl of R^{q3} or R^{r3} is optionally substituted with one or more (e.g., 1, 2, 3, 4 or 5) Z^{2b} or Z^{2c} groups, and wherein any (C₁-C₄)alkyl and (C₂-C₄)alkenyl of R^{q3} or R^{r3} is optionally substituted with one or more (e.g., 1, 2, 3, 4 or 5) Z^{2a} groups, or R^{q3} and R^{r3} together with the nitrogen to which they are attached form a heterocycle or heteroaryl, wherein the heterocycle or heteroaryl is optionally substituted with one or more (e.g., 1, 2, 3, 4 or 5) Z^{2b} or Z^{2c} groups;

each Rⁿ⁴ is independently selected from H, (C₁-C₄)alkyl, (C₂-C₈)alkenyl, (C₂-C₈)alkynyl, (C₁-C₄)haloalkyl and (C₁-C₄)heteroalkyl;

each R^{p4} is independently selected from (C₁-C₈)alkyl, (C₂-C₄)alkenyl, (C₂-C₄)alkynyl, (C₁-C₄)haloalkyl and (C₁-C₄)heteroalkyl;

R^{q4} and R^{r4} are each independently selected from H, (C₁-C₄)alkyl, (C₂-C₄)alkenyl, (C₂-C₄)alkynyl, (C₁-C₄)haloalkyl and (C₁-C₄)heteroalkyl;

each Z³ is independently selected from halogen, (C₁-C₄)alkyl, —OH, —CN, (C₁-C₄)heteroalkyl and (C₁-C₄)haloalkyl;

each Z⁴ is independently selected from (C₁-C₈)alkyl, (C₂-C₈)alkenyl, (C₂-C₈)alkynyl, (C₃-C₇)carbocycle, halogen, —CN, —ORⁿ⁵, —OC(O)R^{p5}, —OC(O)NR^{q5}R^{r5}, —SRⁿ⁵, —S(O)R^{p5}, —S(O)₂OH, —S(O)₂R^{p5}, —S(O)₂NR^{q5}R^{r5}, —NR^{q5}R^{r5}, —NRⁿ⁵COR^{p5}, —NRⁿ⁵CO₂R^{p5}, —NRⁿ⁵CONR^{q5}R^{r5}, —NRⁿ⁵S(O)₂R^{p5}, —NRⁿ⁵S(O)₂OR^{p5}, —NRⁿ⁵S(O)₂NR^{q5}R^{r5}, NO₂, —C(O)Rⁿ⁵, —C(O)ORⁿ⁵ and —C(O)NR^{q5}R^{r5}, wherein any (C₃-C₇)carbocycle, of Z⁴ is optionally substituted with one or more (e.g., 1, 2, 3, 4 or 5) Z^{4a} or Z^{4b} groups, and wherein any (C₁-C₈)alkyl, (C₂-C₈)alkenyl and (C₂-C₈)alkynyl of Z⁴ is optionally substituted with one or more (e.g., 1, 2, 3, 4 or 5) Z^{4a} groups;

each Z^{4a} is independently selected from halogen, —CN, —ORⁿ⁶, —OC(O)R^{p6}, —OC(O)NR^{q6}R^{r6}, —SRⁿ⁶, —S(O)R^{p6}, —S(O)₂OH, —S(O)₂R^{p6}, —S(O)₂NR^{q6}R^{r6}, —NR^{q6}R^{r6}, —NRⁿ⁶COR^{p6}, —NRⁿ⁶CO₂R^{p6}, —NRⁿ⁶CONR^{q6}R^{r6}, —NRⁿ⁶S(O)₂R^{p6}, —NRⁿ⁶S(O)₂OR^{p6}, —NRⁿ⁶S(O)₂NR^{q6}R^{r6}, NO₂, —C(O)Rⁿ⁶, —C(O)ORⁿ⁶ and —C(O)NR^{q6}R^{r6};

each Z^{4b} is independently selected from (C₁-C₄)alkyl, (C₂-C₄)alkenyl (C₂-C₄)alkynyl and (C₁-C₄)haloalkyl;

each Rⁿ⁵ is independently selected from H, (C₁-C₄)alkyl, (C₁-C₄)haloalkyl, (C₁-C₄)heteroalkyl, (C₂-C₄)alkenyl and (C₂-C₄)alkynyl;

each R^{p5} is independently selected from (C₁-C₄)alkyl, (C₁-C₄)haloalkyl, (C₁-C₄)heteroalkyl, (C₂-C₄)alkenyl and (C₂-C₄)alkynyl;

R^{q5} and R^{r5} are each independently selected from H, (C₁-C₄)alkyl, (C₁-C₄)haloalkyl, (C₁-C₄)heteroalkyl, (C₂-C₄)alkenyl and (C₂-C₄)alkynyl;

each Rⁿ⁶ is independently selected from H, (C₁-C₄)alkyl, (C₁-C₄)haloalkyl, (C₁-C₄)heteroalkyl, (C₂-C₄)alkenyl and (C₂-C₄)alkynyl;

each R^{p6} is independently selected from (C₁-C₄)alkyl, (C₁-C₄)haloalkyl, (C₁-C₄)heteroalkyl, (C₂-C₄)alkenyl and (C₂-C₄)alkynyl;

R^{q6} and R^{r6} are each independently selected from H, (C₁-C₄)alkyl, (C₁-C₄)haloalkyl, (C₁-C₄)heteroalkyl, (C₂-C₄)alkenyl and (C₂-C₄)alkynyl;

each Z⁵ is independently selected from (C₁-C₆)alkyl, halogen, —CN and —ORⁿ⁷, wherein any (C₁-C₆)alkyl of Z⁵ is optionally substituted with one or more (e.g., 1, 2, 3, 4 or 5) halogen; and

each Rⁿ⁷ is independently selected from H, (C₁-C₃)alkyl, (C₁-C₃)haloalkyl and (C₃-C₇)carbocycle;

or a pharmaceutically acceptable salt thereof.

One embodiment provides a pharmaceutical composition comprising a compound of formula I or a pharmaceutically acceptable salt thereof, and a pharmaceutically acceptable carrier.

One embodiment provides a method for treating a Retroviridae viral infection (e.g., an HIV viral infection) in a mammal (e.g., a human), comprising administering a compound of formula I, or a pharmaceutically acceptable salt thereof, to the mammal.

One embodiment provides a method for inhibiting the proliferation of the HIV virus, treating AIDS or delaying the onset of AIDS or ARC symptoms in a mammal (e.g., a human), comprising administering a compound of formula I, or a pharmaceutically acceptable salt thereof, to the mammal.

One embodiment provides a method for treating an HIV infection in a mammal (e.g., a human), comprising administering a compound of formula I, or a pharmaceutically acceptable salt thereof, to the mammal.

One embodiment provides a method for treating an HIV infection in a mammal (e.g., a human), comprising administering to the mammal in need thereof a therapeutically effective amount of a compound of formula I, or a pharmaceutically acceptable salt thereof, in combination with a therapeutically effective amount of one or more additional therapeutic agents selected from the group consisting of HIV protease inhibiting compounds, HIV non-nucleoside inhibitors of reverse transcriptase, HIV nucleoside inhibitors of reverse transcriptase, HIV nucleotide inhibitors of reverse transcriptase, HIV integrase inhibitors, gp41 inhibitors, CXCR4 inhibitors, gp120 inhibitors, CCR5 inhibitors, capsid polymerization inhibitors, and other drugs for treating HIV, and combinations thereof.

One embodiment provides a compound of formula I, or a pharmaceutically acceptable salt thereof for use in medical therapy (e.g., for use in treating a Retroviridae viral infection (e.g., an HIV viral infection) or the proliferation of the HIV virus or AIDS or delaying the onset of AIDS or ARC symptoms in a mammal (e.g., a human)).

One embodiment provides a compound of formula I, or a pharmaceutically acceptable salt thereof for use in the manufacture of a medicament for treating a Retroviridae viral infection (e.g., an HIV viral infection) or the proliferation of the HIV virus or AIDS or delaying the onset of AIDS or ARC symptoms in a mammal (e.g., a human).

One embodiment provides a compound of formula I, or a pharmaceutically acceptable salt thereof, for use in the prophylactic or therapeutic treatment of the proliferation of a Retroviridae virus, an HIV virus or AIDS or for use in the therapeutic treatment of delaying the onset of AIDS or ARC symptoms.

One embodiment provides a compound of formula I, or a pharmaceutically acceptable salt thereof, for use in the prophylactic or therapeutic treatment of a Retroviridae virus infection (e.g., an HIV virus infection).

One embodiment provides the use of a compound of formula I, or a pharmaceutically acceptable salt thereof, for the manufacture of a medicament for a Retroviridae virus infection (e.g., an HIV virus infection) in a mammal (e.g., a human).

One embodiment provides processes and intermediates disclosed herein that are useful for preparing compounds of formula I or salts thereof.

Other embodiments, objects, features and advantages will be set forth in the detailed description of the embodiments that follows, and in part will be apparent from the description, or may be learned by practice, of the claimed invention. These objects and advantages will be realized and attained by the processes and compositions particularly pointed out in the written description and claims hereof. The foregoing Summary has been made with the understanding that it is to be considered as a brief and general synopsis of some of the embodiments disclosed herein, is provided solely for the benefit and convenience of the reader, and is not intended to limit in any manner the scope, or range of equivalents, to which the appended claims are lawfully entitled.

### DETAILED DESCRIPTION

While the present invention is capable of being embodied in various forms, the description below of several embodiments is made with the understanding that the present disclosure is to be considered as an exemplification of the claimed subject matter, and is not intended to limit the appended claims to the specific embodiments illustrated. The headings used throughout this disclosure are provided for convenience only and are not to be construed to limit the claims in any way. Embodiments illustrated under any heading may be combined with embodiments illustrated under any other heading.

### DEFINITIONS

Unless stated otherwise, the following terms and phrases as used herein are intended to have the following meanings:

When trade names are used herein, applicants intend to independently include the tradename product and the active pharmaceutical ingredient(s) of the tradename product.

“Alkyl” is a straight or branched saturated hydrocarbon. For example, an alkyl group can have 1 to 8 carbon atoms (i.e., (C₁-C₈)alkyl) or 1 to 6 carbon atoms (i.e., (C₁-C₆ alkyl) or 1 to 4 carbon atoms (i.e., (C₁-C₄)alkyl). Examples of suitable alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, and octyl (—(CH₂)₇CH₃).

“Alkenyl” is a straight or branched hydrocarbon with at least one carbon-carbon, sp² double bond. For example, an alkenyl group can have 2 to 8 carbon atoms (i.e., C₂-C₈ alkenyl), or 2 to 6 carbon atoms (i.e., C₂-C₆ alkenyl). Examples of suitable alkenyl groups include, but are not limited to, ethylene or vinyl (—CH═CH₂), allyl (—CH₂CH═CH₂) and 5-hexenyl (—CH₂CH₂CH₂CH₂CH═CH₂).

“Alkynyl” is a straight or branched hydrocarbon with at least one carbon-carbon, sp triple bond. For example, an alkynyl group can have 2 to 8 carbon atoms (i.e., C₂-C₈ alkyne,) or 2 to 6 carbon atoms (i.e., C₂-C₆ alkynyl). Examples of suitable alkynyl groups include, but are not limited to, acetylenic (—C≡CH), propargyl (—CH₂C≡CH), and the like.

The term “halo” or “halogen” as used herein refers to fluoro, chloro, bromo and iodo.

The term “haloalkyl” as used herein refers to an alkyl as defined herein, wherein one or more hydrogen atoms of the alkyl are each independently replaced by a halo substituent. For example, (C₁-C₆)haloalkyl is a (C₁-C₆)alkyl wherein one or more of the hydrogen atoms of the (C₁-C₆)alkyl have been replaced by a halo substituent. Examples of haloalkyls include but are not limited to fluoromethyl, fluorochloromethyl, difluoromethyl, difluorochloromethyl, trifluoromethyl, 1,1,1, trifluoroethyl and pentafluoroethyl.

The term “heteroalkyl” as used herein refers to an alkyl as defined herein, wherein one or more of the carbon atoms of the alkyl are replaced by an O, S, or NR^{q}, (or if the carbon atom being replaced is a terminal carbon with an OH, SH or N(R^{q})₂) wherein each R^{q} is independently H or (C₁-C₆)alkyl. For example, (C₁-C₈)heteroalkyl includes a heteroalkyl of one to eight carbons and one or more heteroatoms (e.g., O, S, NR^{q}, OH, SH or N(R^{q})₂). Examples of heteroalkyls include but are not limited to methoxymethyl, ethoxymethyl, methoxy, 2-hydroxyethyl and N,N′-dimethylpropylamine

The term “aryl” as used herein refers to a single all carbon aromatic ring or a multiple condensed all carbon ring system wherein at least one of the rings is aromatic. For example, in certain embodiments, an aryl group has 6 to 20 carbon atoms, 6 to 14 carbon atoms, or 6 to 12 carbon atoms. Aryl includes a phenyl radical. Aryl also includes multiple condensed ring systems (e.g., ring systems comprising 2, 3 or 4 rings) having about 9 to 20 carbon atoms in which at least one ring is aromatic and wherein the other rings may be aromatic or not aromatic (i.e., carbocycle). Such multiple condensed ring systems are optionally substituted with one or more (e.g., 1, 2 or 3) oxo groups on any carbocycle portion of the multiple condensed ring system. The rings of the multiple condensed ring system can be connected to each other via fused, spiro and bridged bonds when allowed by valency requirements. It is to be understood that the point of attachment of a multiple condensed ring system, as defined above, can be at any position of the ring system including an aromatic or a carbocycle portion of the ring. It is also to be understood that when reference is made to a certain atom-range membered aryl (e.g., 6-12 membered aryl), the atom range is for the total ring atoms of the aryl. For example, a 6-membered aryl would include phenyl and a 10-membered aryl would include naphthyl and 1,2,3,4-tetrahydronaphthyl. Non-limiting examples of aryl groups include, but are not limited to, phenyl, indenyl, naphthyl, 1,2,3,4-tetrahydronaphthyl, anthracenyl, and the like.

The term “heteroaryl” as used herein refers to a single aromatic ring that has at least one atom other than carbon in the ring, wherein the atom is selected from the group consisting of oxygen, nitrogen and sulfur; “heteroaryl” also includes multiple condensed ring systems that have at least one such aromatic ring, which multiple condensed ring systems are further described below. Thus, “heteroaryl” includes single aromatic rings of from about 1 to 6 carbon atoms and about 1-4 heteroatoms selected from the group consisting of oxygen, nitrogen and sulfur. The sulfur and nitrogen atoms may also be present in an oxidized form provided the ring is aromatic. Exemplary heteroaryl ring systems include but are not limited to pyridyl, pyrimidinyl, oxazolyl or furyl. “Heteroaryl” also includes multiple condensed ring systems (e.g., ring systems comprising 2, 3 or 4 rings) wherein a heteroaryl group, as defined above, is condensed with one or more rings selected from heteroaryls (to form for example 1,8-naphthyridinyl), heterocycles, (to form for example 1,2,3,4-tetrahydro-1,8-naphthyridinyl), carbocycles (to form for example 5,6,7,8-tetrahydroquinolyl) and aryls (to form for example indazolyl) to form the multiple condensed ring system. Thus, a heteroaryl (a single aromatic ring or multiple condensed ring system) has about 1-20 carbon atoms and about 1-6 heteroatoms within the heteroaryl ring. Such multiple condensed ring systems may be optionally substituted with one or more (e.g., 1, 2, 3 or 4) oxo groups on the carbocycle or heterocycle portions of the condensed ring. The rings of the multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heteroaryl) can be at any position of the multiple condensed ring system including a heteroaryl, heterocycle, aryl or carbocycle portion of the multiple condensed ring system. It is also to be understood that the point of attachment for a heteroaryl or heteroaryl multiple condensed ring system can be at any suitable atom of the heteroaryl or heteroaryl multiple condensed ring system including a carbon atom and a heteroatom (e.g., a nitrogen). It also to be understood that when a reference is made to a certain atom-range membered heteroaryl (e.g., a 5-14 membered heteroaryl), the atom range is for the total ring atoms of the heteroaryl and includes carbon atoms and heteroatoms. For example, a 5-membered heteroaryl would include a thiazolyl and a 10-membered heteroaryl would include a quinolinyl. Exemplary heteroaryls include but are not limited to pyridyl, pyrrolyl, pyrazinyl, pyrimidinyl, pyridazinyl, pyrazolyl, thienyl, indolyl, imidazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, quinazolyl, 5,6,7,8-tetrahydroisoquinolinyl benzofuranyl, benzimidazolyl, thianaphthenyl, pyrrolo[2,3-b]pyridinyl, quinazolinyl-4(3H)-one, triazolyl, 4,5,6,7-tetrahydro-1H-indazole and 3b,4,4a,5-tetrahydro-1H-cyclopropa[3,4]cyclopenta[1,2-c]pyrazole.

The term “C-linked-heteroaryl” (carbon-linked heteroaryl) as used herein refers to a heteroaryl that is linked at a carbon atom of the heteroaryl to the remainder of the compound of formula I (e.g., a C-linked-heteroaryl of Z² bonded to the A ring of formula I through a carbon atom of the C-linked-heteroaryl).

The term “heterocyclyl” or “heterocycle” as used herein refers to a single saturated or partially unsaturated ring that has at least one atom other than carbon in the ring, wherein the atom is selected from the group consisting of oxygen, nitrogen and sulfur; the term also includes multiple condensed ring systems that have at least one such saturated or partially unsaturated ring, which multiple condensed ring systems are further described below. Thus, the term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The ring may be substituted with one or more (e.g., 1, 2 or 3) oxo groups and the sulfur and nitrogen atoms may also be present in their oxidized forms. Exemplary heterocycles include but are not limited to azetidinyl, tetrahydrofuranyl and piperidinyl. The term “heterocycle” also includes multiple condensed ring systems (e.g., ring systems comprising 2, 3 or 4 rings) wherein a single heterocycle ring (as defined above) can be condensed with one or more groups selected from heterocycles (to form for example a 1,8-decahydronapthyridinyl), carbocycles (to form for example a decahydroquinolyl) and aryls to form the multiple condensed ring system. Thus, a heterocycle (a single saturated or single partially unsaturated ring or multiple condensed ring system) has about 2-20 carbon atoms and 1-6 heteroatoms within the heterocycle ring. Such multiple condensed ring systems may be optionally substituted with one or more (e.g., 1, 2, 3 or 4) oxo groups on the carbocycle or heterocycle portions of the multiple condensed ring. The rings of the multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heterocycle) can be at any position of the multiple condensed ring system including a heterocycle, aryl and carbocycle portion of the ring. It is also to be understood that the point of attachment for a heterocycle or heterocycle multiple condensed ring system can be at any suitable atom of the heterocycle or heterocycle multiple condensed ring system including a carbon atom and a heteroatom (e.g., a nitrogen). It is also to be understood that when reference is made to a certain atom-range membered heterocycle (e.g., a 3-14 membered heterocycle), the atom range is for the total ring atoms of the heterocycle and includes carbon atoms and heteroatoms. For example, a 3-membered heterocycle would include an aziridinyl and a 10-membered heterocycle would include a 1,2,3,4-tetrahydroquinolyl. Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl, 1,4-benzodioxanyl, spiro[cyclopropane-1,1′-isoindolinyl]-3′-one, isoindolinyl-1-one, 2-oxa-6-azaspiro[3.3]heptanyl, imidazolidin-2-one and pyrrolidin-2-one.

The term “C-linked-heterocycle” (carbon-linked heterocycle) as used herein refers to a “heterocycle that is linked at a carbon atom of the heterocycle to the remainder of the compound of formula I (e.g., a C-linked-heterocycle of Z² bonded to the A ring of formula I through a carbon atom of the C-linked-heterocycle).

The term “carbocycle” or “carbocyclyl” refers to a single saturated (i.e., cycloalkyl) or a single partially unsaturated (e.g., cycloalkenyl, cycloalkadienyl, etc.) all carbon ring having 3 to 7 carbon atoms (i.e., (C₃-C₇)carbocycle). The term “carbocycle” or “carbocyclyl” also includes multiple condensed, saturated and partially unsaturated all carbon ring systems (e.g., ring systems comprising 2, 3 or 4 carbocyclic rings). Accordingly, carbocycle includes multicyclic carbocyles such as a bicyclic carbocycles (e.g., bicyclic carbocycles having about 6 to 12 carbon atoms such as bicyclo[3.1.0]hexane and bicyclo[2.1.1]hexane), and polycyclic carbocycles (e.g tricyclic and tetracyclic carbocycles with up to about 20 carbon atoms). The rings of the multiple condensed ring system can be connected to each other via fused, spiro and bridged bonds when allowed by valency requirements. For example, multicyclic carbocyles can be connected to each other via a single carbon atom to form a spiro connection (e.g., spiropentane, spiro[4,5]decane, etc), via two adjacent carbon atoms to form a fused connection (e.g., carbocycles such as decahydronaphthalene, norsabinane, norcarane) or via two non-adjacent carbon atoms to form a bridged connection (e.g., norbornane, bicyclo[2.2.2]octane, etc). The “carbocycle” or “carbocyclyl” can also be optionally substituted with one or more (e.g., 1, 2 or 3) oxo groups. Non-limiting examples of monocyclic carbocycles include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl and 1-cyclohex-3-enyl.

The term “halophenyl” as used herein refers to phenyl, wherein one or more (e.g., 1, 2, 3, 4 or 5) hydrogen atoms of the phenyl are each replaced independently by a halo substituent. Examples of halophenyl include but are not limited to fluorophenyl, 2,3-dichlorophenyl, 3-bromo-4-fluorophenyl and pentafluorophenyl.

The term “haloheteroaryl” as used herein refers to a heteroaryl, wherein one or more (e.g., 1, 2, 3, 4 or 5) hydrogen atoms of the heteroaryl are each replaced independently by a halo substituent. Examples of haloheteroaryl include but are not limited to 2-fluorofuryl, 2,3-dichloropyridinyl and 8-chloro-3-fluoroquinolinyl.

The term “haloheterocycle” as used herein refers to a heterocycle, wherein one or more (e.g., 1, 2, 3, 4 or 5) hydrogen atoms of the heterocycle are each replaced independently by a halo substituent. Examples of haloheteroaryl include but are not limited to 2-fluoropiperidinyl, 2-chloro-3-fluoropiperazinyl and 3-bromopyrrolidinyl.

One skilled in the art will recognize that substituents and other moieties of the compounds of formula I should be selected in order to provide a compound which is sufficiently stable to provide a pharmaceutically useful compound which can be formulated into an acceptably stable pharmaceutical composition. Compounds of formula I which have such stability are contemplated as falling within the scope of the present invention.

The modifier “about” used in connection with a quantity is inclusive of the stated value and has the meaning dictated by the context (e.g., includes the degree of error associated with measurement of the particular quantity). The word “about” may also be represented symbolically by “˜” in the context of a chemical measurement (e.g., ˜50 mg or pH ˜7).

The term “treatment” or “treating,” to the extent it relates to a disease or condition includes preventing the disease or condition from occurring, inhibiting the disease or condition, eliminating the disease or condition, and/or relieving one or more symptoms of the disease or condition.

### Stereoisomers

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York.

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers or axes of chirality and whose molecules are not mirror images of one another. Diastereomers typically have different physical properties, e.g., melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

The compounds disclosed herein may have chiral centers, e.g., chiral carbon atoms. Such compounds thus include racemic mixtures of all stereoisomers, including enantiomers, diastereomers, and atropisomers. In addition, the compounds disclosed herein include enriched or resolved optical isomers at any or all asymmetric, chiral atoms. In other words, the chiral centers apparent from the depictions are provided as the chiral isomers or racemic mixtures. Both racemic and diastereomeric mixtures, as well as the individual optical isomers isolated or synthesized, substantially free of their enantiomeric or diastereomeric partners, are all within the scope of the invention. The racemic mixtures can be separated into their individual, substantially optically pure isomers through well-known techniques such as, for example, the separation of diastereomeric salts formed with optically active adjuncts, e.g., acids or bases followed by conversion back to the optically active substances. The desired optical isomer can also be synthesized by means of stereospecific reactions, beginning with the appropriate stereoisomer of the desired starting material.

It is to be understood that for compounds disclosed herein when a bond is drawn in a non-stereochemical manner (e.g., flat) the atom to which the bond is attached includes all stereochemical possibilities. It is also to be understood that when a bond is drawn in a stereochemical manner (e.g., bold, bold-wedge, dashed or dashed-wedge) the atom to which the stereochemical bond is attached has the stereochemistry as shown unless otherwise noted. Accordingly, in one embodiment, a compound disclosed herein is greater than 50% a single enantiomer. In another embodiment, a compound disclosed herein is at least 80% a single enantiomer. In another embodiment, a compound disclosed herein is at least 90% a single enantiomer. In another embodiment, a compound disclosed herein is at least 98% a single enantiomer. In another embodiment, a compound disclosed herein is at least 99% a single enantiomer. In another embodiment, a compound disclosed herein is greater than 50% a single diastereomer. In another embodiment, a compound disclosed herein is at least 80% a single diastereomer. In another embodiment, a compound disclosed herein is at least 90% a single diastereomer. In another embodiment, a compound disclosed herein is at least 98% a single diastereomer. In another embodiment, a compound disclosed herein is at least 99% a single diastereomer.

### Tautomers

The compounds disclosed herein can also exist as tautomeric isomers in certain cases.

Although only one delocalized resonance structure may be depicted, all such forms are contemplated within the scope of the invention. For example, ene-amine tautomers can exist for purine, pyrimidine, imidazole, guanidine, amidine, and tetrazole systems and all their possible tautomeric forms are within the scope of the invention. Another non-limiting example includes keto-enol tautomers of heteroaryls. Such tautomers are exemplified by T1/T1′, T2/T2′ and T3/T3′. All such tautomeric forms are also within the scope of the invention.

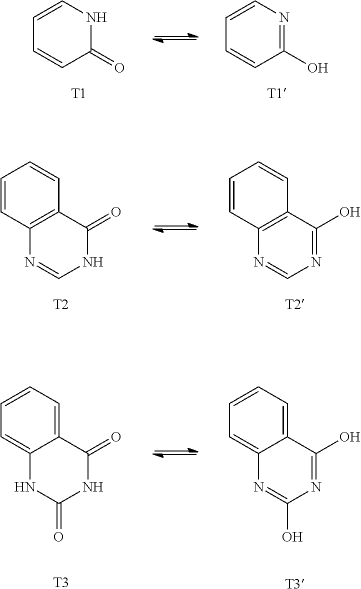

### Protecting Groups

“Protecting group” refers to a moiety of a compound that masks or alters the properties of a functional group or the properties of the compound as a whole. Chemical protecting groups and strategies for protection/deprotection are well known in the art. See e.g., Protective Groups in Organic Chemistry, Theodora W. Greene, John Wiley & Sons, Inc., New York, 1991. Protecting groups are often utilized to mask the reactivity of certain functional groups, to assist in the efficiency of desired chemical reactions, e.g., making and breaking chemical bonds in an ordered and planned fashion. Protection of functional groups of a compound alters other physical properties besides the reactivity of the protected functional group, such as the polarity, lipophilicity (hydrophobicity), and other properties which can be measured by common analytical tools. Chemically protected intermediates may themselves be biologically active or inactive.

### Salts and Hydrates

Examples of pharmaceutically acceptable salts of the compounds disclosed herein include salts derived from an appropriate base, such as an alkali metal (for example, sodium), an alkaline earth metal (for example, magnesium), ammonium and NX₄⁺ (wherein X is C₁-C₄ alkyl). Pharmaceutically acceptable salts of a nitrogen atom or an amino group include for example salts of organic carboxylic acids such as acetic, benzoic, lactic, fumaric, tartaric, maleic, malonic, malic, isethionic, lactobionic and succinic acids; organic sulfonic acids, such as methanesulfonic, ethanesulfonic, benzenesulfonic and p-toluenesulfonic acids; and inorganic acids, such as hydrochloric, hydrobromic, sulfuric, phosphoric and sulfamic acids. Pharmaceutically acceptable salts of a compound of a hydroxy group include the anion of said compound in combination with a suitable cation such as Na⁺ and NX₄⁺ (wherein each X is independently selected from H or a C₁-C₄ alkyl group).

For therapeutic use, salts of active ingredients of the compounds disclosed herein will typically be pharmaceutically acceptable, i.e., they will be salts derived from a physiologically acceptable acid or base. However, salts of acids or bases which are not pharmaceutically acceptable may also find use, for example, in the preparation or purification of a compound of formula I or another compound disclosed herein. All salts, whether or not derived from a physiologically acceptable acid or base, are within the scope of the present invention.

Metal salts typically are prepared by reacting the metal hydroxide with a compound disclosed herein. Examples of metal salts which are prepared in this way are salts containing Li⁺, Na⁺, and K⁺. A less soluble metal salt can be precipitated from the solution of a more soluble salt by addition of the suitable metal compound.

In addition, salts may be formed from acid addition of certain organic and inorganic acids, e.g., HCl, HBr, H₂SO₄, H₃PO₄ or organic sulfonic acids, to basic centers, such as amines. Finally, it is to be understood that the compositions herein comprise compounds disclosed herein in their un-ionized, as well as zwitterionic form, and combinations with water as in hydrates.

### Isotopes

It is understood by one skilled in the art that this invention also includes any compound claimed that may be enriched at any or all atoms above naturally occurring isotopic ratios with one or more isotopes such as, but not limited to, deuterium (²H or D). As a non-limiting example, in certain embodiments, a —CH₃ group is replaced with —CD₃.

Specific values listed below for radicals, substituents, and ranges in the embodiments of the invention are for illustration only; they do not exclude other defined values or other values within defined ranges for the radicals and substituents.

### Compounds of Formula I.

A specific group of compounds of formula I are compounds of formula Ia.

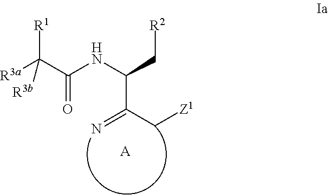

or a pharmaceutically acceptable salt thereof.

Another specific group of compounds of formula I are compounds of formula Ib.

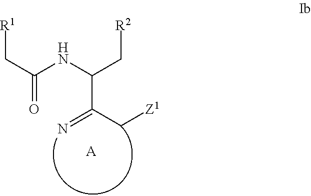

or a pharmaceutically acceptable thereof.

Another specific group of compounds of formula I are compounds of formula Ic.

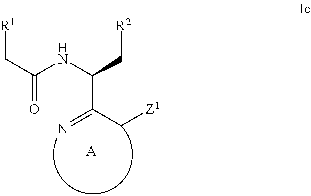

or a pharmaceutically acceptable thereof.

Another specific group of compounds of formula I are compounds of formula Id.

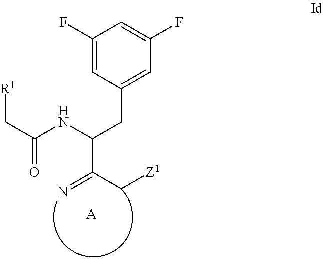

or a pharmaceutically acceptable thereof.

Another specific group of compounds of formula I are compounds of formula Ie.

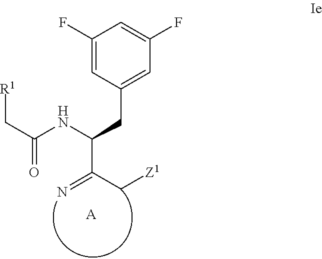

or a pharmaceutically acceptable thereof.

Another specific group of compounds of formula I are compounds of formula If.

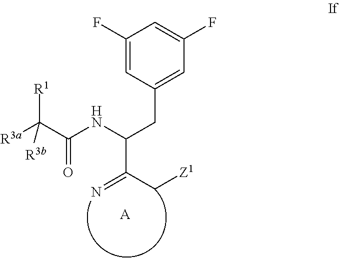

or a pharmaceutically acceptable thereof.

Another specific group of compounds of formula I are compounds of formula Ig.

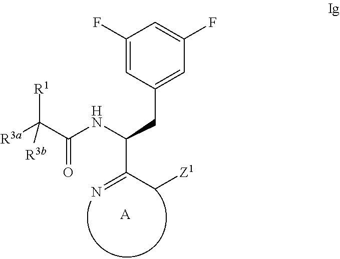

or a pharmaceutically acceptable thereof.

Specific values listed below are values for compounds of formula I as well as all related formulas (e.g., formulas Ia, Ib, Ic, Id, Ie, If, Ig). It is to be understood that two or more values may combined.

A specific group of compounds of formula I are compounds wherein each R^{3a} and R^{3b} is independently selected from H, halogen, (C₁-C₃)alkyl, and (C₁-C₃)haloalkyl.

A specific group of compounds of formula I are compounds wherein each R^{3a} and R^{3b} is independently selected from H, (C₁-C₃)alkyl, and (C₁-C₃)haloalkyl.

A specific group of compounds of formula I are compounds wherein each R^{3a} and R^{3b} is independently selected from H and (C₁-C₃)alkyl.

A specific group of compounds of formula I are compounds wherein each R^{3a} and R^{3b} is independently selected from H, methyl and ethyl.

A specific group of compounds of formula I are compounds wherein each R^{3a} and R^{3b} is independently selected from H and methyl.

A specific group of compounds of formula I are compounds wherein R^{3a} is H and R^{3b} is (C₁-C₃)alkyl.

A specific group of compounds of formula I are compounds wherein R^{3a} is H and R^{3b} is methyl or ethyl.

A specific group of compounds of formula I are compounds wherein R^{3a} is H and R^{3b} is methyl.

A specific value for R^{3a} and R^{3b} is H.

A specific value for R² is phenyl or a 5-membered monocyclic-heteroaryl, wherein any phenyl or 5-membered monocyclic-heteroaryl of R² is optionally substituted with one or more (e.g., 1, 2, 3, 4 or 5) Z⁵ groups.

A specific value for R² is phenyl or a 5-membered monocyclic-heteroaryl, wherein any phenyl or 5-membered monocyclic-heteroaryl of R² is substituted with one or more (e.g., 1, 2, 3, 4 or 5) Z⁵ groups.

A specific value for R² is phenyl optionally substituted with one or more (e.g., 1, 2, 3, 4 or 5) Z⁵ groups.

A specific value for R² is phenyl substituted with one or more (e.g., 1, 2, 3, 4 or 5) Z⁵ groups.

A specific value for Z⁵ is halogen.

A specific value for Z⁵ is fluoro.

A specific value for R² is 3,5-difluorophenyl.

A specific value for A is pyridinyl, pyrimidinyl, pyrazinyl or pyridazinyl, wherein any pyridinyl, pyrimidinyl, pyrazinyl or pyridazinyl of A is substituted with one Z¹ group at the position shown, one Z² group and optionally substituted with one or more (e.g., 1 or 2) Z³ groups.

A specific value for A is pyridinyl, pyrimidinyl, pyrazinyl or pyridazinyl, wherein any pyridinyl, pyrimidinyl, pyrazinyl or pyridazinyl of A is substituted with one Z¹ group at the position shown and one Z² group.

A specific value for A is pyridinyl, wherein any pyridinyl of A is substituted with one Z¹ group at the position shown, one Z² group, and optionally substituted with one or more (e.g., 1 or 2) Z³ groups.

A specific value for A is pyridinyl, wherein any pyridinyl of A is substituted with one Z¹ group at the position shown and one Z² group

A specific value for A is selected from:

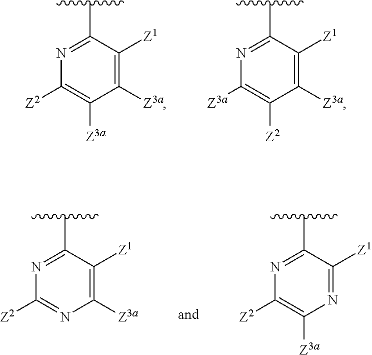

wherein each Z^{3a} is independently selected from H and Z³.

A specific value for A is selected from:

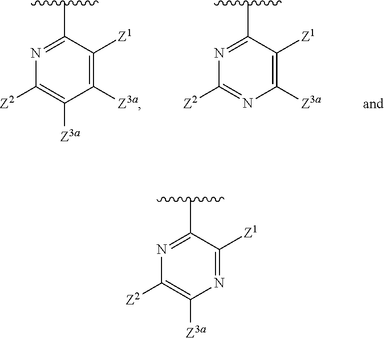

wherein each Z^{3a} is independently selected from H and Z³.

A specific value for A is selected from:

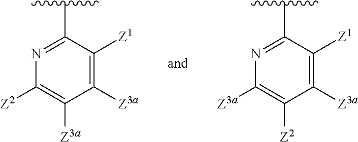

wherein each Z^{3a} is independently selected from H and Z³.

A specific value for A is:

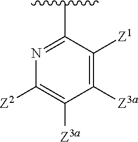

wherein each Z^{3a} is independently selected from H and Z³.

A specific value for A is:

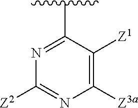

wherein each Z^{3a} is independently selected from H and Z³.

A specific value for A is:

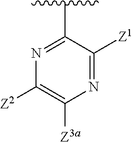

wherein each Z^{3a} is independently selected from H and Z³.

A specific value for Z^{3a} is H.

A specific value for Z¹ is selected from phenyl, 5-14 membered heteroaryl and 3-14 membered heterocycle, wherein any phenyl, 5-14 membered heteroaryl and 3-14 membered heterocycle of Z¹ is optionally substituted with one or more (e.g., 1, 2, 3, 4 or 5) Z^{1a} or Z^{1b} groups.

A specific value for Z¹ is selected from phenyl, 5-12 membered heteroaryl and 3-12 membered heterocycle, wherein any phenyl, 5-12 membered heteroaryl and 3-12 membered heterocycle of Z¹ is optionally substituted with one or more (e.g., 1, 2, 3, 4 or 5) Z^{1a} or Z^{1b} groups.

A specific value for Z¹ is selected from phenyl, 5-14 membered heteroaryl and 3-14 membered heterocycle, wherein any phenyl, 5-14 membered heteroaryl and 3-14 membered heterocycle of Z¹ is optionally substituted with one or more (e.g., 1, 2, 3, 4 or 5) Z^{1a} groups.

A specific value for Z¹ is selected from phenyl, 5-12 membered heteroaryl and 3-12 membered heterocycle, wherein any phenyl, 5-12 membered heteroaryl and 3-12 membered heterocycle of Z¹ is optionally substituted with one or more (e.g., 1, 2, 3, 4 or 5) Z^{1a} groups.

A specific value for Z¹ is selected from phenyl, 5-6 membered monocyclic-heteroaryl, 8-10 membered bicyclic-heteroaryl, 8-10 membered bicyclic-heterocycle and 9-12 membered tricyclic-heterocycle wherein any phenyl, 5-6 membered monocyclic-heteroaryl, 8-10 membered bicyclic-heteroaryl, 8-10 membered bicyclic-heterocycle and 9-12 membered tricyclic-heterocycle of Z¹ is optionally substituted with one or more (e.g., 1, 2, 3, 4 or 5) Z^{1a} or Z^{1b} groups.

A specific value for Z¹ is selected from phenyl, 5-6 membered monocyclic-heteroaryl, 8-10 membered bicyclic-heteroaryl, 8-10 membered bicyclic-heterocycle and 9-12 membered tricyclic-heterocycle wherein any phenyl, 5-6 membered monocyclic-heteroaryl, 8-10 membered bicyclic-heteroaryl, 8-10 membered bicyclic-heterocycle and 9-12 membered tricyclic-heterocycle of Z¹ is optionally substituted with one or more (e.g., 1, 2, 3, 4 or 5) Z^{1a} groups.

A specific value for Z¹ is selected from phenyl, 5-6 membered monocyclic-heteroaryl, 8-10 membered bicyclic-heteroaryl, 8-10 membered bicyclic-heterocycle and 9-12 membered tricyclic-heterocycle, wherein the 5-6 membered monocyclic-heteroaryl, 8-10 membered bicyclic-heteroaryl, 8-10 membered bicyclic-heterocycle and 9-12 membered tricyclic-heterocycle have 1-11 carbon atoms and 1-5 heteroatoms in the ring system, and wherein any phenyl, 5-6 membered monocyclic-heteroaryl, 8-10 membered bicyclic-heteroaryl, 8-10 membered bicyclic-heterocycle and 9-12 membered tricyclic-heterocycle of Z¹ is optionally substituted with one or more (e.g., 1, 2, 3, 4 or 5) Z^{1a} or Z^{1b} groups.

A specific value for Z¹ is selected from phenyl, 5-6 membered monocyclic-heteroaryl, 8-10 membered bicyclic-heteroaryl, 8-10 membered bicyclic-heterocycle and 9-12 membered tricyclic-heterocycle, wherein the 5-6 membered monocyclic-heteroaryl, 8-10 membered bicyclic-heteroaryl, 8-10 membered bicyclic-heterocycle and 9-12 membered tricyclic-heterocycle have 1-11 carbon atoms and 1-5 heteroatoms in the ring system, and wherein any phenyl, 5-6 membered monocyclic-heteroaryl, 8-10 membered bicyclic-heteroaryl, 8-10 membered bicyclic-heterocycle and 9-12 membered tricyclic-heterocycle of Z¹ is optionally substituted with one or more (e.g., 1, 2, 3, 4 or 5) Z^{1a} groups.

A specific value for Z¹ is selected from phenyl, 5-6 membered monocyclic-heteroaryl, 8-10 membered bicyclic-heteroaryl, 8-10 membered bicyclic-heterocycle and 9-12 membered tricyclic-heterocycle, wherein the 5-6 membered monocyclic-heteroaryl, 8-10 membered bicyclic-heteroaryl, 8-10 membered bicyclic-heterocycle and 9-12 membered tricyclic-heterocycle have 4-11 carbon atoms and 1-3 heteroatoms in the ring system, and wherein any phenyl, 5-6 membered monocyclic-heteroaryl, 8-10 membered bicyclic-heteroaryl, 8-10 membered bicyclic-heterocycle and 9-12 membered tricyclic-heterocycle of Z¹ is optionally substituted with one or more (e.g., 1, 2, 3, 4 or 5) Z^{1a} or Z^{1b} groups.

A specific value for Z¹ is selected from phenyl, 5-6 membered monocyclic-heteroaryl, 8-10 membered bicyclic-heteroaryl, 8-10 membered bicyclic-heterocycle and 9-12 membered tricyclic-heterocycle, wherein the 5-6 membered monocyclic-heteroaryl, 8-10 membered bicyclic-heteroaryl, 8-10 membered bicyclic-heterocycle and 9-12 membered tricyclic-heterocycle have 4-11 carbon atoms and 1-3 heteroatoms in the ring system, and wherein any phenyl, 5-6 membered monocyclic-heteroaryl, 8-10 membered bicyclic-heteroaryl, 8-10 membered bicyclic-heterocycle and 9-12 membered tricyclic-heterocycle of Z¹ is optionally substituted with one or more (e.g., 1, 2, 3, 4 or 5) Z^{1a} groups.

A specific value for Z¹ is selected from 8-10 membered bicyclic-heteroaryl and 8-10 membered bicyclic-heterocycle, wherein any from 8-10 membered bicyclic-heteroaryl and 8-10 membered bicyclic-heterocycle of Z¹ is optionally substituted with one or more Z^{1a} or Z^{1b} groups.

A specific value for Z¹ is selected from 8-10 membered bicyclic-heteroaryl and 8-10 membered bicyclic-heterocycle, wherein any from 8-10 membered bicyclic-heteroaryl and 8-10 membered bicyclic-heterocycle of Z¹ is optionally substituted with one or more.

A specific value for Z¹ is selected from 8-10 membered bicyclic-heteroaryl and 8-10 membered bicyclic-heterocycle, wherein the 8-10 membered bicyclic-heteroaryl and 8-10 membered bicyclic-heterocycle have 3-9 carbon atoms and 1-5 heteroatoms in the ring system, and wherein any 8-10 membered bicyclic-heteroaryl and 8-10 membered bicyclic-heterocycle of Z¹ is optionally substituted with one or more Z^{1a} or Z^{1b} groups.

A specific value for Z¹ is selected from 8-10 membered bicyclic-heteroaryl and 8-10 membered bicyclic-heterocycle, wherein the 8-10 membered bicyclic-heteroaryl and 8-10 membered bicyclic-heterocycle have 3-9 carbon atoms and 1-5 heteroatoms in the ring system, and wherein any 8-10 membered bicyclic-heteroaryl and 8-10 membered bicyclic-heterocycle of Z¹ is optionally substituted with one or more Z^{1a} groups.

A specific value for Z¹ is selected from phenyl, 1H-pyrrolo[2,3-b]pyridinyl, 1-oxoisoindolinyl, 4-oxo-3,4-dihydroquinazolinyl, 3-oxospiro[cyclopropane-1,1′-isoindolin]-yl, 1H-2-oxo-pyridinyl and 2,4-dioxo-1,2,3,4-tetrahydrorquinazolinyl, wherein any phenyl, 1H-pyrrolo[2,3-b]pyridinyl, 1-oxoisoindolinyl, 4-oxo-3,4-dihydroquinazolinyl, 3-oxospiro[cyclopropane-1,1′-isoindolin]-yl, 1H-2-oxo-pyridinyl and 2,4-dioxo-1,2,3,4-tetrahydrorquinazolinyl of Z¹ is optionally substituted with one or more (e.g., 1, 2, 3, 4 or 5) Z^{1a} or Z^{1b} groups.

A specific value for Z¹ is selected from phenyl, 1H-pyrrolo[2,3-b]pyridinyl, 1-oxoisoindolinyl, 4-oxo-3,4-dihydroquinazolinyl, 3-oxospiro[cyclopropane-1,1′-isoindolin]-yl, 1H-2-oxo-pyridinyl and 2,4-dioxo-1,2,3,4-tetrahydrorquinazolinyl as shown by the following formulas;

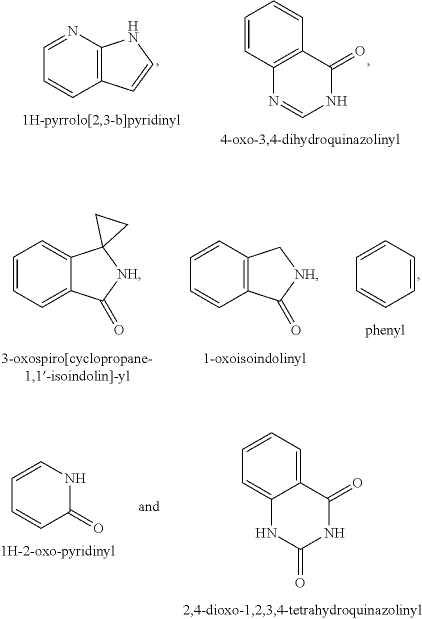

wherein any phenyl, 1H-pyrrolo[2,3-b]pyridinyl, 1-oxoisoindolinyl, 4-oxo-3,4-dihydroquinazolinyl, 3-oxospiro[cyclopropane-1,1′-isoindolin]-yl, 1H-2-oxo-pyridinyl and 2,4-dioxo-1,2,3,4-tetrahydrorquinazolinyl of Z¹ is optionally substituted with one or more (e.g., 1, 2, 3, 4 or 5) Z^{1a} or Z^{1b} groups.

A specific value for Z¹ is selected from phenyl, 1H-pyrrolo[2,3-b]pyridinyl, 1-oxoisoindolinyl, 3-oxospiro[cyclopropane-1,1′-isoindolin]-yl, pyridinyl and quinazolinyl, wherein any phenyl, 1H-pyrrolo[2,3-b]pyridinyl, 1-oxoisoindolinyl, 3-oxospiro[cyclopropane-1,1′-isoindolin]-yl, pyridinyl and quinazolinyl of Z¹ is optionally substituted with one or more (e.g., 1, 2, 3, 4 or 5) Z^{1a} or Z^{1b} groups.

A specific value for Z¹ is selected from phenyl, 1H-pyrrolo[2,3-b]pyridinyl, 1-oxoisoindolinyl, 4-oxo-3,4-dihydroquinazolinyl, 3-oxospiro[cyclopropane-1,1′-isoindolin]-yl, 1H-2-oxo-pyridinyl and 2,4-dioxo-1,2,3,4-tetrahydrorquinazolinyl, wherein any phenyl, 1H-pyrrolo[2,3-b]pyridinyl, 1-oxoisoindolinyl, 4-oxo-3,4-dihydroquinazolinyl, 3-oxospiro[cyclopropane-1,1′-isoindolin]-yl, 1H-2-oxo-pyridinyl and 2,4-dioxo-1,2,3,4-tetrahydrorquinazolinyl of Z¹ is optionally substituted with one or more (e.g., 1, 2, 3, 4 or 5) Z^{1a} groups.

A specific value for Z¹ is selected from phenyl, 1H-pyrrolo[2,3-b]pyridinyl, 1-oxoisoindolinyl, 3-oxospiro[cyclopropane-1,1′-isoindolin]-yl, pyridinyl and quinazolinyl, wherein any phenyl, 1H-pyrrolo[2,3-b]pyridinyl, 1-oxoisoindolinyl, 3-oxospiro[cyclopropane-1,1′-isoindolin]-yl, pyridinyl and quinazolinyl of Z¹ is optionally substituted with one or more (e.g., 1, 2, 3, 4 or 5) Z^{1a} groups.

A specific value for Z¹ is selected from phenyl, 1H-pyrrolo[2,3-b]pyridin-5-yl, 1-oxoisoindolin-5-yl, 1-oxoisoindolin-4-yl, 4-oxo-3,4-dihydroquinazolin-8-yl, 3′-oxospiro[cyclopropane-1,1′-isoindolin]-5′-yl, 1H-2-oxo-pyridin-4-yl and 2,4-dioxo-1,2,3,4-tetrahydrorquinazolin-8-yl, wherein any phenyl, 1H-pyrrolo[2,3-b]pyridin-5-yl, 1-oxoisoindolin-5-yl, 1-oxoisoindolin-4-yl, 4-oxo-3,4-dihydroquinazolin-8-yl, 3′-oxospiro[cyclopropane-1,1′-isoindolin]-5′-yl, 1H-2-oxo-pyridin-4-yl and 2,4-dioxo-1,2,3,4-tetrahydrorquinazolin-8-yl of Z¹ is optionally substituted with one or more (e.g., 1, 2, 3, 4 or 5) Z^{1a} or Z^{1b} groups.

A specific value for Z¹ is selected from phenyl, 1H-pyrrolo[2,3-b]pyridin-5-yl, 1-oxoisoindolin-5-yl, 1-oxoisoindolin-4-yl, 3′-oxospiro[cyclopropane-1,1′-isoindolin]-5′-yl, pyridin-4-yl and quinazolin-8-yl, wherein any phenyl, 1H-pyrrolo[2,3-b]pyridin-5-yl, 1-oxoisoindolin-5-yl, 1-oxoisoindolin-4-yl, 3′-oxospiro[cyclopropane-1,1′-isoindolin]-5′-yl, pyridin-4-yl and quinazolin-8-yl of Z¹ is optionally substituted with one or more (e.g., 1, 2, 3, 4 or 5) Z^{1a} or Z^{1b} groups.

A specific value for Z¹ is selected from phenyl, 1H-pyrrolo[2,3-b]pyridin-5-yl, 1-oxoisoindolin-5-yl, 1-oxoisoindolin-4-yl, 4-oxo-3,4-dihydroquinazolin-8-yl, 3′-oxospiro[cyclopropane-1,1′-isoindolin]-5′-yl, 1H-2-oxo-pyridin-4-yl and 2,4-dioxo-1,2,3,4-tetrahydrorquinazolin-8-yl as shown by the following formulas;

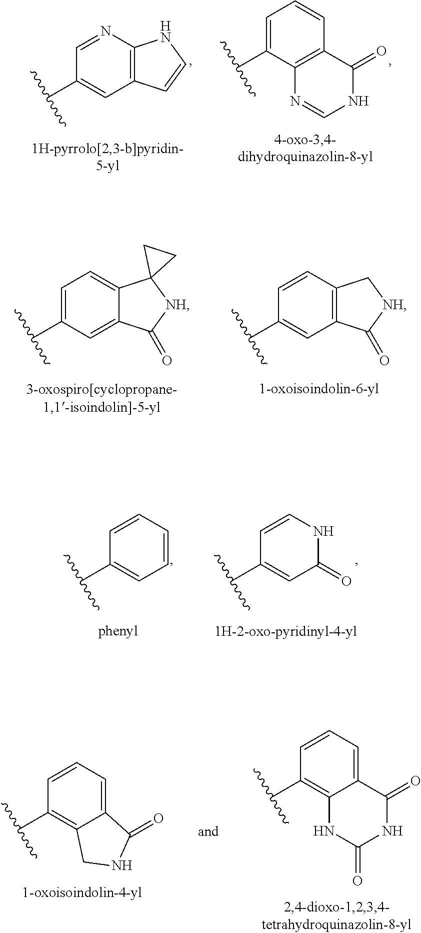

wherein any phenyl, 1H-pyrrolo[2,3-b]pyridin-5-yl, 1-oxoisoindolin-5-yl, 1-oxoisoindolin-4-yl, 4-oxo-3,4-dihydroquinazolin-8-yl, 3′-oxospiro[cyclopropane-1,1′-isoindolin]-5′-yl, 1H-2-oxo-pyridin-4-yl and 2,4-dioxo-1,2,3,4-tetrahydrorquinazolin-8-yl of Z¹ is optionally substituted with one or more (e.g., 1, 2, 3, 4 or 5) Z^{1a} or Z^{1b} groups.

A specific value for Z¹ is selected from phenyl, 1H-pyrrolo[2,3-b]pyridin-5-yl, 1-oxoisoindolin-5-yl, 1-oxoisoindolin-4-yl, 4-oxo-3,4-dihydroquinazolin-8-yl, 3′-oxospiro[cyclopropane-1,1′-isoindolin]-5′-yl, 1H-2-oxo-pyridin-4-yl and 2,4-dioxo-1,2,3,4-tetrahydrorquinazolin-8-yl, wherein any phenyl, 1H-pyrrolo[2,3-b]pyridin-5-yl, 1-oxoisoindolin-5-yl, 1-oxoisoindolin-4-yl, 4-oxo-3,4-dihydroquinazolin-8-yl, 3′-oxospiro[cyclopropane-1,1′-isoindolin]-5′-yl, 1H-2-oxo-pyridin-4-yl and 2,4-dioxo-1,2,3,4-tetrahydrorquinazolin-8-yl of Z¹ is optionally substituted with one or more (e.g., 1, 2, 3, 4 or 5) Z^{1a} groups.

A specific value for Z¹ is selected from phenyl, 1H-pyrrolo[2,3-b]pyridin-5-yl, 1-oxoisoindolin-5-yl, 1-oxoisoindolin-4-yl, 3′-oxospiro[cyclopropane-1,1′-isoindolin]-5′-yl, pyridin-4-yl and quinazolin-8-yl, wherein any phenyl, 1H-pyrrolo[2,3-b]pyridin-5-yl, 1-oxoisoindolin-5-yl, 1-oxoisoindolin-4-yl, 3′-oxospiro[cyclopropane-1,1′-isoindolin]-5′-yl, pyridin-4-yl and quinazolin-8-yl of Z¹ is optionally substituted with one or more (e.g., 1, 2, 3, 4 or 5) Z^{1a} groups.

A specific group of compounds of formula I are compounds wherein Z¹ is not substituted with Z^{1b}

A specific value for each Z^{1a} is independently selected from halogen, —ORⁿ¹ and —C(O)NR^{q1}R^{r1}.

A specific value for each Z^{1a} is independently selected from halogen and —C(O)NR^{q1}R^{r1}.

A specific value for each Rⁿ¹, R^{q1} and R^{r1} are each H.

A specific value for each Z^{1a} is independently selected from halogen, —OH and —C(O)NH₂.

A specific value for each Z^{1a} is independently selected from fluoro, —OH and —C(O)NH₂.

A specific value for R^{q1} and R^{r1} is H.

A specific value for Z¹ is selected from:

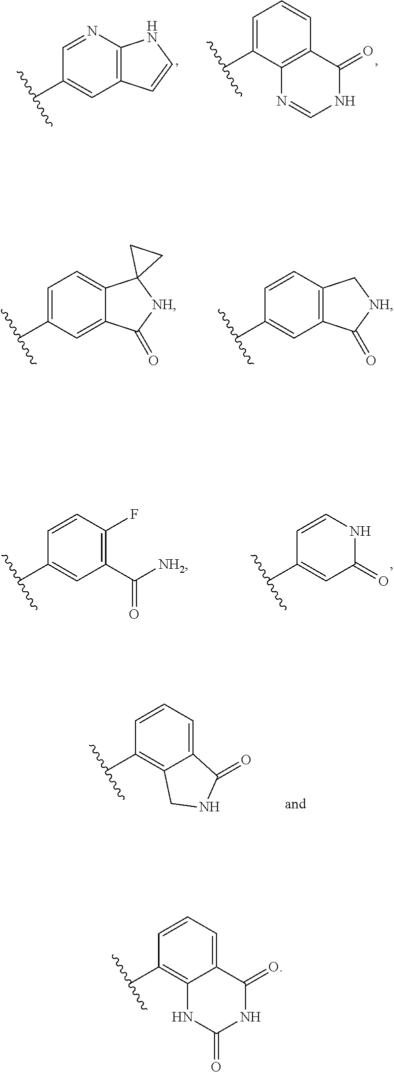

A specific value for Z² is selected from (C₂-C₈)alkynyl, 6-12 membered aryl, 5-12 membered C-linked-heteroaryl, 3-12 membered C-linked-heterocycle and —C(O)NR^{q3}R^{r3}, wherein any 6-12 membered aryl, 5-12 membered C-linked-heteroaryl and 3-12 membered C-linked-heterocycle of Z² is optionally substituted with one or more (e.g., 1, 2, 3, 4 or 5) Z^{2b} or Z^{2c} groups, and wherein any (C₂-C₈)alkynyl of Z² is optionally substituted with one or more (e.g., 1, 2, 3, 4 or 5) Z^{2c} groups.

A specific value for Z² is selected from (C₂-C₈)alkynyl, 6-12 membered aryl, 5-12 membered C-linked-heteroaryl, 3-12 membered C-linked-heterocycle and —C(O)NR^{q3}R^{r3}, wherein any 6-12 membered aryl, 5-12 membered C-linked-heteroaryl and 3-12 membered C-linked-heterocycle of Z² is optionally substituted with one or more (e.g., 1, 2, 3, 4 or 5) Z^{2c} groups, and wherein any (C₂-C₈)alkynyl of Z² is optionally substituted with one or more (e.g., 1, 2, 3, 4 or 5) Z^{2c} groups.

A specific value for Z² is selected from (C₂-C₈)alkynyl, phenyl, 5-6 membered C-linked-monocyclic-heteroaryl, 8-10 membered C-linked-bicyclic-heteroaryl, 8-10 membered C-linked-bicyclic-heterocycle and —C(O)NR^{q3}R^{r3}, wherein any phenyl, 5-6 membered C-linked-monocyclic-heteroaryl, 8-10 membered C-linked-bicyclic-heteroaryl and 8-10 membered C-linked-bicyclic-heterocycle of Z² is optionally substituted with one or more (e.g., 1, 2, 3, 4 or 5) Z^{2b} or Z^{2c} groups, and wherein any (C₂-C₈)alkynyl of Z² is optionally substituted with one or more (e.g., 1, 2, 3, 4 or 5) Z^{2c} groups.

A specific value for Z² is selected from (C₂-C₈)alkynyl, phenyl, 5-6 membered C-linked-monocyclic-heteroaryl, 8-10 membered C-linked-bicyclic-heteroaryl, 8-10 membered C-linked-bicyclic-heterocycle and —C(O)NR^{q3}R^{r3}, wherein any phenyl, 5-6 membered C-linked-monocyclic-heteroaryl, 8-10 membered C-linked-bicyclic-heteroaryl and 8-10 membered C-linked-bicyclic-heterocycle of Z² is optionally substituted with one or more (e.g., 1, 2, 3, 4 or 5) Z^{2c} groups, and wherein any (C₂-C₈)alkynyl of Z² is optionally substituted with one or more (e.g., 1, 2, 3, 4 or 5) Z^{2c} groups.

A specific value for Z² is selected from (C₂-C₈)alkynyl, phenyl, 5-6 membered C-linked-monocyclic-heteroaryl, 8-10 membered C-linked-bicyclic-heteroaryl, 8-10 membered C-linked-bicyclic-heterocycle and —C(O)NR^{q3}R^{r3}, wherein the 5-6 membered C-linked-monocyclic-heteroaryl, 8-10 membered C-linked-bicyclic-heteroaryl and 8-10 membered C-linked-bicyclic-heterocycle have 1-9 carbon atoms and 1-4 heteroatoms in the ring system, and wherein any phenyl, 5-6 membered C-linked-monocyclic-heteroaryl, 8-10 membered C-linked-bicyclic-heteroaryl, 8-10 membered and C-linked-bicyclic-heterocycle of Z² is optionally substituted with one or more (e.g., 1, 2, 3, 4 or 5) Z^{2b} or Z^{2c} groups, and wherein any (C₂-C₈)alkynyl of Z² is optionally substituted with one or more (e.g., 1, 2, 3, 4 or 5) Z^{2c} groups.

A specific value for Z² is selected from (C₂-C₈)alkynyl, phenyl, 5-6 membered C-linked-monocyclic-heteroaryl, 8-10 membered C-linked-bicyclic-heteroaryl, 8-10 membered C-linked-bicyclic-heterocycle and —C(O)NR^{q3}R^{r3}, wherein the 5-6 membered C-linked-monocyclic-heteroaryl, 8-10 membered C-linked-bicyclic-heteroaryl and 8-10 membered C-linked-bicyclic-heterocycle have 1-9 carbon atoms and 1-4 heteroatoms in the ring system, and wherein any phenyl, 5-6 membered C-linked-monocyclic-heteroaryl, 8-10 membered C-linked-bicyclic-heteroaryl, 8-10 membered and C-linked-bicyclic-heterocycle of Z² is optionally substituted with one or more (e.g., 1, 2, 3, 4 or 5) Z^{2c} groups, and wherein any (C₂-C₈)alkynyl of Z² is optionally substituted with one or more (e.g., 1, 2, 3, 4 or 5) Z^{2c} groups.

A specific value for Z² is selected from 4-methylpentynyl, phenyl, pyridinyl, 1H-2-oxo-pyridinyl, triazolyl, 1-oxoisoindolinyl, 1H-pyrrolo[2,3-b]pyridinyl and —C(O)NR^{q3}R^{r3}, wherein any phenyl, pyridinyl, 1H-2-oxo-pyridinyl, triazolyl, 1-oxoisoindolinyl and 1H-pyrrolo[2,3-b]pyridinyl of Z² is optionally substituted with one or more (e.g., 1, 2, 3, 4 or 5) Z^{2b} or Z^{2c} groups, and wherein any 4-methylpentynyl of Z² is optionally substituted with one or more (e.g., 1, 2, 3, 4 or 5) Z^{2c} groups.

A specific value for Z² is selected from 4-methylpentynyl, phenyl, pyridinyl, 1H-2-oxo-pyridinyl, triazolyl, 1-oxoisoindolinyl, 1H-pyrrolo[2,3-b]pyridinyl and —C(O)NR^{q3}R^{r3}, wherein any phenyl, pyridinyl, 2-oxopyridinyl, triazolyl, 1-oxoisoindolinyl and 1H-pyrrolo[2,3-b]pyridinyl of Z² is optionally substituted with one or more (e.g., 1, 2, 3, 4 or 5) Z^{2c} groups, and wherein any 4-methylpentynyl of Z² is optionally substituted with one or more (e.g., 1, 2, 3, 4 or 5) Z^{2c} groups.

A specific value for Z² is selected from 4-methylpentyn-1-yl, phenyl, pyridin-4-yl, 1H-2-oxo-pyridin-2-yl, triazol-4-yl, 1-oxoisoindolin-6-yl, 1H-pyrrolo[2,3-b]pyridine-5-yl and —C(O)NR^{q3}R^{r3}, wherein any phenyl, pyridin-4-yl, 1H-2-oxo-pyridin-2-yl, triazol-4-yl, 1-oxoisoindolin-6-yl and 1H-pyrrolo[2,3-b]pyridine-5-yl of Z² is optionally substituted with one or more (e.g., 1, 2, 3, 4 or 5) Z^{2b} or Z^{2c} groups, and wherein any 4-methylpentyn-1-yl of Z² is optionally substituted with one or more (e.g., 1, 2, 3, 4 or 5) Z^{2c} groups.

A specific value for Z² is selected from 4-methylpentyn-1-yl, phenyl, pyridin-4-yl, 1H-2-oxo-pyridin-2-yl, triazol-4-yl, 1-oxoisoindolin-6-yl, 1H-pyrrolo[2,3-b]pyridine-5-yl and —C(O)NR^{q3}R^{r3}, wherein any phenyl, pyridin-4-yl, 1H-2-oxo-pyridin-2-yl, triazol-4-yl, 1-oxoisoindolin-6-yl and 1H-pyrrolo[2,3-b]pyridine-5-yl of Z² is optionally substituted with one or more (e.g., 1, 2, 3, 4 or 5) Z^{2c} groups, and wherein any 4-methylpentyn-1-yl of Z² is optionally substituted with one or more (e.g., 1, 2, 3, 4 or 5) Z^{2c} groups.

A specific group of compounds of formula I are compounds wherein each Z² is not substituted with Z^{2b}.

A specific group of compounds of formula I are compounds wherein each Z² is optionally substituted with one or more Z^{2c} groups.

A specific value for each Z^{2c} is independently selected from halogen, —ORₙ₄ and —C(O)NR_{q4}Rᵣ₄.

A specific group of compounds of formula I are compounds wherein Rⁿ⁴ is H or methyl, and R^{q4} and R^{r4} are each H.

A specific value for Rⁿ⁴ is H or methyl.

A specific value for each R^{q4} and R^{r4} is H.

A specific value for Z² is selected from:

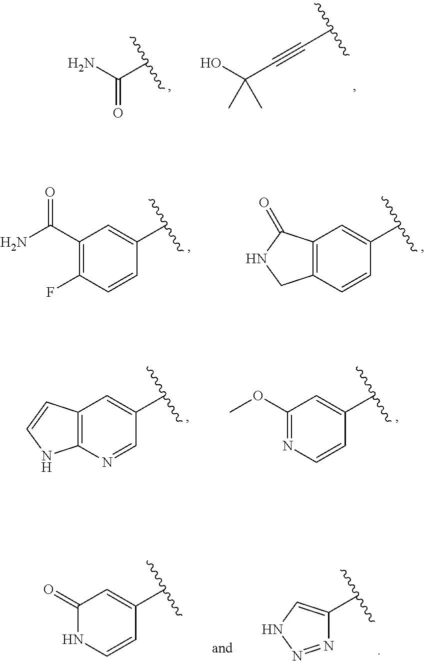

A specific value for A is selected from:

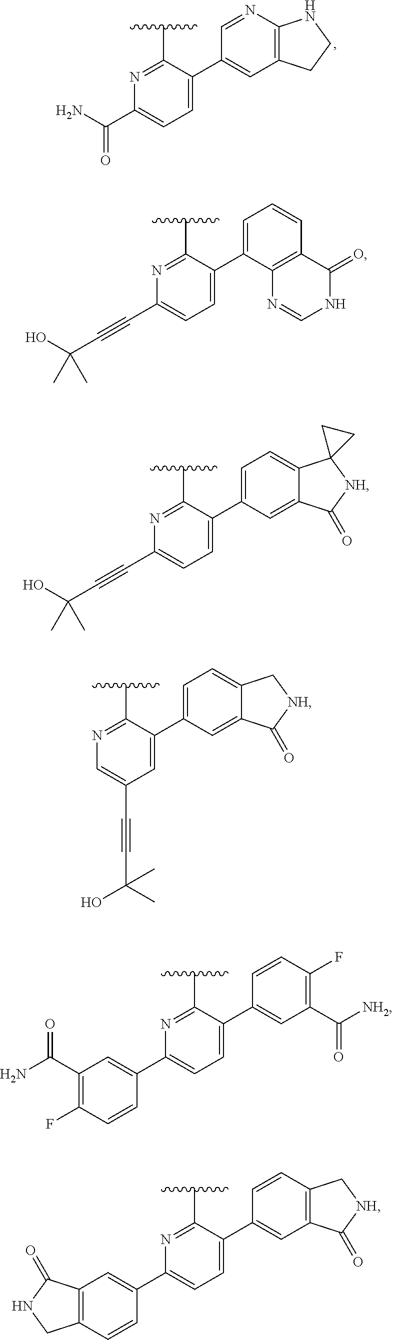 [SMILES: *c1nc(C(N)=O)ccc1-c1cnc2c(c1)CCN2 ; *c1nc(C#CC(C)(C)O)ccc1-c1cccc2c(=O)[nH]cnc12 ; *c1nc(C#CC(C)(C)O)ccc1-c1ccc2c(c1)C(=O)NC21CC1 ; *c1ncc(C#CC(C)(C)O)cc1-c1ccc2c(c1)C(=O)NC2 ; *c1nc(-c2ccc(F)c(C(N)=O)c2)ccc1-c1ccc(F)c(C(N)=O)c1 ; *c1nc(-c2ccc3c(c2)C(=O)NC3)ccc1-c1ccc2c(c1)C(=O)NC2]

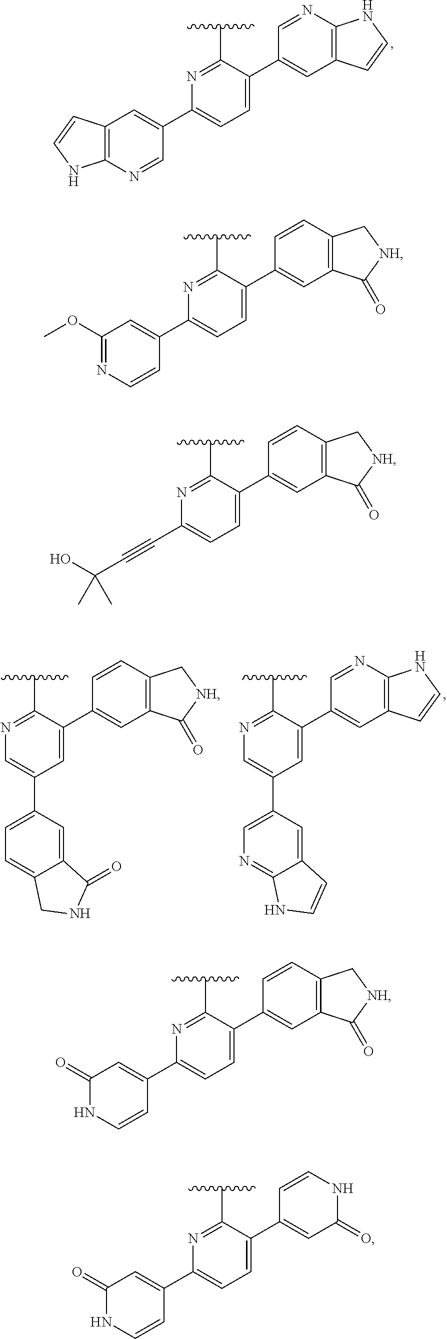

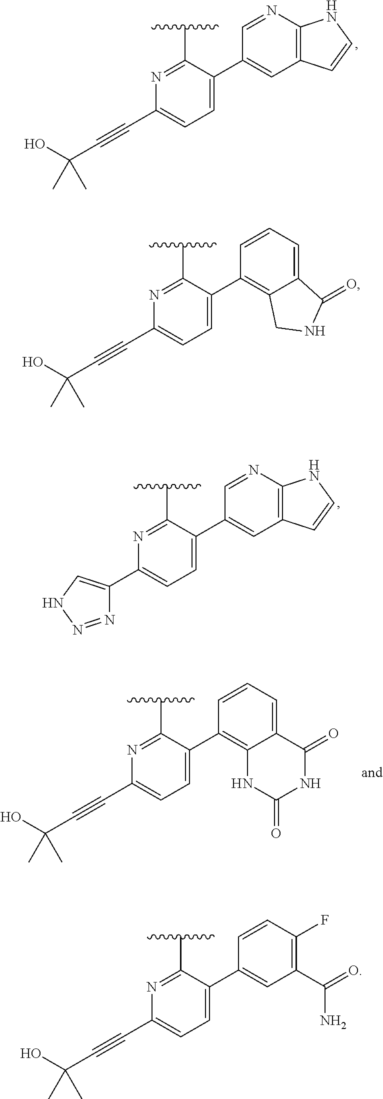 [SMILES: *c1nc(C#CC(C)(C)O)ccc1-c1cnc2[nH]ccc2c1 ; *c1nc(C#CC(C)(C)O)ccc1-c1cccc2c1CNC2=O ; *c1nc(-c2c[nH]nn2)ccc1-c1cnc2[nH]ccc2c1 ; *c1nc(C#CC(C)(C)O)ccc1-c1cccc2c(=O)[nH]c(=O)[nH]c12 ; *c1nc(C#CC(C)(C)O)ccc1-c1ccc(F)c(C(N)=O)c1]

A specific value for R¹ is a 5-12 membered heteroaryl, wherein any 5-12 membered heteroaryl of R¹ is optionally substituted with one or more (e.g., 1, 2, 3, 4, or 5) Z⁴ groups.

A specific value for R¹ is a 8-12 membered bicyclic-heteroaryl or 8-12 membered tricyclic-heteroaryl, wherein any 8-12 membered bicyclic-heteroaryl or 8-12 membered tricyclic-heteroaryl of R¹ is optionally substituted with one or more (e.g., 1, 2, 3, 4, or 5) Z⁴ groups.

A specific value for R¹ is a 8-12 membered bicyclic-heteroaryl or 8-12 membered tricyclic-heteroaryl, wherein the 8-12 membered bicyclic-heteroaryl or 8-12 membered tricyclic-heteroaryl have 4-10 carbon atoms and 1-5 heteroatoms in the ring system, and wherein any 8-12 membered bicyclic-heteroaryl or 8-12 membered tricyclic-heteroaryl of R¹ is optionally substituted with one or more (e.g., 1, 2, 3, 4, or 5) Z⁴ groups.

A specific value for R¹ is a 8-12 membered bicyclic-heteroaryl or 8-12 membered tricyclic-heteroaryl, wherein the 8-12 membered bicyclic-heteroaryl or 8-12 membered tricyclic-heteroaryl contains at least one partially unsaturated ring, and wherein any 8-12 membered bicyclic-heteroaryl or 8-12 membered tricyclic-heteroaryl of R¹ is optionally substituted with one or more (e.g., 1, 2, 3, 4 or 5) Z⁴ groups.

A specific value for R¹ has the following formula IIa:

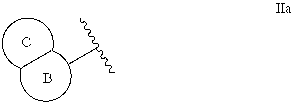

wherein:

C together with the two carbon atoms of ring B to which it is attached forms a 3-7 membered monocyclic-carbocycle, 5-8 membered bicyclic-carbocycle, 3-7 membered monocyclic-heterocycle or 5-8 membered bicyclic heterocycle, wherein any 3-7 membered monocyclic-carbocycle, 5-8 membered bicyclic-carbocycle, 3-7 membered monocyclic-heterocycle or 5-8 membered bicyclic heterocycle of C is optionally substituted with one or more (e.g. 1, 2, 3, 4 or 5) Z⁴ groups; and

B is a 5 or 6 membered monocyclic-heteroaryl with 1, 2 or 3 nitrogen atoms, wherein B is optionally substituted with one or more or (e.g. 1, 2, 3, 4 or 5) Z⁴ groups.

A specific value for R¹ has the following IIb:

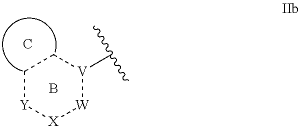

wherein:

C together with the two carbon atoms of ring B to which it is attached forms a 3-7 membered monocyclic-carbocycle, 5-8 membered bicyclic-carbocycle, 3-7 membered monocyclic-heterocycle or 5-8 membered bicyclic heterocycle, wherein any 3-7 membered monocyclic-carbocycle, 5-8 membered bicyclic-carbocycle, 3-7 membered monocyclic-heterocycle or 5-8 membered bicyclic heterocycle of C is optionally substituted with one or more (e.g. 1, 2, 3, 4 or 5) Z⁴ groups; and

B is a 5 or 6 membered monocyclic-heteroaryl having 1, 2 or 3 nitrogen atoms;

V is C or N;

W is CZ^{4c}, NZ^{4c} or N;

X is CZ^{4c}, NZ^{4c} or N;

Y is CZ^{4c}, N or absent;

the dashed bonds are selected from single bonds and double bonds, wherein the dashed bonds, V, W, X and Y are selected so that the 5 or 6 membered monocyclic-heteroaryl B is aromatic; and

each Z^{4c} is independently selected from H or Z⁴.

A specific value for R¹ has the following formula IIc:

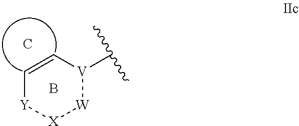

wherein:

C together with the two carbon atoms of ring B to which it is attached forms a 3-7 membered monocyclic-carbocycle, 5-8 membered bicyclic-carbocycle, 3-7 membered monocyclic-heterocycle or 5-8 membered bicyclic heterocycle, wherein any 3-7 membered monocyclic-carbocycle, 5-8 membered bicyclic-carbocycle, 3-7 membered monocyclic-heterocycle or 5-8 membered bicyclic heterocycle of C is optionally substituted with one or more (e.g. 1, 2, 3, 4 or 5) Z⁴ groups; and

B is a 5 or 6 membered monocyclic-heteroaryl having 1, 2 or 3 nitrogen atoms;

V is C or N;

W is CZ^{4c} or N;

X is CZ^{4c}, NZ^{4c} or N;

Y is CZ^{4c}, N or absent;

the dashed bonds are selected from single bonds and double bonds, wherein the dashed bonds, V, W, X and Y are selected so that the 5 or 6 membered monocyclic-heteroaryl B is aromatic; and

each Z^{4c} is independently selected from H or Z⁴.

A specific value for R¹ has the following R¹ has the following formula IId:

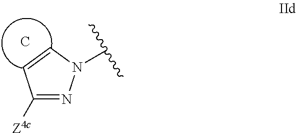

wherein:

C together with the two carbon atoms to which it is attached forms a 3-7 membered monocyclic-carbocycle, 5-9 membered bicyclic-carbocycle, 3-7 membered monocyclic-heterocycle or 5-9 membered bicyclic heterocycle, wherein any 3-7 membered monocyclic-carbocycle, 5-9 membered bicyclic-carbocycle, 3-7 membered monocyclic-heterocycle or 5-9 membered bicyclic heterocycle of C is optionally substituted with one or more (e.g. 1, 2, 3, 4 or 5) Z⁴ groups; and

each Z^{4c} is independently selected from H or Z⁴.

A specific value for each Z⁴ is independently selected from (C₁-C₆)alkyl and halogen, wherein any (C₁-C₆)alkyl of Z⁴ is optionally substituted with one or more (e.g., 1, 2, 3, 4, or 5) halogen.

A specific value for each Z⁴ is independently selected from fluoro, trifluoromethyl and difluoromethyl.

A specific value for R¹ is selected from:

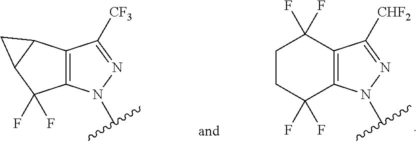 [SMILES: *n1nc(C(F)(F)F)c2c1C(F)(F)C1CC21 ; *n1nc(C(F)F)c2c1C(F)(F)CCC2(F)F]

A specific value for R¹ is selected from:

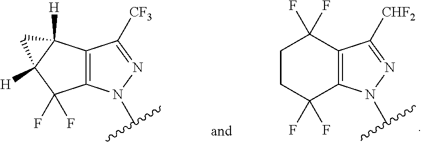 [SMILES: *n1nc(C(F)(F)F)c2c1C(F)(F)[C@@H]1C[C@H]21 ; *n1nc(C(F)F)c2c1C(F)(F)CCC2(F)F]

A specific value for R¹ is a 8-12 membered bicyclic-heteroaryl or 8-12 membered tricyclic-heteroaryl, wherein the 8-12 membered bicyclic-heteroaryl or 8-12 membered tricyclic-heteroaryl has 4-9 carbon atoms and 1-5 heteroatoms in the ring system, and wherein any 8-12 membered bicyclic-heteroaryl or 8-12 membered tricyclic-heteroaryl of R¹ is optionally substituted with one or more (e.g., 1, 2, 3, 4 or 5) Z⁴ groups.

A specific value for R¹ is a 8-12 membered bicyclic-heteroaryl, wherein the 8-12 membered bicyclic-heteroaryl has 6-9 carbon atoms and 1-3 heteroatoms in the ring system, and wherein any 8-12 membered bicyclic-heteroaryl of R¹ is optionally substituted with one or more (e.g., 1, 2, 3, 4 or 5) Z⁴ groups.

A specific value for R¹ is a 8-12 membered bicyclic-heteroaryl or 8-12 membered tricyclic-heteroaryl, wherein the 8-12 membered bicyclic-heteroaryl or 8-12 membered tricyclic-heteroaryl has 6-9 carbon atoms and 1-3 heteroatoms in the ring system, and wherein any 8-12 membered bicyclic-heteroaryl or 8-12 membered tricyclic-heteroaryl of R¹ is optionally substituted with one or more (e.g., 1, 2, 3, 4 or 5) Z⁴ groups.

A specific value for R¹ is selected from indolyl and 4,5,6,7-tetrahydro-indazolyl, wherein any indolyl and 4,5,6,7-tetrahydro-indazolyl of R¹ is optionally substituted with one or more (e.g., 1, 2, 3, 4 or 5) Z⁴ groups.

A specific value for R¹ is selected from indolyl, 4,5,6,7-tetrahydro-indazolyl, 3b,4,4a,5-tetrahydro-cyclopropa[3,4]cyclopenta[1,2-c]pyrazole and 1,4,5,5a,6,6a-hexahydrocyclopropa[g]indazole, wherein any indolyl, 4,5,6,7-tetrahydro-indazolyl, 3b,4,4a,5-tetrahydro-cyclopropa[3,4]cyclopenta[1,2-c]pyrazole and 1,4,5,5a,6,6a-hexahydrocyclopropa[g]indazole of R¹ is optionally substituted with one or more (e.g., 1, 2, 3, 4 or 5) Z⁴ groups.

A specific value for R¹ is selected from indol-3-yl and 4,5,6,7-tetrahydro-1H-indazol-1-yl, wherein any indol-3-yl and 4,5,6,7-tetrahydro-1H-indazol-1-yl of R¹ is optionally substituted with one or more (e.g., 1, 2, 3, 4 or 5) Z⁴ groups.

A specific value for R¹ is selected from indol-3-yl, 4,5,6,7-tetrahydro-1H-indazol-1-yl, 3b,4,4a,5-tetrahydro-1H-cyclopropa[3,4]cyclopenta[1,2-c]pyrazol-1-yl and 1,4,5,5a,6,6a-hexahydrocyclopropa[g]indazol-1-yl, wherein any indol-3-yl, 4,5,6,7-tetrahydro-1H-indazol-1-yl, 3b,4,4a,5-tetrahydro-1H-cyclopropa[3,4]cyclopenta[1,2-c]pyrazol-1-yl and 1,4,5,5a,6,6a-hexahydrocyclopropa[g]indazol-1-yl of R¹ is optionally substituted with one or more (e.g., 1, 2, 3, 4 or 5) Z⁴ groups.

A specific value for each Z⁴ is independently selected from (C₁-C₆)alkyl and halogen, wherein any (C₁-C₆)alkyl of Z⁴ is optionally substituted with one or more (e.g., 1, 2, 3, 4 or 5) halogen.

A specific value for each Z⁴ is independently selected from (C₁-C₆)alkyl, —CN and halogen, wherein any (C₁-C₆)alkyl of Z⁴ is optionally substituted with one or more (e.g., 1, 2, 3, 4 or 5) halogen.

A specific value for each Z⁴ is independently selected from fluoro, trifluoromethyl and difluoromethyl.

A specific value for each Z⁴ is independently selected from fluoro, trifluoromethyl, —CN and difluoromethyl.

A specific value for R¹ is selected from:

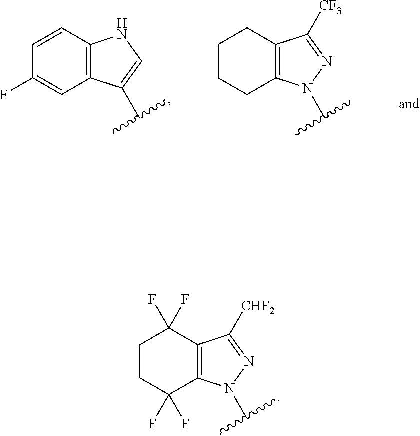 [SMILES: *c1c[nH]c2ccc(F)cc12 ; *n1nc(C(F)(F)F)c2c1CCCC2 ; *n1nc(C(F)F)c2c1C(F)(F)CCC2(F)F]

A specific value for R¹ is selected from:

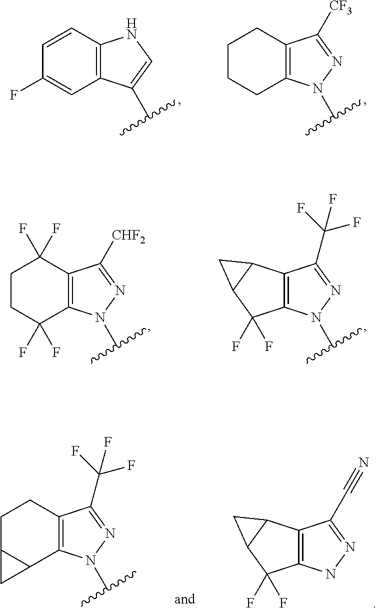 [SMILES: *c1c[nH]c2ccc(F)cc12 ; *n1nc(C(F)(F)F)c2c1CCCC2 ; *n1nc(C(F)F)c2c1C(F)(F)CCC2(F)F ; *n1nc(C(F)(F)F)c2c1C(F)(F)C1CC21 ; *n1nc(C(F)(F)F)c2c1C1CC1CC2 ; N#Cc1n[nH]c2c1C1CC1C2(F)F]

A specific value for R¹ is selected from:

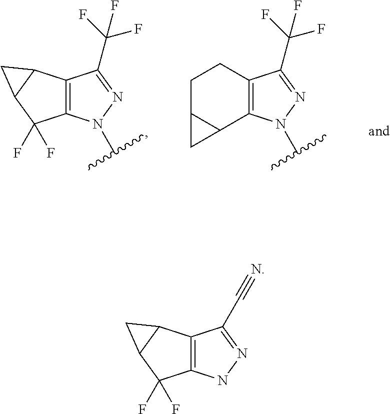 [SMILES: *n1nc(C(F)(F)F)c2c1C(F)(F)C1CC21 ; *n1nc(C(F)(F)F)c2c1C1CC1CC2 ; N#Cc1n[nH]c2c1C1CC1C2(F)F]

In one embodiment the compound of formula I is selected from:

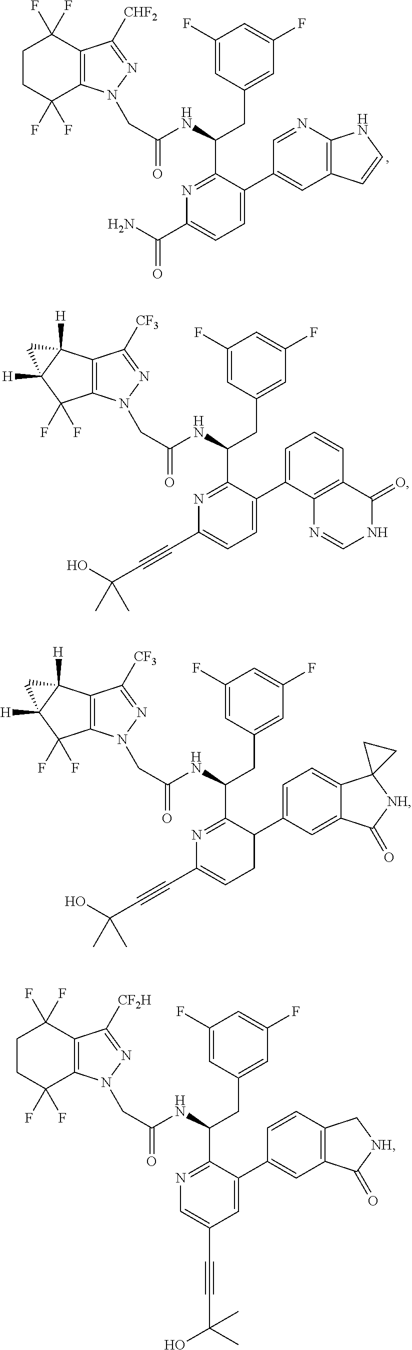

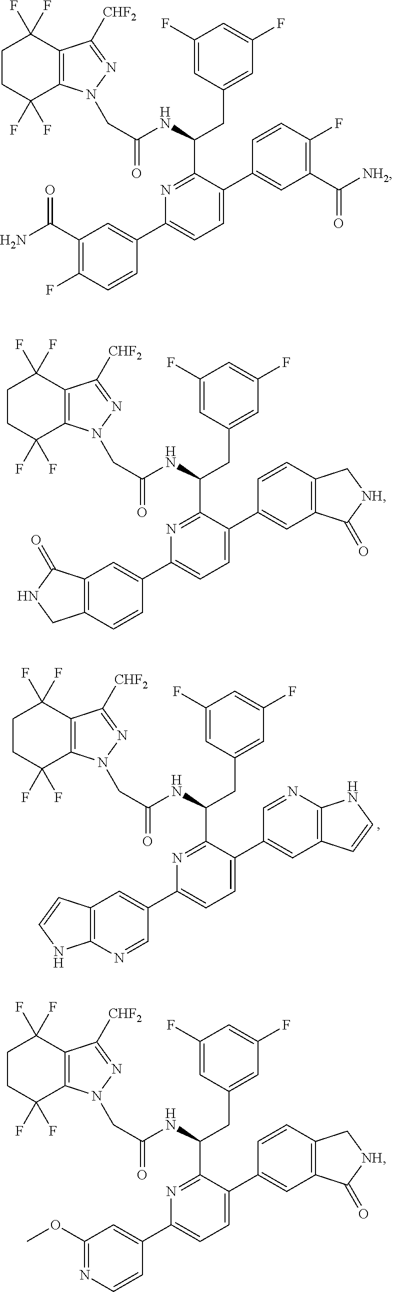 [SMILES: NC(=O)c1cc(-c2ccc(-c3ccc(F)c(C(N)=O)c3)c([C@H](Cc3cc(F)cc(F)c3)NC(=O)Cn3nc(C(F)F)c4c3C(F)(F)CCC4(F)F)n2)ccc1F ; O=C(Cn1nc(C(F)F)c2c1C(F)(F)CCC2(F)F)N[C@@H](Cc1cc(F)cc(F)c1)c1nc(-c2ccc3c(c2)C(=O)NC3)ccc1-c1ccc2c(c1)C(=O)NC2 ; O=C(Cn1nc(C(F)F)c2c1C(F)(F)CCC2(F)F)N[C@@H](Cc1cc(F)cc(F)c1)c1nc(-c2cnc3[nH]ccc3c2)ccc1-c1cnc2[nH]ccc2c1 ; COc1cc(-c2ccc(-c3ccc4c(c3)C(=O)NC4)c([C@H](Cc3cc(F)cc(F)c3)NC(=O)Cn3nc(C(F)F)c4c3C(F)(F)CCC4(F)F)n2)ccn1]

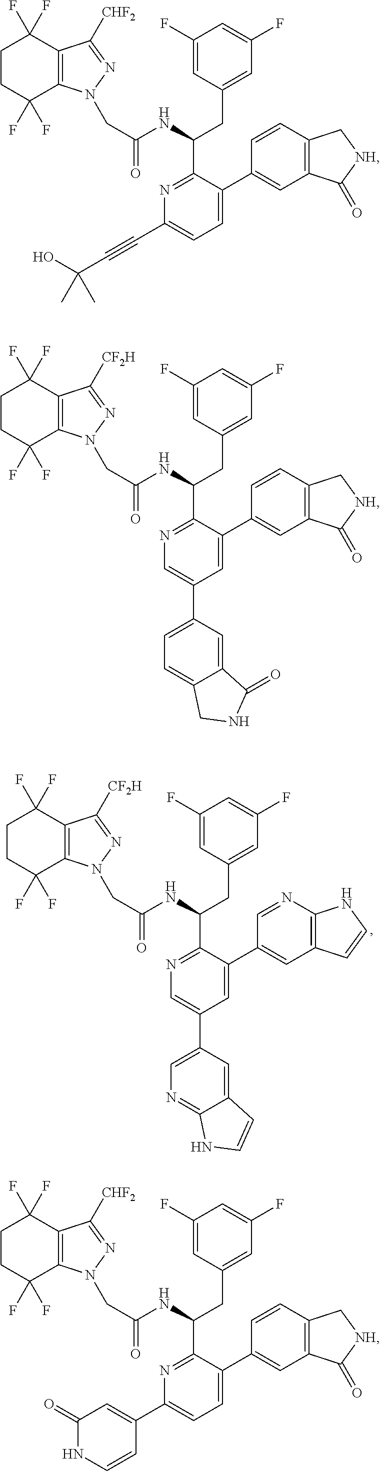 [SMILES: CC(C)(O)C#Cc1ccc(-c2ccc3c(c2)C(=O)NC3)c([C@H](Cc2cc(F)cc(F)c2)NC(=O)Cn2nc(C(F)F)c3c2C(F)(F)CCC3(F)F)n1 ; O=C(Cn1nc(C(F)F)c2c1C(F)(F)CCC2(F)F)N[C@@H](Cc1cc(F)cc(F)c1)c1ncc(-c2ccc3c(c2)C(=O)NC3)cc1-c1ccc2c(c1)C(=O)NC2 ; O=C(Cn1nc(C(F)F)c2c1C(F)(F)CCC2(F)F)N[C@@H](Cc1cc(F)cc(F)c1)c1ncc(-c2cnc3[nH]ccc3c2)cc1-c1cnc2[nH]ccc2c1 ; O=C(Cn1nc(C(F)F)c2c1C(F)(F)CCC2(F)F)N[C@@H](Cc1cc(F)cc(F)c1)c1nc(-c2cc[nH]c(=O)c2)ccc1-c1ccc2c(c1)C(=O)NC2]

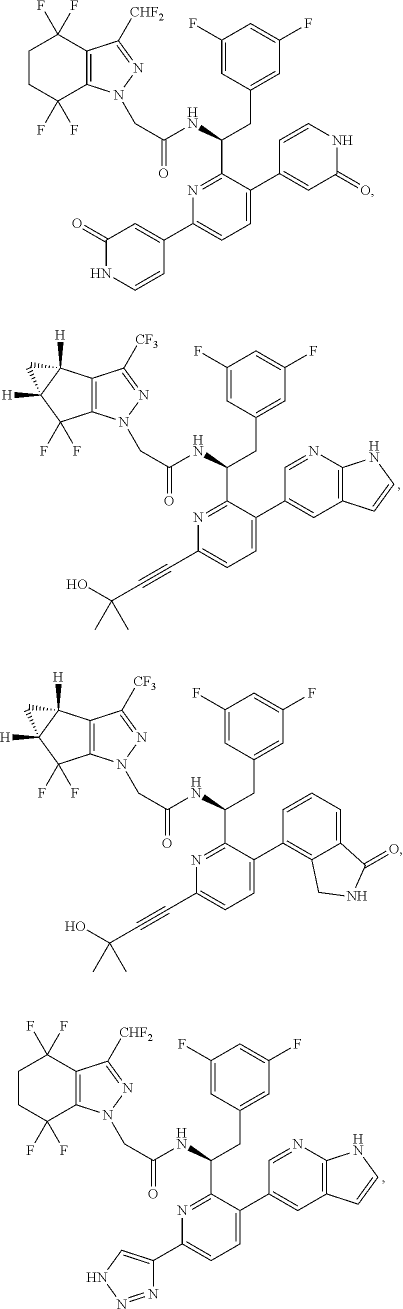 [SMILES: O=C(Cn1nc(C(F)F)c2c1C(F)(F)CCC2(F)F)N[C@@H](Cc1cc(F)cc(F)c1)c1nc(-c2cc[nH]c(=O)c2)ccc1-c1cc[nH]c(=O)c1 ; CC(C)(O)C#Cc1ccc(-c2cnc3[nH]ccc3c2)c([C@H](Cc2cc(F)cc(F)c2)NC(=O)Cn2nc(C(F)(F)F)c3c2C(F)(F)[C@@H]2C[C@H]32)n1 ; CC(C)(O)C#Cc1ccc(-c2cccc3c2CNC3=O)c([C@H](Cc2cc(F)cc(F)c2)NC(=O)Cn2nc(C(F)(F)F)c3c2C(F)(F)[C@@H]2C[C@H]32)n1 ; O=C(Cn1nc(C(F)F)c2c1C(F)(F)CCC2(F)F)N[C@@H](Cc1cc(F)cc(F)c1)c1nc(-c2c[nH]nn2)ccc1-c1cnc2[nH]ccc2c1]

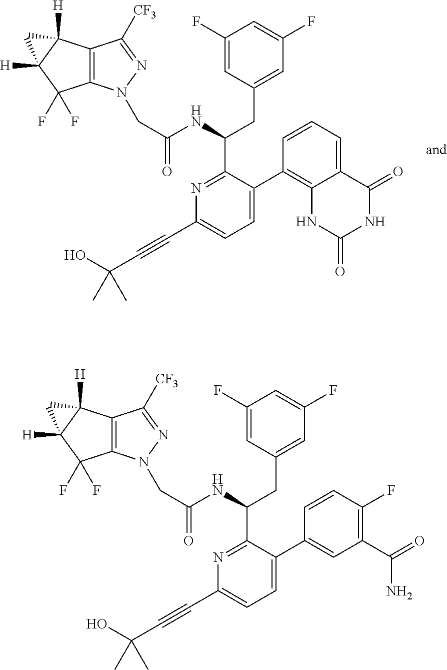 [SMILES: CC(C)(O)C#Cc1ccc(-c2cccc3c(=O)[nH]c(=O)[nH]c23)c([C@H](Cc2cc(F)cc(F)c2)NC(=O)Cn2nc(C(F)(F)F)c3c2C(F)(F)[C@@H]2C[C@H]32)n1 ; CC(C)(O)C#Cc1ccc(-c2ccc(F)c(C(N)=O)c2)c([C@H](Cc2cc(F)cc(F)c2)NC(=O)Cn2nc(C(F)(F)F)c3c2C(F)(F)[C@@H]2C[C@H]32)n1]

and pharmaceutically acceptable salts thereof.

### General Synthetic Procedures

The following scheme describes methods that are useful for preparing compounds of formula I.

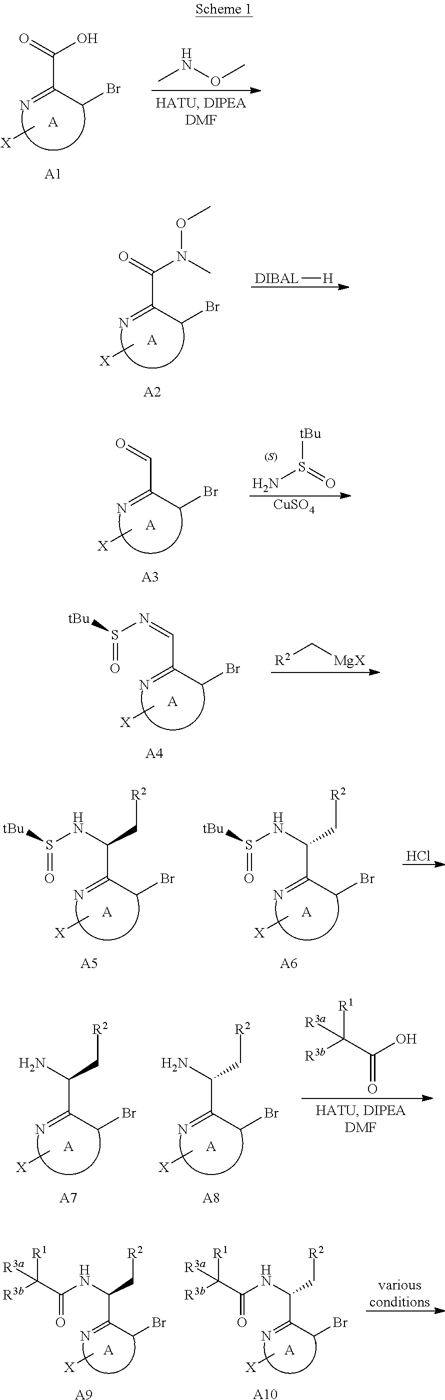

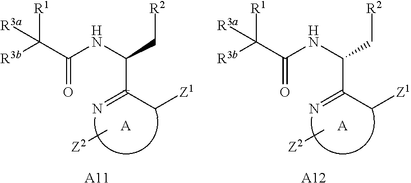

Scheme 1 describes a general stereoselective route which can be used to prepare compounds of formula I. Heteroaryl acids of formula A1 (where X represents diversifiable chemical group such as NH₂, SH, or halogen that may be suitably protected) can be converted to the corresponding aldehydes then condensed with a chiral auxiliary to provide a stereoselective addition of a nucleophilic reagent. Depicted in Scheme 1 is the conversion of a heteroaryl acid A1 containing two diversifiable functional groups (e.g., X and Br) to the corresponding aldehyde. This is followed by the condensation of the aldehyde A3 with (S) tert-butane sulfonamide and the addition of a Grignard reagent to provide a mixture of A5 and A6 enriched in A5. This mixture may be separated by column chromatography on silica gel to provide pure diastereomers. Removal of the auxiliary provides amines A7 and A8 which can be coupled to a variety of carboxylic acids to provide heteroaryl compounds of formula A9 and A10. Diversification of A9 and A10 may be accomplished by a variety of methods including alkylation, acylation, cyanation, nucleophilic aromatic displacement, and metal catalyzed cross coupling reactions such as Suzuki couplings, Buchwald-Hartwig type couplings, and Sonogashira couplings.

### Combination Therapy

In one embodiment, a method for treating an HIV infection is provided, comprising administering to a patient in need thereof a therapeutically effective amount of a compound disclosed herein, or a pharmaceutically acceptable salt, thereof, in combination with a therapeutically effective amount of one or more additional therapeutic agents which are suitable for treating an HIV infection.

In one embodiment, pharmaceutical compositions comprising a compound disclosed herein, or a pharmaceutically acceptable salt thereof, in combination with at least one additional therapeutic agent, and a pharmaceutically acceptable carrier are provided. For example, the therapeutic agent used in combination with the compound disclosed herein can be any anti-HIV agent.

One embodiment provides pharmaceutical compositions comprising a compound disclosed herein, or a pharmaceutically acceptable salt thereof, in combination with at least one additional therapeutic agent selected from the group consisting of HIV protease inhibiting compounds, HIV non-nucleoside inhibitors of reverse transcriptase, HIV nucleoside inhibitors of reverse transcriptase, HIV nucleotide inhibitors of reverse transcriptase, HIV integrase inhibitors, gp41 inhibitors, CXCR4 inhibitors, gp120 inhibitors, CCR5 inhibitors, capsid polymerization inhibitors, and other drug for treating HIV, and combinations thereof, and a pharmaceutically acceptable carrier.

One embodiment provides pharmaceutical compositions comprising a compound disclosed herein, or a pharmaceutically acceptable salt thereof, in combination with at least one additional therapeutic agent selected from the group consisting of:

(1) HIV protease inhibiting compounds selected from the group consisting of amprenavir, atazanavir, fosamprenavir, indinavir, lopinavir, ritonavir, nelfinavir, saquinavir, tipranavir, brecanavir, darunavir, TMC-126, TMC-114, mozenavir (DMP-450), JE-2147 (AG1776), L-756423, RO0334649, KNI-272, DPC-681, DPC-684, GW640385X, DG17, PPL-100, DG35, and AG 1859;

(2) HIV non-nucleoside inhibitors of reverse transcriptase selected from the group consisting of capravirine, emivirine, delaviridine, efavirenz, nevirapine, (+) calanolide A, etravirine, GW5634, DPC-083, DPC-961, DPC-963, MIV-150, and TMC-120, rilpivirene, BILR 355 BS, VRX 840773, UK-453061, RDEA806 and KM023;

(3) HIV nucleoside inhibitors of reverse transcriptase selected from the group consisting of zidovudine, emtricitabine, didanosine, stavudine, zalcitabine, lamivudine, abacavir, amdoxovir, elvucitabine, alovudine, MIV-210, ±-FTC, D-d4FC, emtricitabine, phosphazide, fozivudine tidoxil, apricitibine (AVX754), amdoxovir, KP-1461, and fosalvudine tidoxil (formerly HDP 99.0003);

(4) HIV nucleotide inhibitors of reverse transcriptase selected from the group consisting of tenofovir, tenofovir disoproxil fumarate, tenofovir alafenamide fumarate (Gilead Sciences), adefovir, adefovir dipivoxil, CMX-001 (Chimerix) or CMX-157 (Chimerix);

(5) HIV integrase inhibitors selected from the group consisting of curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, S-1360, AR-177, L-870812, and L-870810, raltegravir, BMS-538158, GSK364735C, BMS-707035, MK-2048, BA 011, GS-5696, elvitegravir and dolutegravir;

(6) gp41 inhibitors selected from the group consisting of enfuvirtide, sifuvirtide, FB006M, and TRI-1144;

(7) the CXCR4 inhibitor AMD-070;

(8) the entry inhibitor SP01A;

(9) the gp120 inhibitor BMS-488043;

(10) the G6PD and NADH-oxidase inhibitor immunitin;

(11) CCR5 inhibitors selected from the group consisting of aplaviroc, vicriviroc, maraviroc, PRO-140, INCB15050, PF-232798 (Pfizer), and CCR5mAb004;

(12) other drugs for treating HIV selected from the group consisting of BAS-100, SPI-452, REP 9, SP-01A, TNX-355, DES6, ODN-93, ODN-112, VGV-1, PA-457 (bevirimat), HRG214, VGX-410, KD-247, AMZ 0026, CYT 99007A-221 HIV, DEBIO-025, BAY 50-4798, MDX010 (ipilimumab), PBS119, ALG 889, and PA-1050040 (PA-040).

In some embodiments, one or more of the compounds disclosed herein are combined with one or more other active therapeutic agents in a unitary dosage form for simultaneous or sequential administration to a patient. The combination therapy may be administered as a simultaneous or sequential regimen. When administered sequentially, the combination may be administered in two or more administrations.

In some embodiments, one or more of the compounds disclosed herein are co-administered with one or more other active therapeutic agents. Co-administration of a compound disclosed herein with one or more other active therapeutic agents generally refers to simultaneous or sequential administration of a compound disclosed herein and one or more other active therapeutic agents, such that therapeutically effective amounts of disclosed herein and one or more other active therapeutic agents are both present in the body of the patient.

In yet another embodiment, a method for treating an HIV infection is provided, which method comprises administering to a patient in need thereof a therapeutically effective amount of a compound disclosed herein, or a pharmaceutically acceptable salt thereof, in combination with a therapeutically effective amount of one or more additional therapeutic agents such as those disclosed above.

### Pharmaceutical Formulations

The compounds disclosed herein are formulated with conventional carriers (e.g., inactive ingredient or excipient material) which will be selected in accord with ordinary practice. Tablets will contain excipients including glidants, fillers, binders and the like. Aqueous formulations are prepared in sterile form, and when intended for delivery by other than oral administration generally will be isotonic. All formulations will optionally contain excipients such as those set forth in the Handbook of Pharmaceutical Excipients (1986). Excipients include ascorbic acid and other antioxidants, chelating agents such as EDTA, carbohydrates such as dextrin, hydroxyalkylcellulose, hydroxyalkylmethylcellulose, stearic acid and the like. One embodiment provides the formulation as a solid dosage form including a solid oral dosage form. The pH of the formulations ranges from about 3 to about 11, but is ordinarily about 7 to 10.

While it is possible for the active ingredients to be administered alone it may be preferable to present them as pharmaceutical formulations (compositions). The formulations, both for veterinary and for human use, of the invention comprise at least one active ingredient, as above defined, together with one or more acceptable carriers and optionally other therapeutic ingredients. The carrier(s) must be “acceptable” in the sense of being compatible with the other ingredients of the formulation and physiologically innocuous to the recipient thereof.

The formulations include those suitable for the foregoing administration routes. The formulations may conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with inactive ingredients (e.g., a carrier, pharmaceutical excipients, etc.) which constitutes one or more accessory ingredients. In general the formulations are prepared by uniformly and intimately bringing into association the active ingredient with liquid carriers or finely divided solid carriers or both, and then, if necessary, shaping the product.

Formulations described herein that are suitable for oral administration may be presented as discrete units including but not limited to capsules, cachets or tablets each containing a predetermined amount of the active ingredient.

Pharmaceutical formulations disclosed herein comprise one or more compounds disclosed herein together with one or more pharmaceutically acceptable carriers or excipients and optionally other therapeutic agents. Pharmaceutical formulations containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, lactose monohydrate, croscarmellose sodium, povidone, calcium or sodium phosphate; granulating and disintegrating agents, such as maize starch, or alginic acid; binding agents, such as cellulose, microcrystalline cellulose, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

The amount of active ingredient that is combined with the inactive ingredients to produce a dosage form will vary depending upon the host treated and the particular mode of administration. For example, in some embodiments, a dosage form for oral administration to humans contains approximately 1 to 1000 mg of active material formulated with an appropriate and convenient amount of carrier material (e.g., inactive ingredient or excipient material). In certain embodiments, the carrier material varies from about 5 to about 95% of the total compositions (weight:weight).

It should be understood that in addition to the ingredients particularly mentioned above the formulations of this invention may include other agents conventional in the art having regard to the type of formulation in question, for example those suitable for oral administration may include flavoring agents.

The invention further provides veterinary compositions comprising at least one active ingredient as above defined together with a veterinary carrier.

Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

Effective dose of active ingredient depends at least on the nature of the condition being treated, toxicity, whether the compound is being used prophylactically (lower doses), the method of delivery, and the pharmaceutical formulation, and will be determined by the clinician using conventional dose escalation studies.

### Routes of Administration

One or more compounds disclosed herein (herein referred to as the active ingredients) are administered by any route appropriate to the condition to be treated. Suitable routes include oral, rectal, nasal, topical (including buccal and sublingual), vaginal and parenteral (including subcutaneous, intramuscular, intravenous, intradermal, intrathecal and epidural), and the like. It will be appreciated that the preferred route may vary with for example the condition of the recipient. An advantage of the compounds disclosed herein is that they are orally bioavailable and can be dosed orally.

The antiviral properties of a compound of the invention may be determined using Test A described below.

### Test A: Antiviral Assay in MT4 Cells

For the antiviral assay, 40 μL of 1× test concentration of 3-fold serially diluted compound in culture medium with 10% FBS was added to each well of a 384-well plate (10 concentrations) in quadruplicate. MT-4 cells were next mixed with HIV-IIIb at an m.o.i of 0.003 for 1 hour, after which time 35 μL of virus/cell mixture (2000 cells) was immediately added to each well containing 40 μL of diluted compound. The plates were then incubated at 37° C. for 5 days. After 5 days of incubation, 25 μl of 2× concentrated CellTiter-Glo™ Reagent (catalog #G7571, Promega Biosciences, Inc., Madison, Wis.) was added to each well containing MT-4 cells. Cell lysis was carried out by incubating at room temperature for 10 min and then chemiluminescence was read. EC50 values were defined as the compound concentration that caused a 50% decrease in luminescence signal, a measure of HIV-1 replication. Percent inhibition of virus-induced cell killing calculated from the dose response curve at 2 μM drug concentration is shown in the table below.

### Test B: Cytotoxicity Assay

Compound cytotoxicity and the corresponding CC50 values was determined using the same protocol as described in the antiviral assay (Test A) except that uninfected cells were used.

Compounds of the present invention demonstrate antiviral activity (Test A) as depicted in the table below. Shown below are the corresponding values for CC50 and percent inhibition of virus-induced cell killing in the presence of 2 μM drug concentration.

| Compound | % inhibition at 2 μM | CC50 (nM) |
|---|---|---|
| 1B | 77 | 8569 |
| 2 | 90 | 14347 |
| 3D | 82 | 4149 |
| 4H | 74 | 22793 |
| 5G | 58 | >53192 |
| 6 | 73 | >53192 |
| 7 | 92 | 5664 |
| 8C | 86 | 21955 |
| 9B | 95 | 14557 |
| 10B | 85 | >53192 |
| 11 | 66 | >53192 |
| 12 | 58 | >53192 |
| 13 | 0 | >53192 |
| 14E | 89 | 6824 |
| 15 | 94 | 10261 |
| 16C | 65 | 3670 |
| 17 | 80 | 12556 |
| 18 | 90 | 6934 |

In one embodiment, the compounds demonstrate >10% inhibition at 2 μM. In one embodiment, the compounds demonstrate >30% inhibition at 2 μM. In one embodiment, the compounds demonstrate >50% inhibition at 2 μM. In one embodiment, the compounds demonstrate >70% inhibition at 2 μM. It is to be understood that the compounds disclosed herein can be grouped according to their % inhibition as described above.

The specific pharmacological responses observed may vary according to and depending on the particular active compound selected or whether there are present pharmaceutical carriers, as well as the type of formulation and mode of administration employed, and such expected variations or differences in the results are contemplated in accordance with practice of the present invention.

The Examples provided herein describe the synthesis of compounds disclosed herein as well as intermediates used to prepare the compounds. It is to be understood that individual steps described herein may be combined. It is also to be understood that separate batches of a compound may be combined and then carried forth in the next synthetic step.

### Example 1

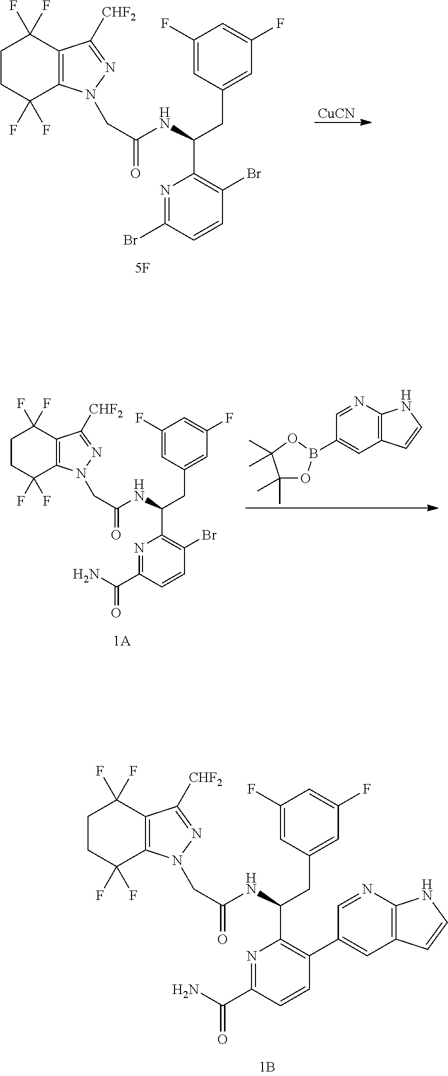

### Synthesis of (S)-5-bromo-6-(1-(2-(3-(difluoromethyl)-4,4,7,7-tetrafluoro-4,5,6,7-tetrahydro-1H-indazol-1-yl)acetamido)-2-(3,5-difluorophenyl)ethyl)picolinamide (1A)

Compound 5F (100 mg, 0.15 mmol) and CuCN (16 mg, 0.18 mmol) was dissolved in 0.3 mL of DMF. The reaction mixture was heated at 100° C. overnight. After cooled down to room temperature it was diluted with water and extracted with EtOAc. The organic phase was dried (Na₂SO₄), filtered and concentrated. The crude material was purified on reverse phase HPLC eluting with acetonitrile and water (with 0.1% TFA) to afford (S)—N-(1-(3-bromo-6-cyanopyridin-2-yl)-2-(3,5-difluorophenyl)ethyl)-2-(3-(difluoromethyl)-4,4,7,7-tetrafluoro-4,5,6,7-tetrahydro-1H-indazol-1-yl)acetamide and the title product (1A). MS (m/z) 640.05 [M+H]⁺.

### Synthesis of (S)-6-(1-(2-(3-(difluoromethyl)-4,4,7,7-tetrafluoro-4,5,6,7-tetrahydro-1H-indazol-1-yl)acetamido)-2-(3,5-difluorophenyl)ethyl)-5-(1H-pyrrolo[2,3-b]pyridin-5-yl)picolinamide (1B)

The title compound (1B) was prepared according to the method presented for the synthesis of compound 4H of Example 4 utilizing 5-(4,4,5,5-tetramethyl-1,3,2-dioxaborolan-2-yl)-1H-pyrrolo[2,3-b]pyridine and 1A. ¹H NMR (400 MHz, CD₃OD) δ 9.00 (d, J=8.5 Hz, 1H), 8.12 (d, J=7.9 Hz, 1H), 8.01 (s, 1H), 7.92-7.77 (m, 2H), 7.56 (d, J=3.5 Hz, 1H), 6.97-6.53 (m, 3H), 6.26 (d, J=6.1 Hz, 2H), 5.53 (m, 1H), 5.11 (s, 2H), 3.07 (m, 2H), 2.63-2.25 (m, 4H). MS (m/z) 678.08 [M+H]⁺.

### Example 2

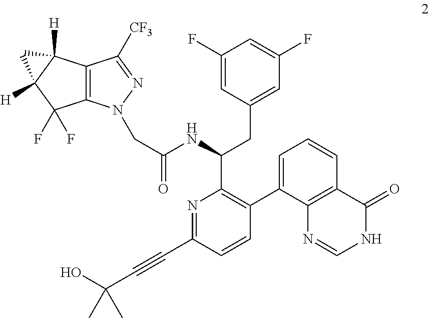 [SMILES: CC(C)(O)C#Cc1ccc(-c2cccc3c(=O)[nH]cnc23)c([C@H](Cc2cc(F)cc(F)c2)NC(=O)Cn2nc(C(F)(F)F)c3c2C(F)(F)[C@@H]2C[C@H]32)n1]

### Synthesis of 2-((3bS,4aR)-5,5-difluoro-3-(trifluoromethyl)-3b,4,4a,5-tetrahydro-1H-cyclopropa[3,4]cyclopenta[1,2-c]pyrazol-1-yl)-N—((S)-2-(3,5-difluorophenyl)-1-(6-(3-hydroxy-3-methylbut-1-yn-1-yl)-3-(4-oxo-3,4-dihydroquinazolin-8-yl)pyridin-2-yl)ethyl)acetamide (2)

The title compound (2) was prepared according to the method presented for the synthesis of compound 4H of Example 4 utilizing (4-oxo-3,4-dihydroquinazolin-8-yl)boronic acid and 14D. ¹H NMR (400 MHz, CD₃OD) δ 8.27 (m, 1H), 7.82 (m, 1H), 7.75 (m, 1H), 7.50 (s, 1H), 7.44 (m, 2H), 6.86 (m, 1H), 6.61 (m, 2H), 6.32 (m, 1H), 6.15 (m, 2H), 5.21 (m, 1H), 4.76 (s, 2H), 3.11 (m, 2H), 2.92 (m, 2H), 2.48 (m, 4H), 1.62 (d, J=6.6 Hz, 6H), 1.33 (m, 1H), 1.12 (m, 1H). MS (m/z) 725.14 [M+H]⁺.

### Example 3

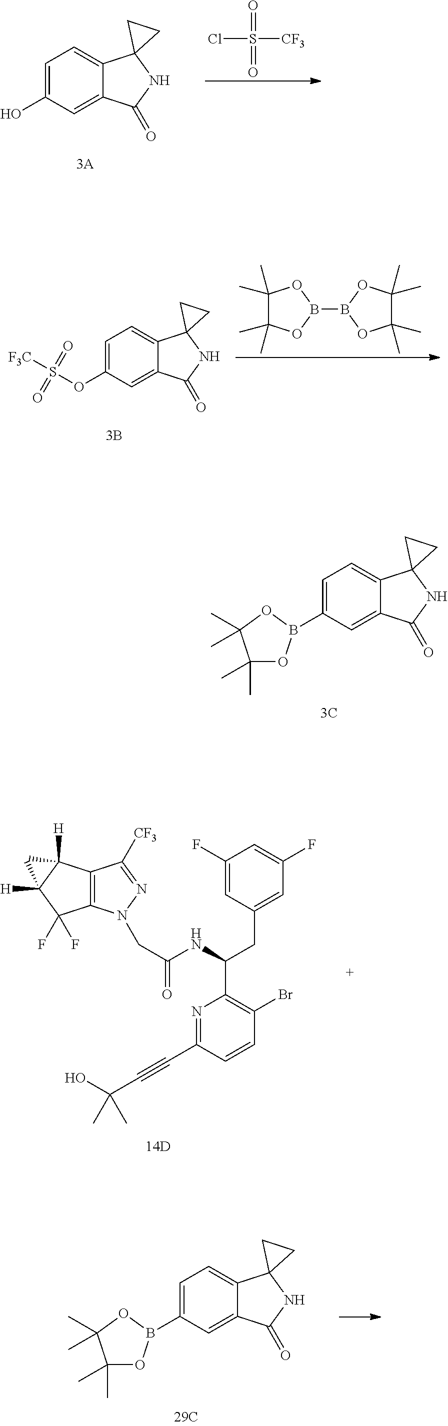

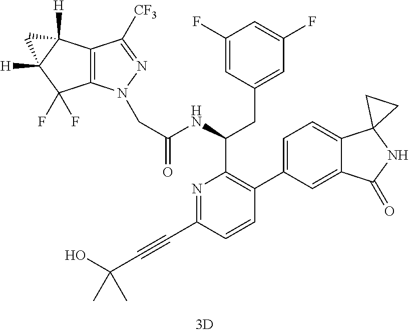 [SMILES: CC(C)(O)C#Cc1ccc(-c2ccc3c(c2)C(=O)NC32CC2)c([C@H](Cc2cc(F)cc(F)c2)NC(=O)Cn2nc(C(F)(F)F)c3c2C(F)(F)[C@@H]2C[C@H]32)n1]

### Synthesis of 3′-oxospiro[cyclopropane-1,1′-isoindolin]-5′-yl trifluoromethanesulfonate (3B)

The mixture of compound 3A (1 g, 5.7 mmol, prepared according to the method presented in Tetrahedron Letters 50 (2009) 1267-1269), DCM (20 mL), and Et₃N (0.9 mL, 6.8 mmol) was cooled to 0° C. using an ice/water bath. Trifluoromethanesulfonyl chloride (0.91 mL, 8.5 mmol) was added dropwise via syringe. The mixture was then stirred for 1 h in ambient temperature. More Trifluoromethanesulfonyl chloride (0.8 mL) was added and the mixture was stirred at ambient temperature for another hour. Then diluted with DCM (150 mL) and washed with 1.0 N HCl (50 mL), saturated aqueous sodium bicarbonate (1×50 mL), and saturated aqueous sodium chloride (1×50 mL). The organic layer was dried over MgSO₄, filtered through Celite^{.(R).}, and concentrated in vacuo to give the title product (3B). MS (m/z) 308.29 [M+H]⁺.

### Synthesis of 5′-(4,4,5,5-tetramethyl-1,3,2-dioxaborolan-2-yl)spiro[cyclopropane-1,1′-isoindolin]-3′-one (3C)

In a microwave tube were charged 3B (200 mg, 0.65 mmol), bis(pinacolato)diboron (330 mg, 1.3 mmol) and potassium acetate (191 mg, 1.95 mmol), [1,1′-Bis(diphenylphosphino)ferrocene]dichloropalladium(II)(14 mg, 0.02 mmol) and 1,4-dioxane (8 mL) The mixture was heated up to 150° C. for 20 min in a Microwave Synthesizer. Upon completion the solution was diluted in EtOAc and the organic layer was washed with water and a saturated NaCl solution, dried over MgSO₄ and concentrated in vacuum to give the title compound as a dark brown solid. A half amount of the product was purified by silica gel chromatography eluting with EtOAc/hexanes to afford the title product. MS (m/z) 286.23 [M+H]⁺.

### Synthesis of 2-((3bS,4aR)-5,5-difluoro-3-(trifluoromethyl)-3b,4,4a,5-tetrahydro-1H-cyclopropa[3,4]cyclopenta[1,2-c]pyrazol-1-yl)-N—((S)-2-(3,5-difluorophenyl)-1-(6-(3-hydroxy-3-methylbut-1-yn-1-yl)-3-(3′-oxospiro[cyclopropane-1,1′-isoindolin]-5′-yl)pyridin-2-yl)ethyl)acetamide (3D)

In a microwave tube were charged with 14D (33 mg, 0.05 mmol), 3C (21 mg, 0.075 mmol), LiCl (6 mg, 0.15 mmol), K₂CO₃ (21 mg, 0.15 mmol), Pd(PPh₃)₂Cl₂ (3 mg) and Pd(dppf)Cl₂ (3 mg). To the mixture was added 1 mL of DME and 0.2 mL of H₂O. The mixture was heated up to 165° C. for 12 min in a Microwave Synthesizer. After cooled down and filtered through a syringe filter, purified on reverse phase HPLC eluting with acetonitrile and water (with 0.1% TFA) to afford the title product. ¹H NMR (400 MHz, CD₃OD) δ 8.77 (d, J=8.4 Hz, 1H), 7.55 (d, J=8.0 Hz, 1H), 7.46 (d, J=8.0 Hz, 1H), 7.40 (d, J=7.8 Hz, 1H), 7.32-7.16 (m, 2H), 6.64 (t, J=9.2 Hz, 1H), 6.24 (d, J=6.5 Hz, 2H), 5.39 (t, J=7.3 Hz, 1H), 4.86 (s, 2H), 3.08-2.92 (m, 2H), 2.58-2.31 (m, 2H), 1.62 (s, 6H), 1.60-1.33 (m, 5H), 1.12 (m, 1H). MS (m/z) 738.15 [M+H]⁺.

### Example 4

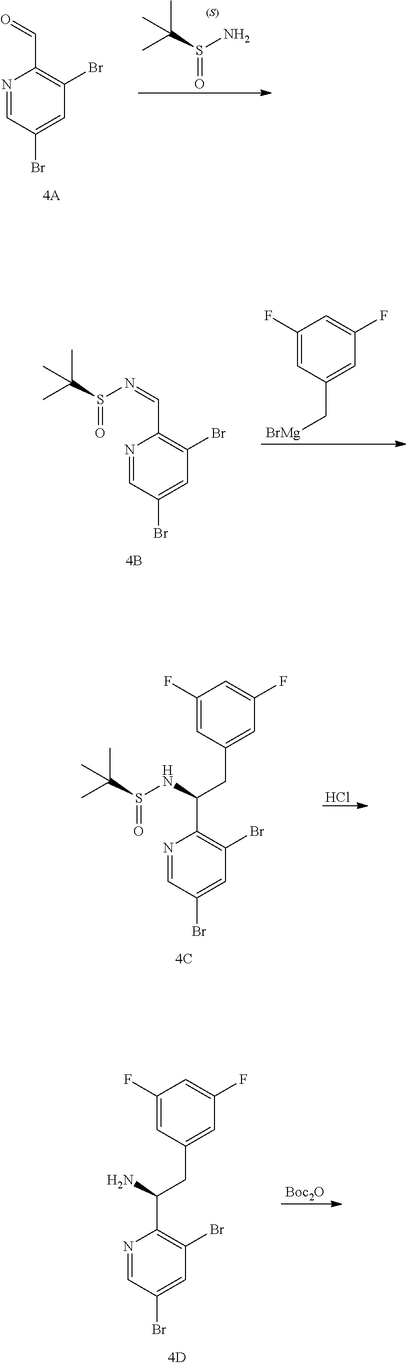

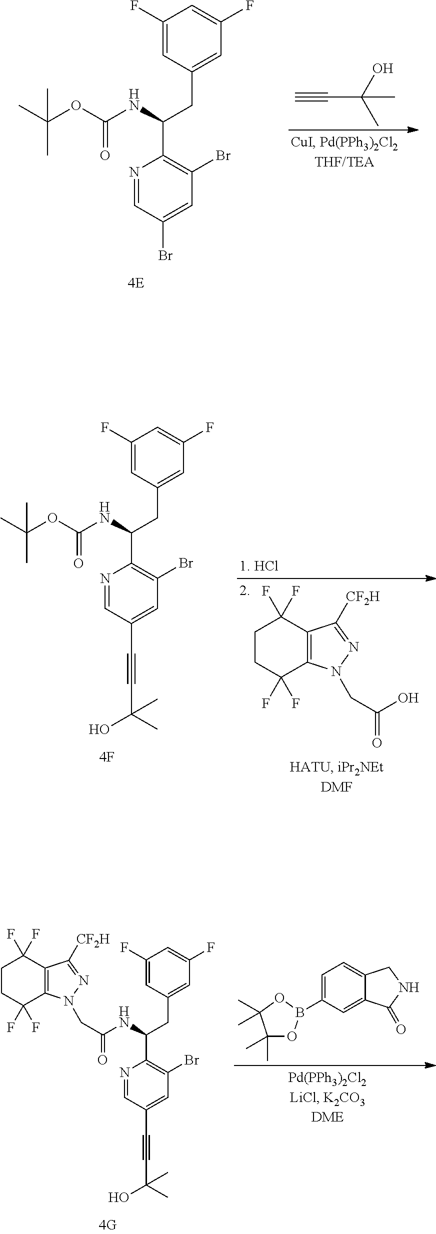

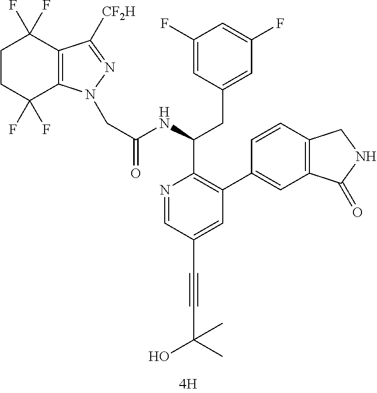 [SMILES: CC(C)(O)C#Cc1cnc([C@H](Cc2cc(F)cc(F)c2)NC(=O)Cn2nc(C(F)F)c3c2C(F)(F)CCC3(F)F)c(-c2ccc3c(c2)C(=O)NC3)c1]

### Synthesis of (S)—N-((3,5-dibromopyridin-2-yl)methylene)-2-methylpropane-2-sulfinamide (4B)

To 3,5-dibromopicolinaldehyde (1.9 g, 7.17 mmol) in DCM (30 mL) was added (S)-2-methylpropane-2-sulfinamide (870 mg, 7.17 mmol) and CuSO₄ (2.29 g, 14.3 mmol). The reaction mixture was stirred for 15 h. Solids were filtered over celite. The solvents were removed in vacuo and the residue purified by column chromatography on silica to provide 2.6 g of the title compound. MS (m/z) 368.9 [M+H]⁺.

### Synthesis of (S)—N—((S)-1-(3,5-dibromopyridin-2-yl)-2-(3,5-difluorophenyl)ethyl)-2-methylpropane-2-sulfinamide (4C)

(S)—N-((3,5-dibromopyridin-2-yl)methylene)-2-methylpropane-2-sulfinamide (2.6 g, 7.1 mmol) was dissolved in THF (24 mL) and cooled to −78° C. (3,5-difluorobenzyl)magnesium bromide (34 mL, 0.25 M in Et₂O) was added dropwise. The reaction was stirred at −78° C. for 3 hr then let warm to 0° C. and quenched. The reaction was partitioned between EtOAc and aq. NH₄Cl. The organics were separated, dried, and removed in vacuo. The residue purified by column chromatography on silica to provide the title compound. MS (m/z) 496.6 [M+H]⁺.

### Synthesis of (S)-1-(3,5-dibromopyridin-2-yl)-2-(3,5-difluorophenyl)ethanamine (4D)

To (S)—N—((S)-1-(3,5-dibromopyridin-2-yl)-2-(3,5-difluorophenyl)ethyl)-2-methylpropane-2-sulfinamide (650 mg) dissolved in DCM (3 mL) was added 4N HCl in dioxanes (4 mL). The reaction was stirred for 2 hr at ambient temperature. Solvents were removed in vacuo and the crude desired product was used without further purification. MS (m/z) 393.0 [M+H]⁺.

### Synthesis of (S)-tert-butyl 1-(3,5-dibromopyridin-2-yl)-2-(3,5-difluorophenyl)ethylcarbamate (4E)

(S)-1-(3,5-Dibromopyridin-2-yl)-2-(3,5-difluorophenyl)ethanamine (780 mg, 1.84 mmol) was combined with di-tert-butyl dicarbonate (400 mg, 1.84 mmol) and TEA (515 μL, 3.7 mmol) in DCM (9 mL). The reaction was stirred for 2 hr at ambient temperature. The reaction was partitioned between EtOAc and H₂O. The organics were separated, dried, and removed in vacuo. The residue purified by column chromatography on silica to provide the title compound. MS (m/z) 492.9 [M+H]⁺.

### Synthesis of (S)-tert-butyl 1-(3-bromo-5-(3-hydroxy-3-methylbut-1-ynyl)pyridin-2-yl)-2-(3,5-difluorophenyl)ethylcarbamate (4F)

To (S)-tert-butyl 1-(3,5-dibromopyridin-2-yl)-2-(3,5-difluorophenyl)ethylcarbamate (140 mg, 0.29 mmol) in THF (18 mL) was added 2-methylbut-3-yn-2-ol (42 μL, 0.43 mmol), TEA (0.9 mL), Pd(PPh₃)₂Cl₂ (30 mg) and CuI (16 mg). The reaction was stirred for 2 hr at ambient temperature and then partitioned between EtOAc and H₂O. The organics were separated, dried, and removed in vacuo. The residue purified by column chromatography on silica to provide the title compound as a mixture with 4E which was used in the next step. MS (m/z) 496.7 [M+H]⁺.

### Synthesis of (S)—N-(1-(3-bromo-5-(3-hydroxy-3-methylbut-1-ynyl)pyridin-2-yl)-2-(3,5-difluorophenyl)ethyl)-2-(3-(difluoromethyl)-4,4,7,7-tetrafluoro-4,5,6,7-tetrahydro-1H-indazol-1-yl)acetamide (4G)

A mixture of (S)-tert-butyl 1-(3-bromo-5-(3-hydroxy-3-methylbut-1-ynyl)pyridin-2-yl)-2-(3,5-difluorophenyl)ethylcarbamate and (S)-tert-butyl 1-(3,5-dibromopyridin-2-yl)-2-(3,5-difluorophenyl)ethylcarbamate (105 mg) obtained from the previous step was dissolved in DCM (3 mL) and treated with 4N HCl in dioxanes (4 mL). The reaction was stirred for 2 hr then solvents removed in vacuo. The residue purified by column chromatography on silica to provide 18 mg of (S)-4-(6-(1-amino-2-(3,5-difluorophenyl)ethyl)-5-bromopyridin-3-yl)-2-methylbut-3-yn-2-ol (MS (m/z) 395.0 [M+H]⁺). To (S)-4-(6-(1-amino-2-(3,5-difluorophenyl)ethyl)-5-bromopyridin-3-yl)-2-methylbut-3-yn-2-ol (18 mg, 0.046 mmol) in DMF (1 mL) was added 2-(3-(difluoromethyl)-4,4,7,7-tetrafluoro-4,5,6,7-tetrahydro-1H-indazol-1-yl)acetic acid (15 mg, 0.05 mmol), iPr₂NEt (17 μL, 0.1 mmol) and HATU (26 mg, 0.07 mmol). The reaction was stirred 30 min and then partitioned between EtOAc and H₂O. The organics were separated, dried, and removed in vacuo. The crude product was used directly in the next reaction. MS (m/z) 679.2[M+H]⁺.

### Synthesis of (S)-2-(3-(difluoromethyl)-4,4,7,7-tetrafluoro-4,5,6,7-tetrahydro-1H-indazol-1-yl)-N-(2-(3,5-difluorophenyl)-1-(5-(3-hydroxy-3-methylbut-1-ynyl)-3-(3-oxoisoindolin-5-yl)pyridin-2-yl)ethyl)acetamide (4H)

To (S)—N-(1-(3-bromo-5-(3-hydroxy-3-methylbut-1-ynyl)pyridin-2-yl)-2-(3,5-difluorophenyl)ethyl)-2-(3-(difluoromethyl)-4,4,7,7-tetrafluoro-4,5,6,7-tetrahydro-1H-indazol-1-yl)acetamide (16 mg, 0.02 mmol) in DME (0.7 mL) was added 6-(4,4,5,5-tetramethyl-1,3,2-dioxaborolan-2-yl)isoindolin-1-one (7 mg, 0.03 mmol), Pd(PPh₃)₂Cl₂ (2 mg), LiCl (1 mg), and aq 2M K₂CO₃ (30 μL). The reaction was heated in a microwave reactor to 150° C. for 20 min. The reaction was purified by RP HPLC to provide the desired product. ¹H NMR (400 MHz, Methanol-d₄) δ 8.69 (d, 1H), 7.62-7.49 (m, 2H), 7.43 (s, 1H), 7.28 (s, 1H), 6.98-6.58 (m, 4H), 6.26 (d, 2H), 5.34 (d, 2H), 5.18 (s, 1H), 5.05 (s, 2H), 4.48 (s, 2H), 3.02 (t, J=7.5 Hz, 3H), 2.49 (s, 7H), 1.56 (s, 5H). MS (m/z) 732.1 [M+H]⁺.

### Example 5

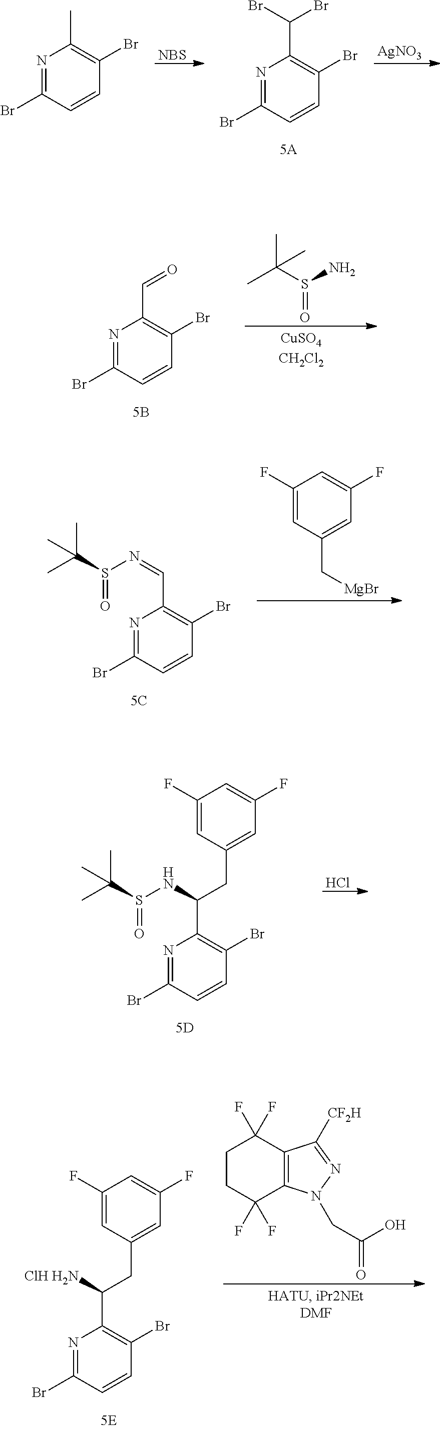

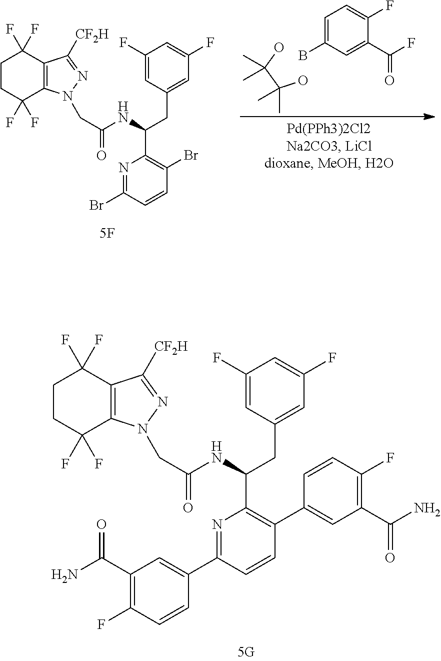

### Synthesis of 3,6-dibromo-2-(dibromomethyl)pyridine (5A)

To a solution of 3,6-dibromo-2-methylpyridine (5.2 g, 21 mmol) in CCl₄ (50 mL) was added N-bromosuccinimide (7.57 g, 42 mmol) and 2,2′-azobis(2-methylpropionitrile) (0.70 g, 4.3 mmol). The mixture was heated at 80° C. overnight and cooled to room temperature. The solid was removed by filtration and the filtrate was concentrated under reduced pressure. The product (5A) was obtained after flash chromatography eluding with 0-10 percent EtOAc in hexane (7.36 g). MS (m/z): 409.66 [M+H]⁺

### Synthesis of 3,6-dibromopicolinaldehyde (5B)

A solution of silver nitrate (7.6 g, 45 mmol) in water (24 mL) was added dropwise to a solution of 5A (7.36 g, 18 mmol) in refluxing EtOH (90 mL). The mixture was stirred at 80° C. for 5 hours. After the mixture was cooled to room temperature, it was diluted with water (100 mL), extracted with EtOAc (3 times), dried over Na₂SO₄, filtered and concentrated under reduced pressure. The crude product (5B, 4.6 G) was directly used for next step. MS (m/z): 265.96. [M+H]⁺

### Synthesis of (S,Z)—N-((3,6-dibromopyridin-2-yl)methylene)-2-methylpropane-2-sulfinamide (5C)

The title compound (5C) was prepared according to the method presented for the synthesis of compound 4B of Example 4 utilizing 5B. MS (m/z) 368.86 [M+H]⁺

### Synthesis of (S)—N—((S)-1-(3,6-dibromopyridin-2-yl)-2-(3,5-difluorophenyl)ethyl)-2-methylpropane-2-sulfinamide (SD)

The title compound (5D) was prepared according to the method presented for the synthesis of compound 4C of Example 4 utilizing 5C. MS (m/z) 496.99 [M+H]⁺

### Synthesis of (S)-1-(3,6-dibromopyridin-2-yl)-2-(3,5-difluorophenyl)ethanamine hydrochloride (5E)

The title compound (5E) was prepared according to the method presented for the synthesis of compound 4D of Example 4 utilizing 5D. MS (m/z) 393.29 [M+H]⁺

### Synthesis of (S)—N-(1-(3,6-dibromopyridin-2-yl)-2-(3,5-difluorophenyl)ethyl)-2-(3-(difluoromethyl)-4,4,7,7-tetrafluoro-4,5,6,7-tetrahydro-1H-indazol-1-yl)acetamide (5F)

The title compound (5F) was prepared according to the method presented for the synthesis of compound 10A of Example 10 utilizing 5E. MS (m/z) 676.96 [M+H]⁺.

### Synthesis of (S)-5,5′-(6-(1-(2-(3-(difluoromethyl)-4,4,7,7-tetrafluoro-4,5,6,7-tetrahydro-1H-indazol-1-yl)acetamido)-2-(3,5-difluorophenyl)ethyl)pyridine-2,5-diyl)bis(2-fluorobenzamide) (5G)

In a microwave tube was charged with 5F (100 mg, 0.15 mmol), (3-carbamoyl-4-fluorophenyl)boronic acid (81 mg, 0.45 mmol), LiCl (19 mg, 0.45 mmol), Na₂CO₃ (50 mg, 0.6 mmol) and 5 mg of Pd(PPh₃)₂Cl₂. To the mixture was added 1.4 mL of 1,4-dioxane/methanol/H₂O (5/1/1). The mixture was heated up to 170° C. for 15 min in a Microwave Synthesizer. After cooled down and filtered through a syringe filter, purified on reverse phase HPLC eluting with acetonitrile and water (with 0.1% TFA) to afford the title compound. ¹H NMR (400 MHz, CD₃OD) δ 8.90 (d, J=8.6 Hz, 1H), 8.74 (dd, J=7.2, 2.4 Hz, 1H), 8.51-8.30 (m, 1H), 7.91 (d, J=8.1 Hz, 1H), 7.64 (d, J=8.1 Hz, 1H), 7.41 (m, 2H), 7.23 (dd, J=10.7, 8.5 Hz, 1H), 7.02-6.49 (m, 2H), 6.35 (d, J=6.2 Hz, 2H), 5.45 (m, 1H), 5.16-5.02 (m, 2H), 3.23-2.97 (m, 2H), 2.49 (m, 4H). MS (m/z) 793.19 [M+H]⁺.

### Example 6

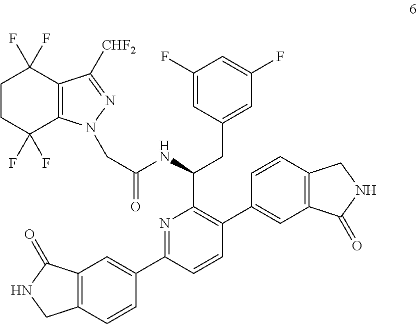 [SMILES: O=C(Cn1nc(C(F)F)c2c1C(F)(F)CCC2(F)F)N[C@@H](Cc1cc(F)cc(F)c1)c1nc(-c2ccc3c(c2)C(=O)NC3)ccc1-c1ccc2c(c1)C(=O)NC2]

### Synthesis of (S)—N-(1-(3,6-bis(3-oxoisoindolin-5-yl)pyridin-2-yl)-2-(3,5-difluorophenyl)ethyl)-2-(3-(difluoromethyl)-4,4,7,7-tetrafluoro-4,5,6,7-tetrahydro-1H-indazol-1-yl)acetamide (6)

The title compound (6) was prepared according to the method presented for the synthesis of compound 5G of Example 5 utilizing 6-(4,4,5,5-tetramethyl-1,3,2-dioxaborolan-2-yl)isoindolin-1-one and 5F. ¹H NMR (400 MHz, CD₃OD) δ 8.84 (d, J=8.1 Hz, 1H), 8.72 (s, 1H), 8.49 (d, J=7.9 Hz, 1H), 7.98 (d, J=8.1 Hz, 2H), 7.72 (dd, J=23.8, 8.0 Hz, 2H), 7.60 (d, J=7.9 Hz, 1H), 7.49 (d, J=8.0 Hz, 1H), 7.39 (s, 1H), 6.97-6.57 (m, 2H), 6.33 (m, 2H), 5.49 (m, 2H), 5.10 (s, 2H), 4.57 (s, 2H), 4.49 (s, 2H), 3.24-2.95 (m, 2H), 2.47 (m, 4H). MS (m/z) 781.02[M+H]⁺.

### Example 7

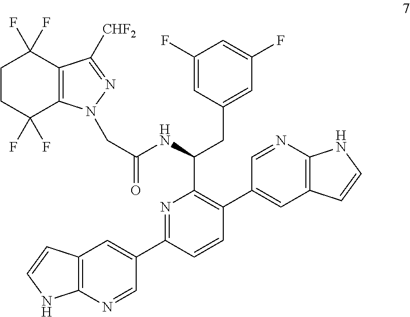 [SMILES: O=C(Cn1nc(C(F)F)c2c1C(F)(F)CCC2(F)F)N[C@@H](Cc1cc(F)cc(F)c1)c1nc(-c2cnc3[nH]ccc3c2)ccc1-c1cnc2[nH]ccc2c1]

### Synthesis of (S)—N-(1-(3,6-bis(1H-pyrrolo[2,3-b]pyridin-5-yl)pyridin-2-yl)-2-(3,5-difluorophenyl)ethyl)-2-(3-(difluoromethyl)-4,4,7,7-tetrafluoro-4,5,6,7-tetrahydro-1H-indazol-1-yl)acetamide (7)

The title compound (7) was prepared according to the method presented for the synthesis of compound 5G of Example 5 utilizing 6 5-(4,4,5,5-tetramethyl-1,3,2-dioxaborolan-2-yl)-1H-pyrrolo[2,3-b]pyridine and 5F. ¹H NMR (400 MHz, CD₃OD) δ 9.23-9.17 (m, 2H), 9.04 (d, J=8.1 Hz, 1H), 8.03 (m, 3H), 7.75 (d, J=8.1 Hz, 1H), 7.61 (dd, J=7.3, 3.5 Hz, 2H), 6.93-6.52 (m, 4H), 6.34 (d, J=6.2 Hz, 2H), 5.45 (m, 1H), 5.10 (m, 2H), 3.27-3.06 (m, 2H), 2.48 (m, 4H). MS (m/z) 751.22 [M+H]⁺.

### Example 8

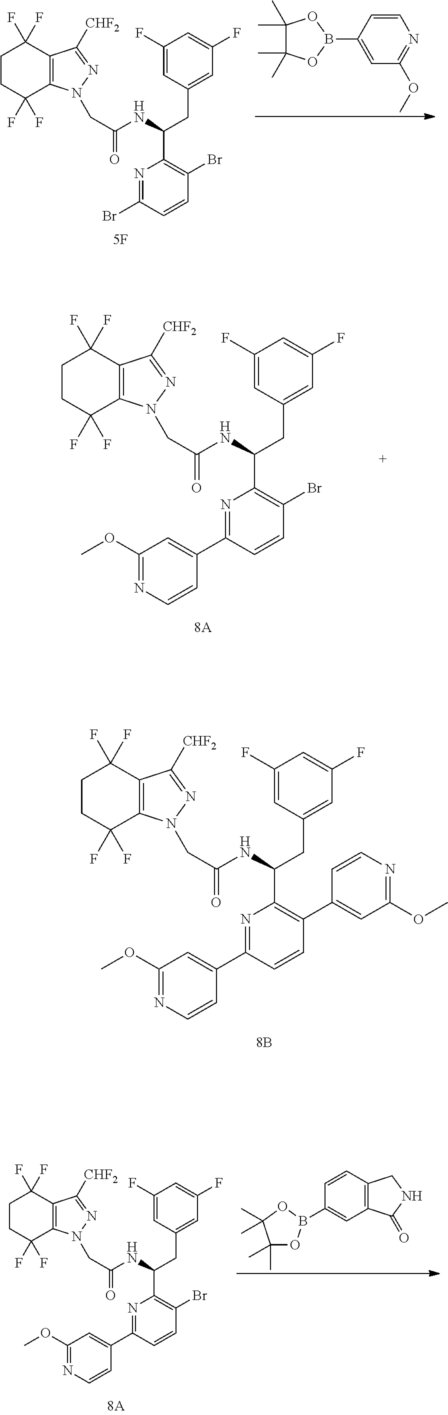

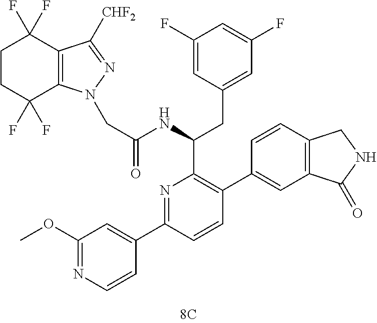 [SMILES: COc1cc(-c2ccc(-c3ccc4c(c3)C(=O)NC4)c([C@H](Cc3cc(F)cc(F)c3)NC(=O)Cn3nc(C(F)F)c4c3C(F)(F)CCC4(F)F)n2)ccn1]

### Synthesis of (S)—N-(1-(5-bromo-2′-methoxy-[2,4′-bipyridin]-6-yl)-2-(3,5-difluorophenyl)ethyl)-2-(3-(difluoromethyl)-4,4,7,7-tetrafluoro-4,5,6,7-tetrahydro-1H-indazol-1-yl)acetamide (8A) and (S)—N-(1-(2′,5′-di(methoxy-[2,4′-bipyridin]-6-yl))-2-(3,5-difluorophenyl)ethyl)-2-(3-(difluoromethyl)-4,4,7,7-tetrafluoro-4,5,6,7-tetrahydro-1H-indazol-1-yl)acetamide (8B)

The title compounds (8A and 8B) were prepared according to the method presented for the synthesis of compound 5G of Example 5 utilizing 2-methoxy-4-(4,4,5,5-tetramethyl-1,3,2-dioxaborolan-2-yl)pyridine (2 equiv.) and 5F.

### Synthesis of (S)-2-(3-(difluoromethyl)-4,4,7,7-tetrafluoro-4,5,6,7-tetrahydro-1H-indazol-1-yl)-N-(2-(3,5-difluorophenyl)-1-(2′-methoxy-5-(3-oxoisoindolin-5-yl)-[2,4′-bipyridin]-6-yl)ethyl)acetamide (8C)

The title compound (8C) was prepared according to the method presented for the synthesis of compound 4H of Example 4 utilizing 8A. ¹H NMR (400 MHz, CD₃OD) δ 8.85 (d, J=8.1 Hz, 1H), 8.33 (d, J=5.7 Hz, 1H), 8.06 (d, J=8.0 Hz, 1H), 7.91 (d, J=5.7 Hz, 1H), 7.82 (s, 1H), 7.74 (d, J=8.0 Hz, 1H), 7.61 (d, J=6.3 Hz, 1H), 7.53 (d, J=7.8 Hz, 1H), 7.38 (s, 1H), 6.91-6.44 (m, 2H), 6.29 (d, J=6.3 Hz, 2H), 5.51 (dd, J=14.8, 8.2 Hz, 1H), 5.18-4.98 (m, 2H), 4.50 (s, 2H), 4.09 (s, 3H), 3.12 (m, 2H), 2.49 (m, 4H). MS (m/z) 757.25 [M+H]⁺.

### Example 9

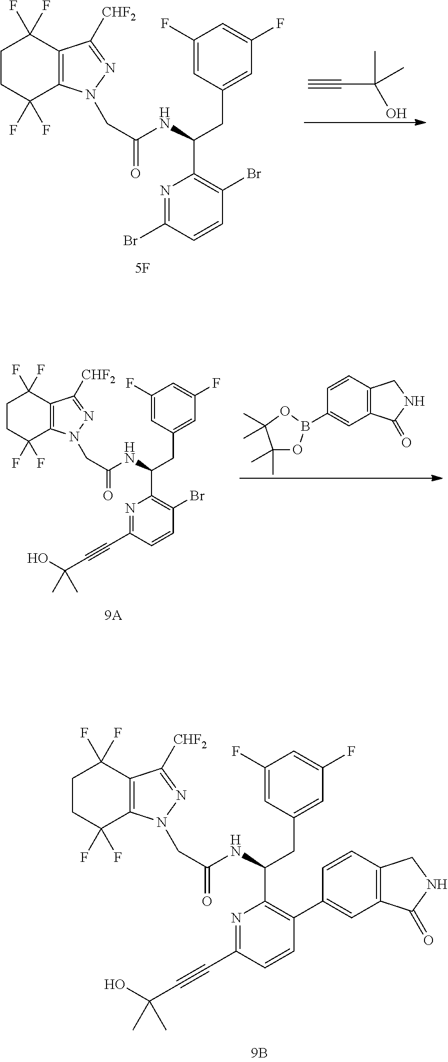

### Synthesis of (S)—N-(1-(3-bromo-6-(3-hydroxy-3-methylbut-1-yn-1-yl)pyridin-2-yl)-2-(3,5-difluorophenyl)ethyl)-2-(3-(difluoromethyl)-4,4,7,7-tetrafluoro-4,5,6,7-tetrahydro-1H-indazol-1-yl)acetamide (9A)

The title compound (9A) was prepared according to the method presented for the synthesis of compound 4F of Example 4 utilizing 2-methylbut-3-yn-2-ol and 5F. MS (m/z) 681.17 [M+H]⁺.

### Synthesis of (S)-2-(3-(difluoromethyl)-4,4,7,7-tetrafluoro-4,5,6,7-tetrahydro-1H-indazol-1-yl)-N-(2-(3,5-difluorophenyl)-1-(6-(3-hydroxy-3-methylbut-1-yn-1-yl)-3-(3-oxoisoindolin-5-yl)pyridin-2-yl)ethyl)acetamide (9B)

The title compound (9B) was prepared according to the method presented for the synthesis of compound 4H of Example 4 utilizing 9A. ¹H NMR (400 MHz, CD₃OD) δ 7.63-7.53 (m, 2H), 7.50-7.40 (m, 2H), 7.30 (s, 1H), 6.95-6.56 (m, 2H), 6.28 (d, J=6.3 Hz, 2H), 5.39 (t, J=7.4 Hz, 1H), 5.05 (s, 2H), 4.48 (s, 2H), 3.13-2.91 (m, 2H), 2.66-2.35 (m, 4H), 1.61 (s, 6H). MS (m/z) 732.23 [M+H]⁺.

### Example 10

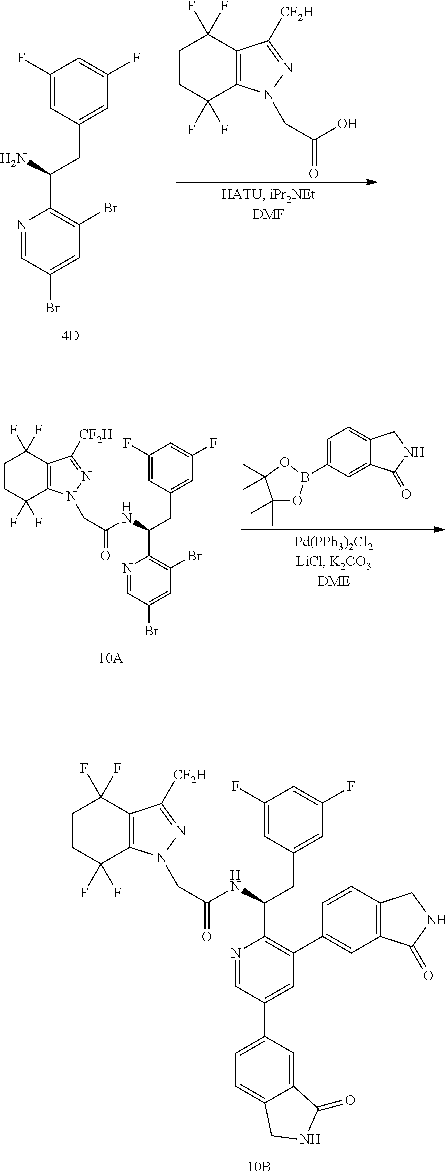

### Synthesis of (S)—N-(1-(3,5-dibromopyridin-2-yl)-2-(3,5-difluorophenyl)ethyl)-2-(3-(difluoromethyl)-4,4,7,7-tetrafluoro-4,5,6,7-tetrahydro-1H-indazol-1-yl)acetamide (100A)

To (S)-1-(3,5-dibromopyridin-2-yl)-2-(3,5-difluorophenyl)ethanamine (380 mg, 0.97 mmol) dissolved in DMF (10 mL) was added iPr₂Net (350 μL, 2 mmol) and 2-(3-(difluoromethyl)-4,4,7,7-tetrafluoro-4,5,6,7-tetrahydro-1H-indazol-1-yl)acetic acid (293 mg, 0.97 mmol). HATU (442 mg, 1.16 mmol) was added and the reaction stirred for 30 min. The reaction was partitioned between EtOAc and H₂O. The organics were separated, dried, and removed in vacuo. The residue purified by column chromatography on silica to provide the title compound. MS (m/z) 677.1 [M+H]⁺.

### Synthesis of (S)—N-(1-(3,5-bis(3-oxoisoindolin-5-yl)pyridin-2-yl)-2-(3,5-difluorophenyl)ethyl)-2-(3-(difluoromethyl)-4,4,7,7-tetrafluoro-4,5,6,7-tetrahydro-1H-indazol-1-yl)acetamide (10B)

To (S)—N-(1-(3,5-dibromopyridin-2-yl)-2-(3,5-difluorophenyl)ethyl)-2-(3-(difluoromethyl)-4,4,7,7-tetrafluoro-4,5,6,7-tetrahydro-1H-indazol-1-yl)acetamide (50 mg, 0.074 mmol) in DME (0.8 mL) and DMF (0.2 mL) was added 6-(4,4,5,5-tetramethyl-1,3,2-dioxaborolan-2-yl)isoindolin-1-one (48 mg, 0.19 mmol), Pd(PPh₃)₂Cl₂ (5 mg), LiCl (2 mg), and aq 2M K₂CO₃ (110 μL). The reaction was heated in a microwave reactor to 150° C. for 20 min. The reaction was purified by RP HPLC to provide the desired product. ¹H NMR (400 MHz, Methanol-d₄) δ 9.02 (d, 1H), 8.11 (d, 1H), 7.97 (dd, 1.7 Hz, 1H), 7.89 (d, 1H), 7.72 (d, 1H), 7.67-7.48 (m, 3H), 7.42 (d, 1H), 6.70-6.61 (m, 2H), 6.37-6.30 (m, 2H), 5.44 (t, 1H), 5.07 (s, 2H), 4.51 (d, 4H), 3.18-3.01 (m, 3H), 2.50 (dd, 4H). MS (m/z) 798.1[M+H]⁺.

### Example 11

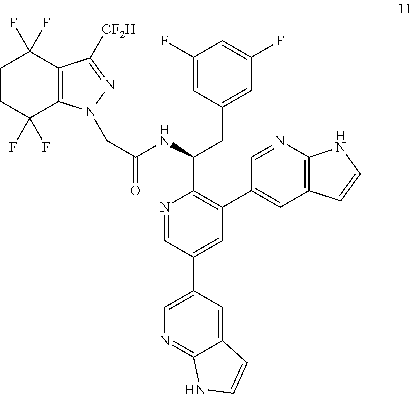 [SMILES: O=C(Cn1nc(C(F)F)c2c1C(F)(F)CCC2(F)F)N[C@@H](Cc1cc(F)cc(F)c1)c1ncc(-c2cnc3[nH]ccc3c2)cc1-c1cnc2[nH]ccc2c1]

### Synthesis of (S)—N-(1-(3,5-di(1H-pyrrolo[2,3-b]pyridin-5-yl)pyridin-2-yl)-2-(3,5-difluorophenyl)ethyl)-2-(3-(difluoromethyl)-4,4,7,7-tetrafluoro-4,5,6,7-tetrahydro-1H-indazol-1-yl)acetamide (11)

The title compound was prepared according to the method presented for the synthesis of 10B of Example 10 utilizing 100A and 5-(4,4,5,5-tetramethyl-1,3,2-dioxaborolan-2-yl)-1H-pyrrolo[2,3-b]pyridine. ¹H NMR (400 MHz, Methanol-d₄) δ 9.10 (s, 1H), 8.62 (s, 1H), 8.56 (s, 1H), 8.14 (s, 1H), 8.05-7.94 (m, 2H), 7.58 (dd, 2H), 6.99-6.61 (m, 4H), 6.36 (d, 2H), 5.47-5.27 (m, 2H), 5.15-5.00 (m, 2H), 3.24-3.01 (m, 3H), 2.66-2.32 (m, 5H). MS (m/z) 751.1[M+H]⁺.

### Example 12

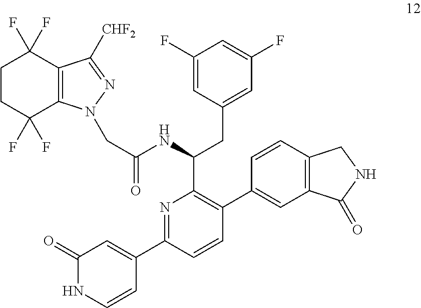 [SMILES: O=C(Cn1nc(C(F)F)c2c1C(F)(F)CCC2(F)F)N[C@@H](Cc1cc(F)cc(F)c1)c1nc(-c2cc[nH]c(=O)c2)ccc1-c1ccc2c(c1)C(=O)NC2]

### Synthesis of (S)-2-(3-(difluoromethyl)-4,4,7,7-tetrafluoro-4,5,6,7-tetrahydro-1H-indazol-1-yl)-N-(2-(3,5-difluorophenyl)-1-(2′-oxo-5-(3-oxoisoindolin-5-yl)-1′,2′-dihydro-[2,4′-bipyridin]-6-yl)ethyl)acetamide (12)

In a microwave tube were charged with (S)-2-(3-(difluoromethyl)-4,4,7,7-tetrafluoro-4,5,6,7-tetrahydro-1H-indazol-1-yl)-N-(2-(3,5-difluorophenyl)-1-(2′-methoxy-5-(3-oxoisoindolin-5-yl)-[2,4′-bipyridin]-6-yl)ethyl)acetamide (8C, 5 mg), HCl in 1,4-dioxane (4N, 0.3 mL) and ethanol (0.3 mL). The mixture was heated up to 100° C. for 20 min in a Microwave Synthesizer. After cooled down, After cooled down, the solvent was removed and the residue was purified on reverse phase HPLC eluting with acetonitrile and water (with 0.1% TFA) to afford the title product. ¹H NMR (400 MHz, CD₃OD) δ 8.88 (m, 1H), 7.96 (d, J=8.0 Hz, 1H), 7.73 (d, J=8.0 Hz, 1H), 7.60 (m, 2H), 7.53 (m, 1H), 7.43 (s, 1H), 7.37 (s, 1H), 7.27 (d, J=6.8 Hz, 1H), 6.96-6.53 (m, 2H), 6.30 (d, J=6.2 Hz, 2H), 5.49 (m, 1H), 5.09 (s, 2H), 4.49 (s, 2H), 3.12 (m, 2H), 2.48 (m, 4H). MS (m/z) 742.99 [M+H]⁺.

### Example 13

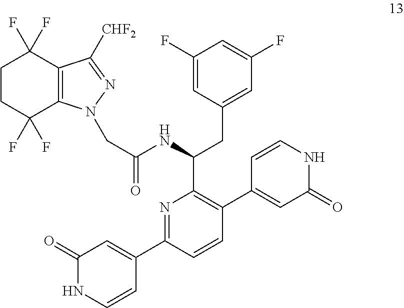 [SMILES: O=C(Cn1nc(C(F)F)c2c1C(F)(F)CCC2(F)F)N[C@@H](Cc1cc(F)cc(F)c1)c1nc(-c2cc[nH]c(=O)c2)ccc1-c1cc[nH]c(=O)c1]

### Synthesis of (S)-2-(3-(difluoromethyl)-4,4,7,7-tetrafluoro-4,5,6,7-tetrahydro-1H-indazol-1-yl)-N-(2-(3,5-difluorophenyl)-1-(2,2″-dioxo-1,1″,2,2″-tetrahydro-[4,2′:5′,4″-terpyridin]-6′-yl)ethyl)acetamide (13)

The title compound (13) was prepared according to the method presented for the synthesis of compound 12 of Example 12 utilizing 8B. ¹H NMR (400 MHz, CD₃OD) δ 8.95 (d, J=7.8 Hz, 1H), 7.95 (d, J=8.1 Hz, 1H), 7.71 (d, J=8.1 Hz, 1H), 7.60 (d, J=6.8 Hz, 1H), 7.43 (d, J=7.2 Hz, 1H), 7.25 (d, J=6.9 Hz, 1H), 6.98-6.61 (m, 2H), 6.45 (d, J=6.3 Hz, 2H), 6.33 (d, J=6.6 Hz, 1H), 6.19 (s, 1H), 5.51 (m, 1H), 5.08 (m, 2H), 3.15 (m, 2H), 2.49 (m, 4H). MS (m/z) 705.00 [M+H]⁺.

### Example 14

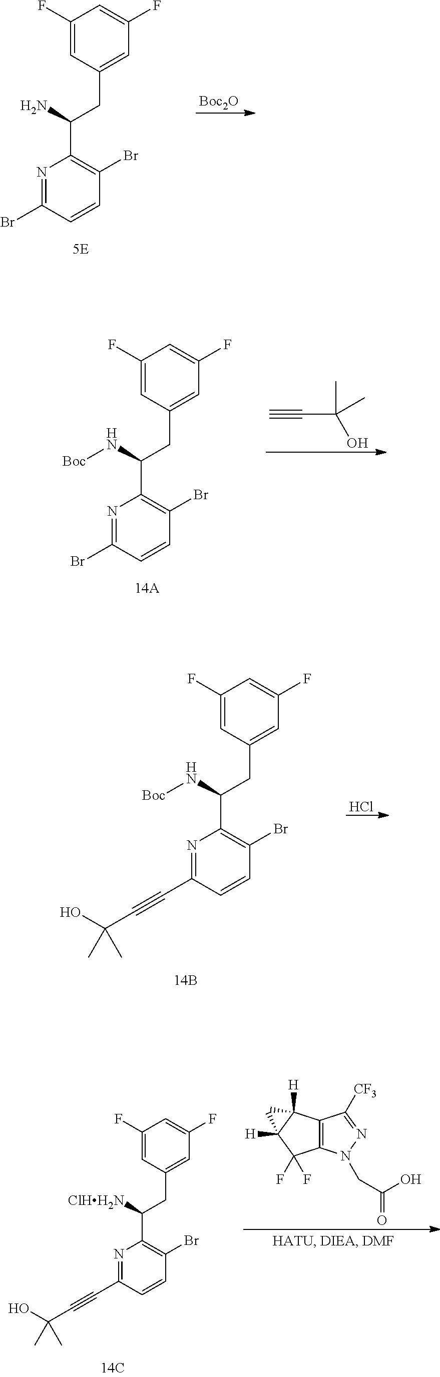

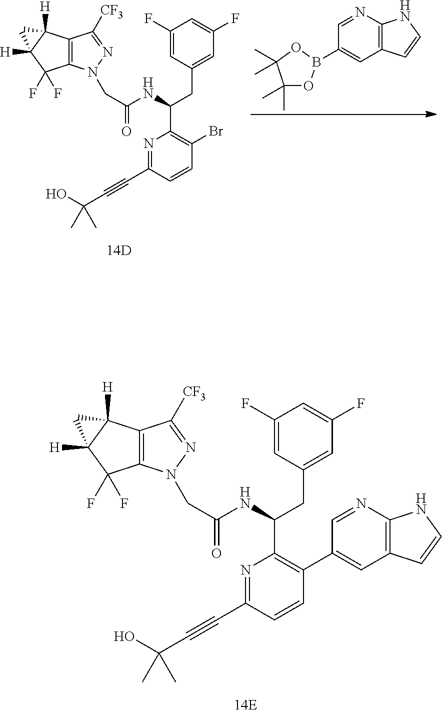 [SMILES: CC(C)(O)C#Cc1ccc(Br)c([C@H](Cc2cc(F)cc(F)c2)NC(=O)Cn2nc(C(F)(F)F)c3c2C(F)(F)[C@@H]2C[C@H]32)n1 ; CC1(C)OB(c2cnc3[nH]ccc3c2)OC1(C)C ; CC(C)(O)C#Cc1ccc(-c2cnc3[nH]ccc3c2)c([C@H](Cc2cc(F)cc(F)c2)NC(=O)Cn2nc(C(F)(F)F)c3c2C(F)(F)[C@@H]2C[C@H]32)n1]

### Synthesis of (S)-tert-butyl 1-(3,6-dibromopyridin-2-yl)-2-(3,5-difluorophenyl)ethylcarbamate (14A)

The title compound was prepared according to the method presented for the synthesis of compound 4E of Example 4 utilizing 5E.

### Synthesis of (S)-tert-butyl (1-(3-bromo-6-(3-hydroxy-3-methylbut-1-yn-1-yl)pyridin-2-yl)-2-(3,5-difluorophenyl)ethyl)carbamate (14B)

The title compound (14B) was prepared according to the method presented for the synthesis of compound 4F of Example 4 utilizing 2-methylbut-3-yn-2-ol and (S)-tert-butyl (1-(3,6-dibromopyridin-2-yl)-2-(3,5-difluorophenyl)ethyl)carbamate. MS (m/z) 496.90 [M+H]⁺.

### Synthesis of (S)-4-(6-(1-amino-2-(3,5-difluorophenyl)ethyl)-5-bromopyridin-2-yl)-2-methylbut-3-yn-2-ol compound with 2-methylbut-3-yn-2-ol (1:1) hydrochloride (14C)

The title compound (14C) was prepared according to the method presented for the synthesis of compound 4G of Example 4 utilizing 14B. MS (m/z) 397.09 [M+H]⁺.

### Synthesis of N—((S)-1-(3-bromo-6-(3-hydroxy-3-methylbut-1-yn-1-yl)pyridin-2-yl)-2-(3,5-difluorophenyl)ethyl)-2-((3b-5,4-aR)-5,5-difluoro-3-(trifluoromethyl)-3b,4,4a,5-tetrahydro-1H-cyclopropa[3,4]cyclopenta[1,2-c]pyrazol-1-yl)acetamide (14D)

The title compound (14D) was prepared according to the method presented for the synthesis of compound 4G of Example 4 utilizing 2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetic acid and 14C. MS (m/z) 659.23 [M+H]⁺.

### Synthesis of 2-((3bS,4aR)-5,5-difluoro-3-(trifluoromethyl)-3b,4,4a,5-tetrahydro-1H-cyclopropa[3,4]cyclopenta[1,2-c]pyrazol-1-yl)-N—((S)-2-(3,5-difluorophenyl)-1-(6-(3-hydroxy-3-methylbut-1-yn-1-yl)-3-(1-oxoisoindolin-4-yl)pyridin-2-yl)ethyl)acetamide (14E)

The title compound (14E) was prepared according to the method presented for the synthesis of compound 4H of Example 4 utilizing 5-(4,4,5,5-tetramethyl-1,3,2-dioxaborolan-2-yl)-1H-pyrrolo[2,3-b]pyridine and 14D. ¹H NMR (400 MHz, CD₃OD) δ 8.92 (d, J=8.7 Hz, 1H), 8.00 (s, 2H), 7.85 (s, 1H), 7.59 (m, 2H), 7.48 (d, J=7.9 Hz, 1H), 6.77-6.56 (m, 2H), 6.28 (d, J=6.3 Hz, 2H), 5.33 (m, 1H), 4.87 (s, 2H), 3.17-2.99 (m, 4H), 2.48 (m, 4H), 1.6 (s, 6H), 1.40 (m, 1H), 1.10 (m, 1H). MS (m/z) 697.28 [M+H]⁺.

### Example 15

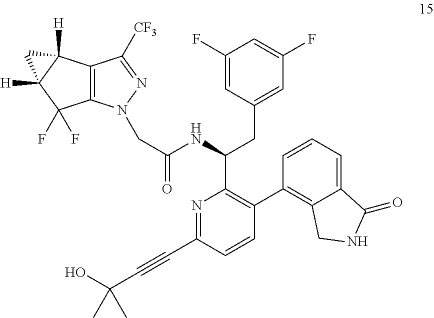 [SMILES: CC(C)(O)C#Cc1ccc(-c2cccc3c2CNC3=O)c([C@H](Cc2cc(F)cc(F)c2)NC(=O)Cn2nc(C(F)(F)F)c3c2C(F)(F)[C@@H]2C[C@H]32)n1]

### Synthesis of 2-((3bS,4aR)-5,5-difluoro-3-(trifluoromethyl)-3b,4,4a,5-tetrahydro-1H-cyclopropa[3,4]cyclopenta[1,2-c]pyrazol-1-yl)-N—((S)-2-(3,5-difluorophenyl)-1-(6-(3-hydroxy-3-methylbut-1-yn-1-yl)-3-(1-oxoisoindolin-4-yl)pyridin-2-yl)ethyl)acetamide (15)

The title compound (15) was prepared according to the method presented for the synthesis of compound 4H of Example 4 utilizing 14D and 2,3-dihydro-1H-isoindol-1-one-4-boronic acid pinacol ester. ¹H NMR (400 MHz, CD₃OD) δ 7.82 (m, 1H), 7.53 (m, 4H), 6.78 (m, 1H), 6.30 (m, 2H), 5.35 (m, 1H), 4.83 (m, 2H), 4.17 (m, 2H), 3.16-3.04 (m, 1H), 2.98 (m, 1H), 2.48 (m, 2H), 1.53 (s, 6H), 1.43 (m, 1H), 1.08 (m, 1H). MS (m/z) 712.18 [M+H]⁺.

### Example 16

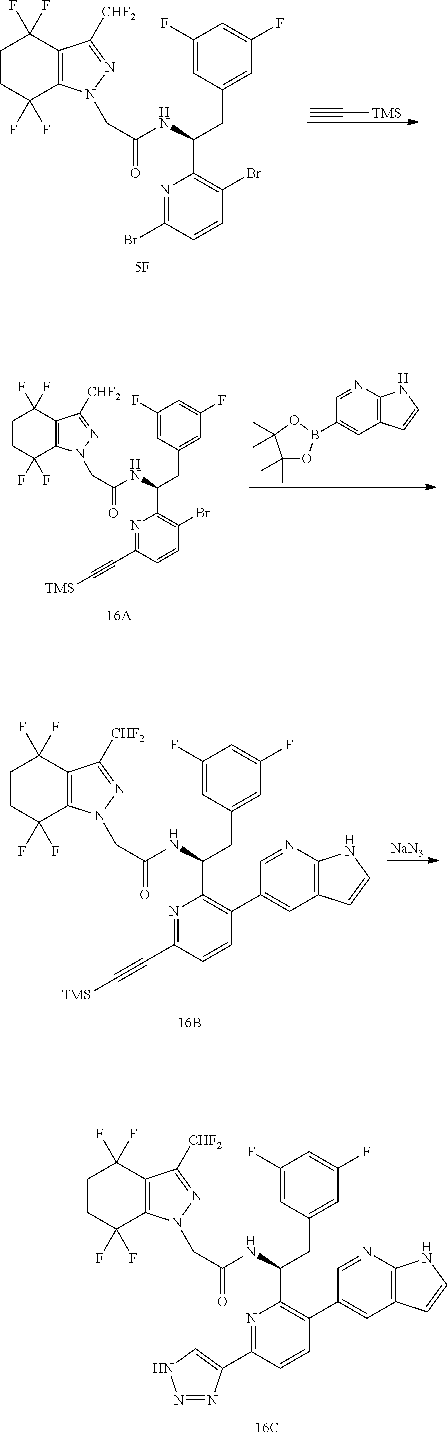

### Synthesis of (S)—N-(1-(3-bromo-6-((trimethylsilyl)ethynyl)pyridin-2-yl)-2-(3,5-difluorophenyl)ethyl)-2-(3-(difluoromethyl)-4,4,7,7-tetrafluoro-4,5,6,7-tetrahydro-1H-indazol-1-yl)acetamide (16A)

The title compound (16A) was prepared according to the method presented for the synthesis of compound 4F of Example 4 utilizing ethynyltrimethylsilane and 5F. MS (m/z) 694.59 [M+H]⁺.

### Synthesis of (S)—N-(1-(3-(1H-pyrrolo[2,3-b]pyridin-5-yl)-6-((trimethylsilyl)ethynyl)pyridin-2-yl)-2-(3,5-difluorophenyl)ethyl)-2-(3-(difluoromethyl)-4,4,7,7-tetrafluoro-4,5,6,7-tetrahydro-1H-indazol-1-yl)acetamide (16B)

The title compound (16B) was prepared according to the method presented for the synthesis of compound 4H of Example 4 utilizing 5-(4,4,5,5-tetramethyl-1,3,2-dioxaborolan-2-yl)-1H-pyrrolo[2,3-b]pyridine and 16A. MS (m/z) 731.22 [M+H]⁺.

### Synthesis of (S)—N-(1-(3-(1H-pyrrolo[2,3-b]pyridin-5-yl)-6-(1H-1,2,3-triazol-4-yl)pyridin-2-yl)-2-(3,5-difluorophenyl)ethyl)-4,4,7,7-tetrafluoro-4,5,6,7-tetrahydro-1H-indazol-1-yl)acetamide (16C)

Compound 16B (75 mg, 0.1 mmol), NaN₃ (13 mg, 0.2 mmol) and NH₄Cl (5 mg, 0.1 mmol) were dissolved in DMF (0.5 mL) and stirred at 100° C. for overnight. The reaction mixture was cooled down to room temperature and diluted with water and extracted with EtOAc. The organic phase was dried (Na₂SO₄), filtered and concentrated. The crude material was purified on reverse phase HPLC eluting with acetonitrile and water (with 0.1% TFA) to afford the title product. ¹H NMR (400 MHz, CD₃OD) δ 9.01 (d, J=7.7 Hz, 1H), 8.54 (s, 1H), 7.99 (m, 3H), 7.73 (d, J=8.0 Hz, 1H), 7.59 (d, J=3.5 Hz, 1H), 6.97-6.55 (m, 3H), 6.31 (d, J=6.3 Hz, 2H), 5.45 (m, 1H), 5.11 (s, 2H), 3.13 (m, 2H), 2.49 (m, 4H). MS (m/z) 702.02 [M+H]⁺.

### Example 17

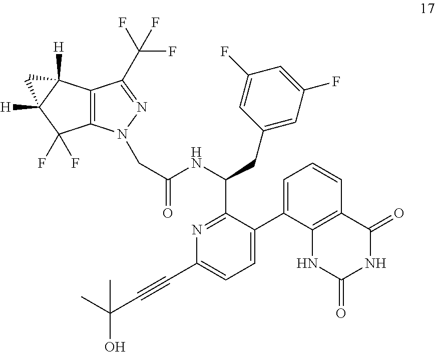 [SMILES: CC(C)(O)C#Cc1ccc(-c2cccc3c(=O)[nH]c(=O)[nH]c23)c([C@H](Cc2cc(F)cc(F)c2)NC(=O)Cn2nc(C(F)(F)F)c3c2C(F)(F)[C@@H]2C[C@H]32)n1]

### Synthesis of 2-((3bS,4aR)-5,5-difluoro-3-(trifluoromethyl)-3b,4,4a,5-tetrahydro-1H-cyclopropa[3,4]cyclopenta[1,2-c]pyrazol-1-yl)-N—((S)-2-(3,5-difluorophenyl)-1-(3-(2,4-dioxo-1,2,3,4-tetrahydroquinazolin-8-yl)-6-(3-hydroxy-3-methylbut-1-ynyl)pyridin-2-yl)ethyl)acetamide (17)

The title compound was prepared according to the method presented for the synthesis of compound 4F of Example 4 utilizing 8-(4,4,5,5-tetramethyl-1,3,2-dioxaborolan-2-yl)quinazoline-2,4(1H,3H)-dione and 14D. ¹H NMR (400 MHz, DMSO) δ 11.36 (d, 1H), 10.12 (d, 1H), 8.87 (m, 1H), 7.98 (d, 1H), 7.75-6.70 (m, 7H), 6.47-6.57 (m, 2H), 4.74-4.50 (m, 2H), 3.01-2.90 (m, 2H), 2.48-2.60 (m, 2H), 1.49 (s, 6H), 1.45-1.24 (m, 1H), 0.96 (m, 1H). MS (m/z) 741.1 [M+H]⁺.

### Example 18

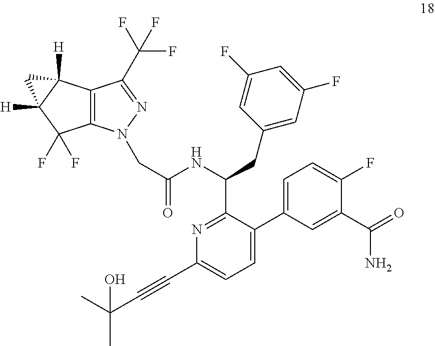 [SMILES: CC(C)(O)C#Cc1ccc(-c2ccc(F)c(C(N)=O)c2)c([C@H](Cc2cc(F)cc(F)c2)NC(=O)Cn2nc(C(F)(F)F)c3c2C(F)(F)[C@@H]2C[C@H]32)n1]

### Synthesis of 5-(2-((S)-1-(2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamido)-2-(3,5-difluorophenyl)ethyl)-6-(3-hydroxy-3-methylbut-1-yn-1-yl)pyridin-3-yl)-2-fluorobenzamide (18)

The title compound was prepared according to the method presented for the synthesis of compound 4F of Example 4 utilizing (3-carbamoyl-4-fluorophenyl)boronic acid and 14D. ¹H NMR (400 MHz, DMSO) δ 9.08 (d, 1H), 7.79-7.14 (m, 8H), 6.92 (m, 1H), 6.62 (d, 2H), 5.12 (m, 1H), 4.77-4.83 (m, 2H), 3.01 (m, 2H), 2.55 (m, 1H), 1.51 (s, 6H), 1.38 (m, 1H), 0.98 (m, 1H). MS (m/z) 718.2 [M+H]⁺.

### Example 19

The following illustrate representative pharmaceutical dosage forms, containing a compound of formula I (‘Compound X’), for therapeutic or prophylactic use in humans.

| (i) Tablet 1 | mg/tablet |
|---|---|
| Compound X = | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |
| (ii) Tablet 2 | mg/tablet |
| Compound X = | 20.0 |
| Microcrystalline cellulose | 410.0 |
| Starch | 50.0 |
| Sodium starch glycolate | 15.0 |
| Magnesium stearate | 5.0 |
| | 500.0 |
| (iii) Capsule | mg/capsule |
| Compound X = | 10.0 |
| Colloidal silicon dioxide | 1.5 |
| Lactose | 465.5 |
| Pregelatinized starch | 120.0 |
| Magnesium stearate | 3.0 |
| | 600.0 |
| (iv) Injection 1 (1 mg/ml) | mg/ml |
| Compound X = (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | |
| (pH adjustment to 7.0-7.5) | q.s. |
| Water for injection | q.s. ad 1 mL |
| (v) Injection 2 (10 mg/ml) | mg/ml |
| Compound X = (free acid form) | 10.0 |
| Monobasic sodium phosphate | 0.3 |
| Dibasic sodium phosphate | 1.1 |
| Polyethylene glycol 400 | 200.0 |
| 1.0N Sodium hydroxide solution | |
| (pH adjustment to 7.0-7.5) | q.s. |
| Water for injection | q.s. ad 1 mL |
| (vi) Aerosol | mg/can |
| Compound X = | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

All references, including publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

The use of the terms “a” and “an” and “the” and similar references in the context of this disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., such as, preferred, preferably) provided herein, is intended merely to further illustrate the content of the disclosure and does not pose a limitation on the scope of the claims. No language in the specification should be construed as indicating any non-claimed element as essential to the practice of the present disclosure.

Alternative embodiments of the claimed disclosure are described herein, including the best mode known to the inventors for practicing the claimed invention. Of these, variations of the disclosed embodiments will become apparent to those of ordinary skill in the art upon reading the foregoing disclosure. The inventors expect skilled artisans to employ such variations as appropriate (e.g., altering or combining features or embodiments), and the inventors intend for the invention to be practiced otherwise than as specifically described herein.

Accordingly, this invention includes all modifications and equivalents of the subject matter recited in the claims appended hereto as permitted by applicable law. Moreover, any combination of the above described elements in all possible variations thereof is encompassed by the invention unless otherwise indicated herein or otherwise clearly contradicted by context.

The use of individual numerical values is stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the disclosed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value or range. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Thus, recitation of ranges of values herein are merely intended to serve as a shorthand method of referring individually to each separate value falling within the range, unless otherwise indicated herein, and each separate value is incorporated into the specification as if it were individually recited herein.

It is to be understood that any ranges, ratios and ranges of ratios that can be formed by, or derived from, any of the data disclosed herein represent further embodiments of the present disclosure and are included as part of the disclosure as though they were explicitly set forth. This includes ranges that can be formed that do or do not include a finite upper and/or lower boundary. Accordingly, a person of ordinary skill in the art most closely related to a particular range, ratio or range of ratios will appreciate that such values are unambiguously derivable from the data presented herein.

1. A compound of formula I: 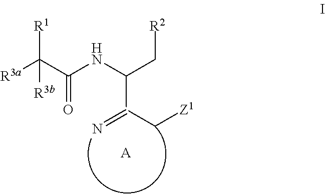
wherein:
A is a 6-membered monocyclic-heteroaryl with one or two nitrogen atoms, wherein the 6-membered monocyclic-heteroaryl is substituted with one Z¹ group at the position shown, one Z² group, and optionally substituted with 1 or 2 Z³ groups;
R¹ is 6-12 membered aryl, 5-12 membered heteroaryl or 3-12 membered heterocycle, wherein any 6-12 membered aryl, 5-12 membered heteroaryl or 3-12 membered heterocycle of R¹ is optionally substituted with 1, 2, 3, 4, or 5 Z⁴ groups;
R² is phenyl, 5-membered monocyclic-heteroaryl, 6-membered monocyclic-heteroaryl or (C₃-C₇)carbocycle, wherein any phenyl, 5-membered monocyclic-heteroaryl, 6-membered monocyclic-heteroaryl or (C₃-C₇)carbocycle of R² is optionally substituted with 1, 2, 3, 4, or 5 Z⁵ groups;
each R^{3a} and R^{3b} is independently selected from H, halogen, (C₁-C₃)alkyl and (C₁-C₃)haloalkyl, or R^{3a} is selected from H, (C₁-C₃)alkyl and (C₁-C₃)haloalkyl and R^{3b} is selected from —OH and —CN;
Z¹ is selected from 6-12 membered aryl, 5-14 membered heteroaryl and 3-14 membered heterocycle, wherein any 6-12 membered aryl, 5-14 membered heteroaryl and 3-14 membered heterocycle of Z¹ is optionally substituted with 1, 2, 3, 4, or 5 Z^{1a} or Z^{1b};
each Z^{1a} is independently selected from (C₃-C₇)carbocycle, 6-12 membered aryl, 5-12 membered heteroaryl, 3-12 membered heterocycle, halogen, —CN, —ORⁿ¹, —OC(O)R^{p1}, —OC(O)NR^{q1}R^{r1}, —SRⁿ¹, —S(O)R^{p1}, —S(O)₂OH, —S(O)₂R^{p1}, —S(O)₂NR^{q1}R^{r1}, —N R^{q1}R^{r1}, —NRⁿ¹COR^{p1}, —NRⁿ¹CO₂R^{p1}, —NRⁿ¹CONR^{q1}R^{r1}, —NRⁿ¹S(O)₂R^{p1}, —NRⁿ¹S(O)₂R^{p1}, —NRⁿ¹S(O)₂NR^{q1}R^{r1}, NO₂, —C(O)Rⁿ¹, —C(O)ORⁿ¹, —C(O)NR^{q1}R^{r1} and —S(O)₂NRⁿ¹COR^{p1}, wherein any (C₃-C₇)carbocycle, 6-12 membered aryl, 5-12 membered heteroaryl and 3-12 membered heterocycle of Z^{1a} is optionally substituted with 1, 2, 3, 4, or 5 Z^{1c} or Z^{1d} groups;
each Z^{1b} is independently selected from (C₁-C₈)alkyl, (C₂-C₈)alkenyl and (C₂-C₈)alkynyl, wherein any (C₁-C₈)alkyl, (C₂-C₈)alkenyl and (C₂-C₈)alkynyl of Z^{1b} is optionally substituted with 1, 2, 3, 4, or 5 Z^{1c} groups;
each Z^{1c} is independently selected from (C₃-C₇)carbocycle, phenyl, 5-6 membered monocyclic-heteroaryl, 3-7 membered heterocycle, halogen, —CN, —ORⁿ², —OC(O)R^{p2}, —OC(O)NR^{q2}R^{r2}, —SRⁿ², —S(O)R^{p2}, —S(O)₂OH, —S(O)₂R^{p2}, —S(O)₂NR^{q2}R^{r2}, —N R^{q2}R^{r2}, —NRⁿ²COR^{p2}, —NRⁿ²CO₂R^{p2}, —NRⁿ²CONR^{q2}R^{r2}, —NRⁿ²S(O)₂R^{p2}, —NRⁿ²S(O)₂OR^{p2}, —NRⁿ²S(O₂NR^{q2}R^{r2}, NO₂, —C(O)Rⁿ², —C(O)ORⁿ², —C(O)NR^{q2}R^{r2}, halophenyl, 5-6 membered haloheteroaryl, 3-7 membered haloheterocycle and (C₁-C₈)heteroalkyl;
each Z^{1d} is independently selected from (C₁-C₈)alkyl, (C₂-C₈)alkenyl, (C₂-C₈)alkynyl and (C₁-C₈)haloalkyl;
each Rⁿ¹ is independently selected from H, (C₁-C₈)alkyl, (C₂-C₈)alkenyl, (C₂-C₈)alkynyl, (C₃-C₇)carbocycle, 3-7 membered heterocycle, 5-6 membered monocyclic-heteroaryl and phenyl, wherein any (C₃-C₇)carbocycle, 3-7 membered heterocycle, 5-6 membered monocyclic-heteroaryl and phenyl of Rⁿ¹ is optionally substituted with 1, 2, 3, 4, or 5 Z^{1c} or Z^{1d} groups, and wherein any (C₁-C₈)alkyl, (C₂-C₈)alkenyl and (C₂-C₈)alkynyl of Rⁿ¹ is optionally substituted with 1, 2, 3, 4, or 5 Z^{1c} groups;
each R^{p1} is independently selected from (C₁-C₈)alkyl, (C₂-C₈)alkenyl, (C₂-C₈)alkynyl, (C₃-C₇)carbocycle, 3-7 membered heterocycle, 5-6 membered monocyclic-heteroaryl and phenyl, wherein any (C₃-C₇)carbocycle, 3-7 membered heterocycle, 5-6 membered monocyclic-heteroaryl and phenyl of R^{p1} is optionally substituted with 1, 2, 3, 4, or 5 Z^{1c} or Z^{1d} groups, and wherein any (C₁-C₈)alkyl, (C₂-C₈)alkenyl and (C₂-C₈)alkynyl of R^{p1} is optionally substituted with 1, 2, 3, 4, or 5 Z^{1c} groups;
R^{q1} and R^{r1} are each independently selected from H, (C₁-C₈)alkyl, (C₂-C₈)alkenyl, (C₂-C₈)alkynyl, (C₃-C₇)carbocycle, 3-7 membered heterocycle, 5-6 membered monocyclic-heteroaryl and phenyl, wherein any (C₃-C₇)carbocycle, 3-7 membered heterocycle, 5-6 membered monocyclic-heteroaryl and phenyl of R^{q1} or R^{r1} is optionally substituted with 1, 2, 3, 4, or 5 Z^{1c} or Z^{1d} groups, and wherein any (C₁-C₈)alkyl, (C₂-C₈)alkenyl and (C₂-C₈)alkynyl of R^{q1} or R^{r1} is optionally substituted with 1, 2, 3, 4, or 5 Z^{1c} groups, or R^{q1} and R^{r1} together with the nitrogen to which they are attached form a 5, 6 or 7-membered heterocycle, wherein the 5, 6 or 7-membered heterocycle is optionally substituted with 1, 2, 3, 4, or 5 Z^{1c} or Z^{1d} groups;
each Rⁿ² is independently selected from H, (C₁-C₈)alkyl, (C₂-C₈)alkenyl, (C₂-C₈)alkynyl, (C₃-C₇)carbocycle, 3-7 membered heterocycle, 5-6 membered monocyclic-heteroaryl, phenyl, halophenyl, 5-6 membered monocyclic-haloheteroaryl, 3-7 membered haloheterocycle, (C₁-C₈)haloalkyl and (C₁-C₈)heteroalkyl;
each R^{p2} is independently selected from (C₁-C₈)alkyl, (C₂-C₈)alkenyl, (C₂-C₈)alkynyl, (C₃-C₇)carbocycle, 3-7 membered heterocycle, 5-6 membered monocyclic-heteroaryl, phenyl, halophenyl, 5-6 membered monocyclic-haloheteroaryl, 3-7 membered haloheterocycle, (C₁-C₈)haloalkyl and (C₁-C₈)heteroalkyl;
R^{q2} and R^{r2} are each independently selected from H, (C₁-C₈)alkyl, (C₂-C₈)alkenyl, (C₂-C₈)alkynyl, (C₃-C₇)carbocycle, 3-7 membered heterocycle, 5-6 membered monocyclic-heteroaryl, phenyl, halophenyl, 5-6 membered monocyclic-haloheteroaryl, 3-7 membered haloheterocycle, (C₁-C₈)haloalkyl and (C₁-C₈)heteroalkyl, or R^{q2} and R^{r2} together with the nitrogen to which they are attached form a 5, 6 or 7-membered heterocycle;
Z² is selected from (C₂-C₈)alkenyl, (C₂-C₈)alkynyl, 6-12 membered aryl, 5-12 membered C-linked-heteroaryl, 3-12 membered C-linked-heterocycle, —C(O)Rⁿ³ and —C(O)NR^{q3}R^{r3}, wherein any 6-12 membered aryl, 5-12 membered C-linked-heteroaryl and 3-12 membered C-linked-heterocycle of Z² is optionally substituted with 1, 2, 3, 4, or 5 Z^{2b} or Z^{2c} groups, and wherein any (C₂-C₈)alkenyl and (C₂-C₈)alkynyl of Z² is optionally substituted with 1, 2, 3, 4, or 5 Z^{2c} groups;
each Z^{2a} is independently selected from (C₃-C₇)carbocycle, 6-12 membered aryl, 5-12 membered heteroaryl, 3-12 membered heterocycle, halogen, —CN, —ORⁿ⁴, —OC(O)R^{p4}, —OC(O)NR^{q4}R^{r4}, —SRⁿ⁴, —S(O)R^{p4}, —S(O)₂OH, —S(O)₂R^{p4}, —S(O)₂NR^{q4}R^{r4}, —N R^{q4}R^{r4}, —NRⁿ⁴COR^{p4}, —NRⁿ⁴CO₂R^{p4}, —NRⁿ⁴CONR^{q4}R^{r4}, —NRⁿ⁴S(O)₂R^{p4}, —NRⁿ⁴S(O)₂OR^{p4}, —NRⁿ⁴S(O)₂NR^{q4}R^{r4}, NO₂, —C(O)Rⁿ⁴, —C(O)ORⁿ⁴ and —C(O)NR^{q4}R^{r4}, wherein any (C₃-C₇)carbocycle, 6-12 membered aryl, 5-12 membered heteroaryl and 3-12 membered heterocycle of Z^{2a} is optionally substituted with 1, 2, 3, 4, or 5 Z^{2b} or Z^{2c} groups;
each Z^{2b} is independently selected from (C₁-C₄)alkyl, (C₁-C₄)heteroalkyl and (C₁-C₄)haloalkyl;
each Z^{2c} is independently selected from halogen, —CN, —ORⁿ⁴, —OC(O)R^{p4}, —OC(O)NR^{q4}R^{r4}, —SRⁿ⁴, —S(O)R^{p4}, —S(O)₂OH, —S(O)₂R^{p4}, —S(O)₂NR^{q4}R^{r4}, —N R^{q4}R^{r4}, —NRⁿ⁴COR^{p4}, —NRⁿ⁴CO₂R^{p4}, —NRⁿ⁴CONR^{q4}R^{r4}, —NRⁿ⁴S(O)₂R^{p4}, —NRⁿ⁴S(O)₂OR^{p4}, —NRⁿ⁴S(O)₂NR^{q4}R^{r4}, NO₂, —C(O)Rⁿ⁴, —C(O)ORⁿ⁴ and —C(O)NR^{q4}R^{r4};
each Rⁿ³ is independently selected from H, (C₁-C₄)alkyl, (C₂-C₄)alkenyl, (C₃-C₇)carbocycle, 3-12 membered heterocycle, 5-12 membered heteroaryl and 6-12 membered aryl, wherein any (C₃-C₇)carbocycle, 3-12 membered heterocycle, 5-12 membered heteroaryl and 6-12 membered aryl of Rⁿ³ is optionally substituted with 1, 2, 3, 4, or 5 Z^{2b} or Z^{2c} groups, and wherein any (C₁-C₄)alkyl, (C₂-C₄)alkenyl and (C₂-C₄)alkynyl of Rⁿ³ is optionally substituted with 1, 2, 3, 4, or 5 Z^{2a} groups;
R^{q3} and R^{r3} are each independently selected from H, (C₁-C₄)alkyl, (C₂-C₄)alkenyl, (C₃-C₇)carbocycle, 3-12 membered heterocycle, 5-12 membered heteroaryl and 6-12 membered aryl, wherein any (C₃-C₇)carbocycle, 3-12 membered heterocycle, 5-12 membered heteroaryl and 6-12 membered aryl of R^{q3} or R^{r3} is optionally substituted with one or more (e.g., 1, 2, 3, 4 or 5) Z^{2b} or Z^{2c} groups, and wherein any (C₁-C₄)alkyl and (C₂-C₄)alkenyl of R^{q3} or R^{r3} is optionally substituted with 1, 2, 3, 4, or 5 Z^{2a} groups, or R^{q3} and R^{r3} together with the nitrogen to which they are attached form a heterocycle or heteroaryl, wherein the heterocycle or heteroaryl is optionally substituted with 1, 2, 3, 4, or 5 Z^{2b} or Z^{2c} groups;
each Rⁿ⁴ is independently selected from H, (C₁-C₄)alkyl, (C₂-C₈)alkenyl, (C₂-C₈)alkynyl, (C₁-C₄)haloalkyl and (C₁-C₄)heteroalkyl;
each R^{p4} is independently selected from (C₁-C₈)alkyl, (C₂-C₄)alkenyl, (C₂-C₄)alkynyl, (C₁-C₄)haloalkyl and (C₁-C₄)heteroalkyl;
R^{q4} and R^{r4} are each independently selected from H, (C₁-C₄)alkyl, (C₂-C₄)alkenyl, (C₂-C₄)alkynyl, (C₁-C₄)haloalkyl and (C₁-C₄)heteroalkyl;
each Z³ is independently selected from halogen, (C₁-C₄)alkyl, —OH, —CN, (C₁-C₄)heteroalkyl and (C₁-C₄)haloalkyl;
each Z⁴ is independently selected from (C₁-C₈)alkyl, (C₂-C₈)alkenyl, (C₂-C₈)alkynyl, (C₃-C₇)carbocycle, halogen, —CN, —ORⁿ⁵, —OC(O)R^{p5}, —OC(O)NR^{q5}R^{r5}, —SRⁿ⁵, —S(O)R^{p5}, —S(O)₂OH, —S(O)₂R^{p5}, —S(O)₂NR^{q5}R^{r5}, —N R^{q5}R^{r5}, —NRⁿ⁵COR^{p5}, —NRⁿ⁵CO₂R^{p5}, —NRⁿ⁵CONR^{q5}R^{r5}, —NRⁿ⁵S(O)₂R^{p5}, —NRⁿ⁵S(O)₂OR^{p5}, —NRⁿ⁵S(O)₂NR^{q5}R^{r5}, NO₂, —C(O)Rⁿ⁵, —C(O)ORⁿ⁵ and —C(O)NR^{q5}R^{r5}, wherein any (C₃-C₇)carbocycle, of Z⁴ is optionally substituted with 1, 2, 3, 4, or 5 Z^{4a} or Z^{4b} groups, and wherein any (C₁-C₈)alkyl, (C₂-C₈)alkenyl and (C₂-C₈)alkynyl of Z⁴ is optionally substituted with 1, 2, 3, 4, or 5 Z^{4a} groups;
each Z^{4a} is independently selected from halogen, —CN, —ORⁿ⁶, —OC(O)R^{p6}, —OC(O)NR^{q6}R^{r6}, —SRⁿ⁶, —S(O)R^{p6}, —S(O)₂OH, —S(O)₂R^{p6}, —S(O)₂NR^{q6}R^{r6}, —N R^{q6}R^{r6}, —NRⁿ⁶COR^{p6}, —NRⁿ⁶CO₂R^{p6}, —NRⁿ⁶CONR^{q6}R^{r6}, —NRⁿ⁶S(O)₂R^{p6}, —NRⁿ⁶S(O)₂OR^{p6}, —NRⁿ⁶S(O)₂NR^{q6}R^{r6}, NO₂, —C(O)Rⁿ⁶, —C(O)ORⁿ⁶ and —C(O)NR^{q6}R^{r6};
each Z^{4b} is independently selected from (C₁-C₄)alkyl, (C₂-C₄)alkenyl (C₂-C₄)alkynyl and (C₁-C₄)haloalkyl;
each Rⁿ⁵ is independently selected from H, (C₁-C₄)alkyl, (C₁-C₄)haloalkyl, (C₁-C₄)heteroalkyl, (C₂-C₄)alkenyl and (C₂-C₄)alkynyl;
each R^{p5} is independently selected from (C₁-C₄)alkyl, (C₁-C₄)haloalkyl, (C₁-C₄)heteroalkyl, (C₂-C₄)alkenyl and (C₂-C₄)alkynyl;
R^{q5} and R^{r5} are each independently selected from H, (C₁-C₄)alkyl, (C₁-C₄)haloalkyl, (C₁-C₄)heteroalkyl, (C₂-C₄)alkenyl and (C₂-C₄)alkynyl;
each Rⁿ⁶ is independently selected from H, (C₁-C₄)alkyl, (C₁-C₄)haloalkyl, (C₁-C₄)heteroalkyl, (C₂-C₄)alkenyl and (C₂-C₄)alkynyl;
each R^{p6} is independently selected from (C₁-C₄)alkyl, (C₁-C₄)haloalkyl, (C₁-C₄)heteroalkyl, (C₂-C₄)alkenyl and (C₂-C₄)alkynyl;
R^{q6} and R^{r6} are each independently selected from H, (C₁-C₄)alkyl, (C₁-C₄)haloalkyl, (C₁-C₄)heteroalkyl, (C₂-C₄)alkenyl and (C₂-C₄)alkynyl;
each Z⁵ is independently selected from (C₁-C₆)alkyl, halogen, —CN and —ORⁿ⁷, wherein any (C₁-C₆)alkyl of Z⁵ is optionally substituted with 1, 2, 3, 4, or 5 halogen; and
each Rⁿ⁷ is independently selected from H, (C₁-C₃)alkyl, (C₁-C₃)haloalkyl and (C₃-C₇)carbocycle;
or a pharmaceutically acceptable salt thereof.

2. The compound of claim 1 which is a compound of formula Ia: 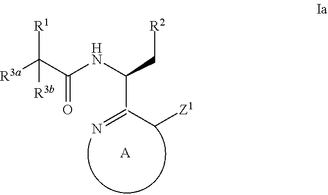
or a pharmaceutically acceptable salt thereof.

3. The compound of claim 1, or a pharmaceutically acceptable salt thereof, wherein R^{3a} and R^{3b} are each H.

4. (canceled)

5. The compound of claim 1, or a pharmaceutically acceptable salt thereof, wherein R² is phenyl optionally substituted with 1, 2, 3, 4, or 5 Z⁵ groups.

6-7. (canceled)

8. The compound of claim 1, or a pharmaceutically acceptable salt thereof, wherein R² is 3,5-difluorophenyl.

9. (canceled)

10. The compound of claim 1 which is a compound of formula Ie: 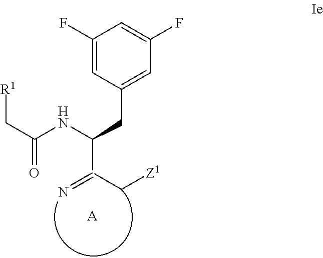
or a pharmaceutically acceptable salt thereof.

11-13. (canceled)

14. The compound of claim 1, or a pharmaceutically acceptable salt thereof, wherein A-Z¹ is selected from: 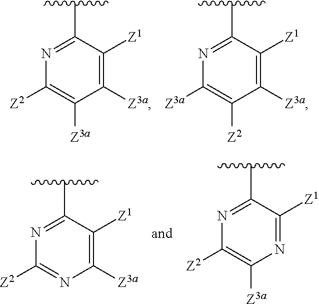
wherein each Z^{3a} is independently selected from H and Z³.

15-21. (canceled)

22. The compound of claim 1, or a pharmaceutically acceptable salt thereof, wherein Z¹ is selected from phenyl, 5-6 membered monocyclic-heteroaryl, 8-10 membered bicyclic-heteroaryl, 8-10 membered bicyclic-heterocycle and 9-12 membered tricyclic-heterocycle, wherein the 5-6 membered monocyclic-heteroaryl, 8-10 membered bicyclic-heteroaryl, 8-10 membered bicyclic-heterocycle and 9-12 membered tricyclic-heterocycle have 1-11 carbon atoms and 1-5 heteroatoms in the ring system, and wherein any phenyl, 5-6 membered monocyclic-heteroaryl, 8-10 membered bicyclic-heteroaryl, 8-10 membered bicyclic-heterocycle and 9-12 membered tricyclic-heterocycle of Z¹ is optionally substituted with 1, 2, 3, 4, or 5 Z^{1a} or Z^{1b} groups.

23-29. (canceled)

30. The compound of claim 1, or a pharmaceutically acceptable salt thereof, wherein Z¹ is selected from:

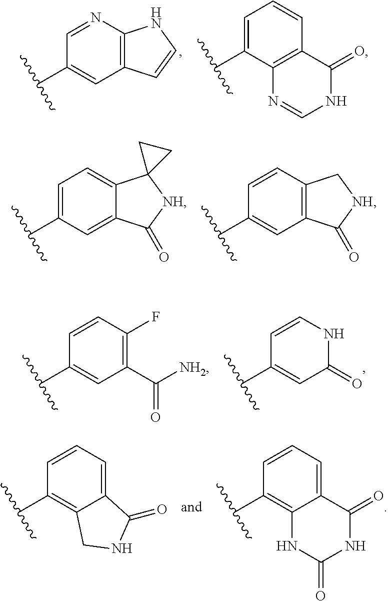

31-32. (canceled)

33. The compound of claim 1, or a pharmaceutically acceptable salt thereof, wherein Z² is selected from (C₂-C₈)alkynyl, phenyl, 5-6 membered C-linked-monocyclic-heteroaryl, 8-10 membered C-linked-bicyclic-heteroaryl, 8-10 membered C-linked-bicyclic-heterocycle and —C(O)NR^{q3}R^{r3}, wherein the 5-6 membered C-linked-monocyclic-heteroaryl, 8-10 membered C-linked-bicyclic-heteroaryl and 8-10 membered C-linked-bicyclic-heterocycle have 1-9 carbon atoms and 1-4 heteroatoms in the ring system, and wherein any phenyl, 5-6 membered C-linked-monocyclic-heteroaryl, 8-10 membered C-linked-bicyclic-heteroaryl, 8-10 membered and C-linked-bicyclic-heterocycle of Z² is optionally substituted with 1, 2, 3, 4, or 5 Z^{2b} or Z^{2c} groups, and wherein any (C₂-C₈)alkynyl of Z² is optionally substituted with 1, 2, 3, 4, or 5 Z^{2c} groups.

34-37. (canceled)

38. The compound of claim 1, or a pharmaceutically acceptable salt thereof, wherein each Z^{2c} is independently selected from halogen, —ORⁿ⁴ and —C(O)NR^{q4}R^{r4}.

39. (canceled)

40. The compound of claim 1, or a pharmaceutically acceptable salt thereof, wherein Z² is selected from:

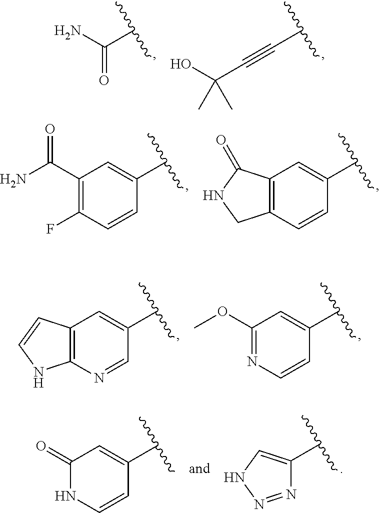

41. The compound of claim 1, or a pharmaceutically acceptable salt thereof, wherein A-Z¹ is selected from:

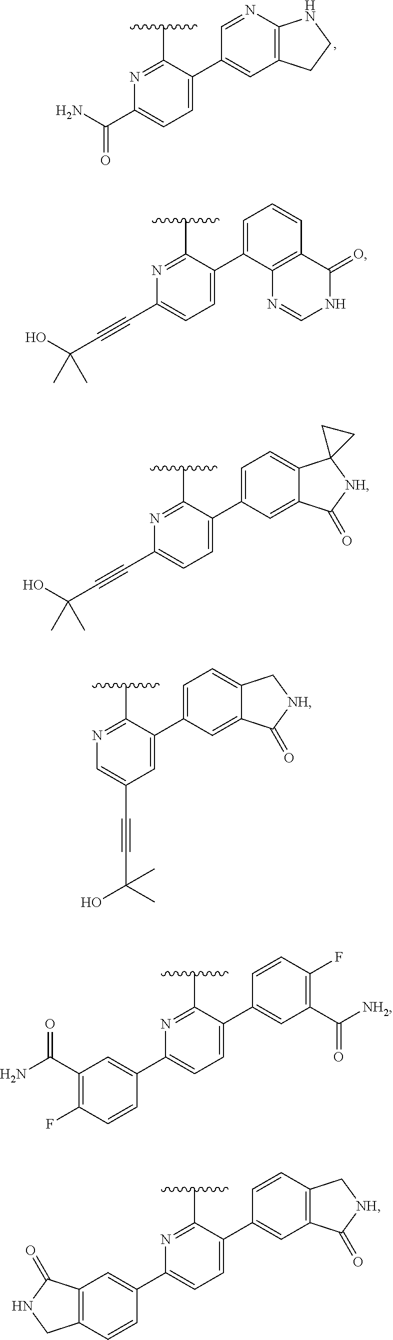 [SMILES: *c1nc(C(N)=O)ccc1-c1cnc2c(c1)CCN2 ; *c1nc(C#CC(C)(C)O)ccc1-c1cccc2c(=O)[nH]cnc12 ; *c1nc(C#CC(C)(C)O)ccc1-c1ccc2c(c1)C(=O)NC21CC1 ; *c1ncc(C#CC(C)(C)O)cc1-c1ccc2c(c1)C(=O)NC2 ; *c1nc(-c2ccc(F)c(C(N)=O)c2)ccc1-c1ccc(F)c(C(N)=O)c1 ; *c1nc(-c2ccc3c(c2)C(=O)NC3)ccc1-c1ccc2c(c1)C(=O)NC2]

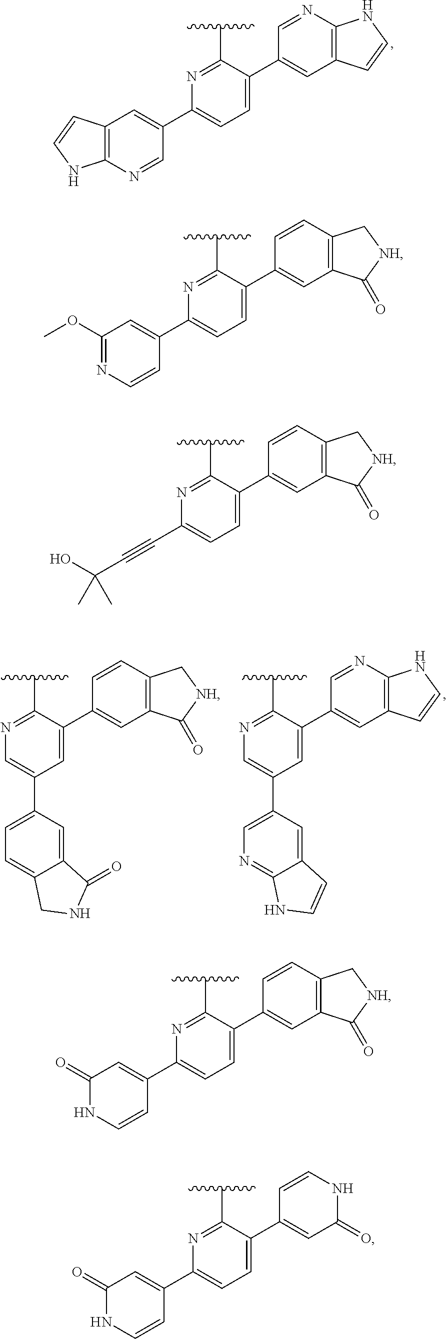

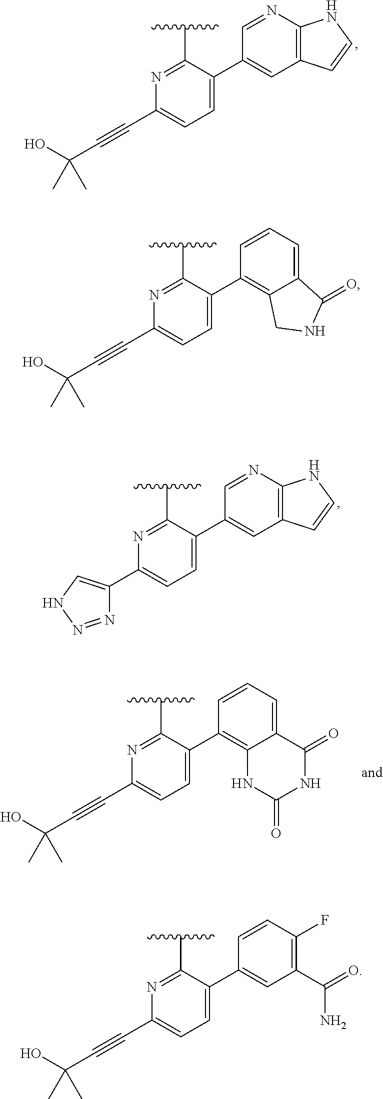 [SMILES: *c1nc(C#CC(C)(C)O)ccc1-c1cnc2[nH]ccc2c1 ; *c1nc(C#CC(C)(C)O)ccc1-c1cccc2c1CNC2=O ; *c1nc(-c2c[nH]nn2)ccc1-c1cnc2[nH]ccc2c1 ; *c1nc(C#CC(C)(C)O)ccc1-c1cccc2c(=O)[nH]c(=O)[nH]c12 ; *c1nc(C#CC(C)(C)O)ccc1-c1ccc(F)c(C(N)=O)c1]

42-43. (canceled)

44. The compound of claim 1, or a pharmaceutically acceptable salt thereof, wherein R¹ is a 8-12 membered bicyclic-heteroaryl or 8-12 membered tricyclic-heteroaryl, wherein the 8-12 membered bicyclic-heteroaryl or 8-12 membered tricyclic-heteroaryl have 4-10 carbon atoms and 1-5 heteroatoms in the ring system, and wherein any 8-12 membered bicyclic-heteroaryl or 8-12 membered tricyclic-heteroaryl of R¹ is optionally substituted with 1, 2, 3, 4, or 5 Z⁴ groups.

45-46. (canceled)

47. The compound of claim 1, or a pharmaceutically acceptable salt thereof, wherein R¹ has the following formula IId: 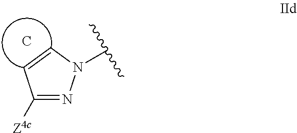
wherein:
C together with the two carbon atoms to which it is attached forms a 3-7 membered monocyclic-carbocycle, 5-9 membered bicyclic-carbocycle, 3-7 membered monocyclic-heterocycle or 5-9 membered bicyclic heterocycle, wherein any 3-7 membered monocyclic-carbocycle, 5-9 membered bicyclic-carbocycle, 3-7 membered monocyclic-heterocycle or 5-9 membered bicyclic heterocycle of C is optionally substituted with one 1, 2, 3, 4, or 5 Z⁴ groups; and
each Z^{4c} is independently selected from H or Z⁴.

48-50. (canceled)

51. The compound of claim 1, or a pharmaceutically acceptable salt thereof, wherein each Z⁴ is independently selected from fluoro, trifluoromethyl and difluoromethyl.

52-54. (canceled)

55. A pharmaceutical composition comprising a compound of claim 1, or a pharmaceutically acceptable salt thereof, and a pharmaceutically acceptable carrier.

56. A method for treating an HIV virus infection in a human comprising administering a therapeutically effective amount of a compound of claim 1, or a pharmaceutically acceptable salt thereof, to the human.

57. (canceled)

58. A method for treating an HIV infection in a human comprising administering to the human in need thereof a therapeutically effective amount of a compound of claim 1, or a pharmaceutically acceptable salt thereof, in combination with a therapeutically effective amount of one or more additional therapeutic agents selected from the group consisting of HIV protease inhibiting compounds, HIV non-nucleoside inhibitors of reverse transcriptase, HIV nucleoside inhibitors of reverse transcriptase, HIV nucleotide inhibitors of reverse transcriptase, HIV integrase inhibitors, gp41 inhibitors, CXCR4 inhibitors, gp120 inhibitors, CCR5 inhibitors, capsid polymerization inhibitors, and other drugs for treating HIV, and combinations thereof.

59-62. (canceled)
